(19) United States
(12) Patent Application Publication
(10) Pub. No.: US 2021/0322577 A1
(43) Pub. Date: Oct. 21, 2021

(54) METHODS AND SYSTEMS FOR MODIFYING DNA

(71) Applicant: FLAGSHIP PIONEERING INNOVATIONS V, INC., Cambridge, MA (US)

(72) Inventors: Laura Gabriela Lande, Chestnut Hill, MA (US); David Arthur Berry, Newton, MA (US); Andrew Bogorad, Brookline, MA (US)

(21) Appl. No.: 16/490,331

(22) PCT Filed: Mar. 2, 2018
(86) PCT No.: PCT/US18/20558

§ 371 Date: Aug. 30, 2019

Related U.S. Application Data
(60) Provisional application No. 62/466,698, filed on Mar. 3, 2017.

(51) Int. Cl. A61K 48/00; C12N 15/11; C12N 9/10; A61K 38/45; A61K 47/64; A61K 47/54

(52) U.S. Cl. CPC A61K 48/0066; C12N 15/11; C12N 9/1007; A61K 47/549; A61K 38/45; A61K 47/64; C12Y 201/01037

(57) ABSTRACT

The present disclosure provides technologies for modulating gene expression.

### CROSS-REFERENCE TO RELATED APPLICATIONS

The present application claims priority to and benefit from U.S. provisional application No. 62/466,698, filed on Mar. 3, 2017, the contents of which are herein incorporated by reference.

### BACKGROUND

Many diseases are caused by defective regulation of expression of certain genes.

### SUMMARY

The aspects as described here may be utilized with any one or more of the embodiments delineated herein. The present disclosure provides technologies (e.g. compositions, methods, systems, etc.) capable of modulating certain genes.

In some aspects, the present disclosure provides systems comprising a first composition comprising: a first component comprising a first DNA targeting moiety capable of binding to a first target DNA site, operably linked to a first incomplete effector moiety (e.g., having less than 90%, less than 80%, less than 70%, less than 60%, less than 50%, less than 40%, less than 30%, less than 20%, less than 10%, less than 5%, or less than 1% of the effector activity of the effector protein); and a second composition comprising: a second component comprising a second DNA targeting moiety capable of binding to a second target DNA site adjacent to the first target site, operably linked to a second incomplete effector moiety (e.g., having less than 90%, less than 80%, less than 70%, less than 60%, less than 50%, less than 40%, less than 30%, less than 20%, less than 10%, less than 5%, or less than 1% of the effector activity of the effector protein), wherein the first and second component are capable of interacting to provide an effector activity (e.g., restoring at least 50%, 60%, 70%, 80%, 85%, 90%, 95% or more effector activity) at or near the target site.

In some embodiments, the effector activity modulates the DNA at or near the target site. In some embodiments, the effector activity is selected from the group consisting of DNA methyltransferase, histone methyltransferase, deaminase, acetyltransferase, histone deacetylase, ligase, nuclease, phosphatase, recombinase, transposase, and polynucleotide kinase activity.

In some embodiments, the first and second composition are operably linked. In some embodiments, at least one composition of the system further comprises a nanoparticle, liposome, or exosome. In some embodiments, at least one composition of the system further comprises a membrane penetrating polypeptide. In some embodiments, the first and second compositions are each formulated as a separate pharmaceutical composition. In some embodiments, the first and second compositions are formulated in a single pharmaceutical composition.

In some embodiments, the first and second components bind a DNA sequence comprising the first and second target sites. In one embodiment, the DNA sequence comprises a transcriptional control sequence. In one embodiment, the DNA sequence is genomic DNA. In some embodiments, the first and second component prevents, inhibits, and/or interferes with an activity of an endogenous effector protein at the target site.

In some embodiments, the incomplete effector moieties are derived from at least one effector selected from the group consisting DNA methyltransferases (e.g., DNMT3a, DNMT3b, DNMTL, DRM2), DNA demethylation (e.g., the TET family, DME), histone methyltransferase, deaminase, acetyltransferase, histone deacetylase (e.g., HDAC1, HDAC2, HDAC3), sirtuin 1, 2, 3, 4, 5, 6, or 7, lysine-specific histone demethylase 1 (LSD1), histone-lysine-N-methyltransferase (Setdb1), euchromatic histone-lysine N-methyltransferase 2 (G9a), histone-lysine N-methyltransferase (SUV39H1), enhancer of zeste homolog 2 (EZH2), viral lysine methyltransferase (vSET), histone methyltransferase (SET2), protein-lysine N-methyltransferase (SMYD2), ligases, nucleases (e.g., endonucleases, T7, Cpf1, Cas9, zinc finger nuclease), phosphatases (e.g., alkaline phosphatases), recombinases (e.g., Cre), transposases (Tn3, Tn5, Sleeping Beauty), polynucleotide kinases (e.g., T4), enzymes with a role in DNA repair (e.g., RecA, N-glycosylase, AP-lyase), enzymes with a role in DNA demethylation (e.g., the TET family enzymes catalyze oxidation of 5-methylcytosine to 5-hydroxymethylcytosine and higher oxidative derivatives), and fragments or variants thereof. In some embodiments, the incomplete effector moieties are derived from at least one effector selected from the group consisting of Table 1 from Park et al, Genome Biology, 2016, 17:183. In some embodiments, the incomplete effector moieties are described in Example 1, Example 4, Example 6, Example 8, or Example 10.

In some embodiments, the DNA targeting moieties are described in Example 2, Example 3, Example 5, Example 7, or Example 9. In some embodiments, at least one of the DNA targeting moieties are RNA.

In some aspects, the present disclosure provides a system comprising: a) a first nucleic acid sequence encoding a first incomplete effector moiety (e.g., having less than 90%, less than 80%, less than 70%, less than 60%, less than 50%, less than 40%, less than 30%, less than 20%, less than 10%, less than 5%, or less than 1% of the effector activity of the effector protein); b) a first DNA targeting moiety that interacts with the first incomplete effector moiety and binds to a first target DNA site; c) a second nucleic acid sequence encoding a second incomplete effector moiety (e.g., having less than 90%, less than 80%, less than 70%, less than 60%, less than 50%, less than 40%, less than 30%, less than 20%, less than 10%, less than 5%, or less than 1% of the effector activity of the effector protein); and d) a second DNA targeting moiety that interacts with the second incomplete effector moiety and binds to a second target DNA site adjacent to the first target site, wherein the first and second incomplete effector moieties interact to provide an effector activity (e.g., restoring at least 50%, 60%, 70%, 80%, 85%, 90%, 95% or more effector activity) at or near the target site.

In some embodiments, the system further comprises one or more vectors comprising one or more of a) through d). In one embodiment, the vector is an expression vector.

In some embodiments, a) and b) are operably linked and c) and d) are operably linked. In some embodiments, a) comprises a first functional group and the first incomplete effector moiety comprises a first complementary functional group; and b) comprises a second functional group and the second incomplete effector moiety comprises a second complementary functional group, wherein the first functional group interacts with the first complementary functional group and the second functional group interacts with the second complementary functional group.

In some embodiments, the system is formulated as a pharmaceutical composition.

In some aspects, the present disclosure includes a pharmaceutical composition comprising a cell modified to express the system described herein.

In some aspects, the present disclosure provides a method of modifying a target site, the method comprising: binding a first component comprising a first incomplete effector moiety (e.g., having less than 90%, less than 80%, less than 70%, less than 60%, less than 50%, less than 40%, less than 30%, less than 20%, less than 10%, less than 5%, or less than 1% of the effector activity of the effector protein) with a nucleic acid sequence adjacent to the target site; and binding a second component comprising a second incomplete effector moiety (e.g., having less than 90%, less than 80%, less than 70%, less than 60%, less than 50%, less than 40%, less than 30%, less than 20%, less than 10%, less than 5%, or less than 1% of the effector activity of the effector protein) with a different nucleic acid sequence also adjacent to the target site, wherein binding both components to the nucleic acid sequences allows interaction between the first and second components to induce effector activity (e.g., restoring at least 50%, 60%, 70%, 80%, 85%, 90%, 95% or more effector activity) at the target site.

In some embodiments, the effector activity is selected from the group consisting of DNA methyltransferase, histone methyltransferase, deaminase, acetyltransferase, histone deacetylase, ligase, nuclease, phosphatase, recombinase, transposase, and polynucleotide kinase activity. In some embodiments, the effector activity at the target site modulates gene expression. In some embodiments, binding both components modulates chromatin topology and/or chromatin structure. In some embodiments, binding both components prevents, inhibits, and/or interferes with an activity of an endogenous effector protein at the target site.

In some aspects, the present disclosure provides a method of treating a disease or condition comprising administering the system described herein to a subject in need thereof.

In some embodiments, the system comprises a methyltransferase to treat (e.g., sufficient to decrease or inhibit expression by at least 5%, 10%, 15%, 20%, 25%, 30%, 35%, 40%, 45%, 50%, 55%, 60%, 65% 70%, 75%, 80%, 85%, 90%, 95% or greater) a disease characterized by an overexpressed/dominant negative gene such as: an oncogene driven cancer (e.g MYC addicted cancers, Bcr-Abl), severe congenital neutropenia, and Huntington's chorea. In some embodiments, the system comprises a demethylase to treat a disease characterized by under-expression of a gene: an imprinted disease (e.g. Prader Willi, Angelman Syndrome), a haploinsufficient disease (e.g Dravet's syndrome, Familial hypertriglyceridemia), Fragile X, Rett Syndrome, and a tumor suppressor that is underactive (e.g., retinoblastoma). In some embodiments, the system is effective in treating and/or reducing symptoms of or associated with one or more of the diseases, disorders and/or conditions as described herein.

### Definitions

The term “adjacent”, as used herein in reference to a sequence, refers to a sequence near or in proximity, e.g., structural proximity, e.g., two or three-dimensional proximity, to another sequence. The sequences adjacent to one another may be contiguous or non-contiguous. Two sites may be adjacent to each other if they are separated by the distance spanned by the association of two incomplete effectors when they come together to make an active effector.

The term “derived from” as used herein, refers to a source, e.g., an original compound or sequence. A compound or sequence may be derived from a larger source compound or sequence, or a variant of a source compound or sequence.

The term “DNA targeting moiety” as used herein, refers to a molecule that specifically binds a sequence in or around a gene. Examples of a DNA targeting moiety include, but are not limited to, an oligonucleotide, e.g., DNA, RNA, e.g., a guide RNA, a nucleic acid encoding a guide RNA, a PNA, a peptide beta, a peptide gamma, a DNA binding protein (e.g., a TALE, a Zn finger, a bHLH domain protein; a leucine zipper, or functional fragment or variant thereof).

The term “effector” as used herein means a molecule with biological activity, e.g., DNA or histone modulating activity. In embodiments, an effector is a protein such as an enzyme that modulates DNA or chromatin (e.g., histones).

As used herein, the term “fragment” refers to a nucleic acid or amino acid sequence comprising a portion (e.g., 5%, 10%, 15%, 20%, 25%, 30%, 35%, 40%, 45%, 50%, 55%, 60%, 65%, 70%, 75%, 80%, 85%, 90%, 95% or any portion thereof) of the contiguous residues of a nucleotide or amino acid sequence of interest.

The term “incomplete effector moiety” as used herein, refers to a molecule that does not display 100% of the activity of a reference effector. When physically proximate, two or more incomplete effector moieties interact to provide an effector activity, e.g., to reconstitute substantially complete effector activity.

The term “operably linked” refers to functional relationship between two molecules, e.g., between a sequence (e.g., polynucleotide or polypeptide) and another sequence (e.g., polynucleotide or polypeptide). For example, a nucleic acid sequence is operably linked with a polypeptide sequence when the nucleic acid sequence is placed in a functional linkage with the polypeptide sequence. For instance, a first moiety is operably linked to second moiety if the first moiety is positioned to enable a function of the second moiety; a promoter or enhancer is operably linked to a coding sequence if it affects the transcription of the sequence; or a nucleic acid sequence is operably linked to a coding sequence if it is positioned so as to facilitate translation.

The term “target site” as used herein, refers to a nucleic acid sequence of interest that may be modulated (e.g., methylation or demethylation) to increase or decrease transcription of a gene.

“Treatment” and “treating,” as used herein, refer to the medical management of a subject with the intent to improve, ameliorate, stabilize, prevent or cure a disease, pathological condition, or disorder. This term includes active treatment (treatment directed to improve the disease, pathological condition, or disorder), causal treatment (treatment directed to the cause of the associated disease, pathological condition, or disorder), palliative treatment (treatment designed for the relief of symptoms), preventative treatment (treatment directed to minimizing or partially or completely inhibiting the development of the associated disease, pathological condition, or disorder); and supportive treatment (treatment employed to supplement another therapy).

As used herein, the term “variant” refers to one or more amino acid substitutions, additions, or deletions (for example, 1, 2, 3, 4, 5, 6, 7, 8, 9, 10 or more, optionally 11-20, 21-30 or more, for example up to 10% of a polypeptide or nucleic acid), wherein the variant still maintains one or more functions (e.g. completely, partially, minimally) of the starting polypeptide. For example, non-limiting examples of conservative amino acid substitutions

### BRIEF DESCRIPTION OF THE DRAWING

The following detailed description of the embodiments of the present disclosure will be better understood when read in conjunction with the appended drawings. For the purpose of illustrating the present disclosure, there are shown in the drawings embodiments, which are presently exemplified. It should be understood, however, that the present disclosure is not limited to the precise arrangement and instrumentalities of the embodiments shown in the drawings.

FIG. 1 shows the sequence of the PCSK9 promoter. The target CpG-rich area is underlined. CpG sites are highlighted in green. Targets of guiding ssDNA strands are highlighted in teal. 5′UTR is highlighted in grey.

FIG. 2 shows a ClustalO alignment of prokaryotic HhaI and HSssI with human DNMT3A. The N- and C-terminal fragments of HSssI and DNMT3A are highlighted in green and yellow, respectively, with the overlapping sequence highlighted in teal.

FIG. 3 is a stereo view of the HSssI homolog, HhaI, complexed with DNA. HhaI residues corresponding to the HSssI N- and C-terminal fragments are colored green and yellow, respectively. The overlap is colored teal. Sequence alignment performed using ClustalO (see FIG. 2). Coloring is maintained from FIG. 2.

FIG. 4 is a stereo view of the catalytic domain of eukaryotic DNMT3A. The N-terminal and C-terminal fragments are highlighted in green and yellow, respectively.

FIG. 5 shows the sequence of the ELANE promoter. The target CpG-rich area is underlined. CpG sites are highlighted in green. Targets of guiding ssDNA strands are highlighted in teal. 5′UTR is highlighted in grey.

FIG. 6 shows the sequence of the FMR1 promoter. The target CpG-rich area is underlined. CpG sites are highlighted in green. Targets of guiding ssDNA strands are highlighted in teal. 5′UTR is highlighted in grey.

FIG. 7 is a stereo view of Cpf1 complexed with cRNA and target DNA. The N-terminal and C-terminal fragments are colored green and yellow, respectively.

FIG. 8 shows the sequence of the BCR promoter. The four MYC binding sites are underlined. The MYC binding site chosen for deletion is highlighted in purple. Targets of guiding ssDNA strands are highlighted in teal. 5′UTR is highlighted in grey.

FIG. 9 is an illustration showing an overlay of N. tabacum DRM1 and eukaryotic DNMT3A structures. DNMT3A is colored grey. The catalytic and TRD domains of DRM1 are colored purple and blue.

FIG. 10 shows a ClustalO alignment of the related enzymes N. tabacum DRM1 and A. thaliana DRM2. The N- and C-terminal fragments of DRM2 are highlighted in green and yellow, respectively.

FIG. 11 shows a stereo view of the DRM2 homolog, DRM1. DRM2 residues corresponding to the N- and C-terminal fragments described above are colored green and yellow, respectively. Sequence alignment performed using ClustalO (see FIG. 10 for color scheme).

FIG. 12 shows the FWA promoter. A pair of tandem repeats found within the FWA promoter is underlined. The CpG sites within the tandem repeat are highlighted in green. The start codon is highlighted in purple. Targets of guiding ssDNA strands are highlighted in teal.

FIG. 13 is an illustration showing essential elements for DME catalytic activity. Top: The three required domains are shown in the context of wild-type DME (Domain A, the glycosylase domain, and Domain B), as well as the poorly conserved interdomain regions (IDR1 connecting Domain A and the glycosylase domain, and IDR2 connecting the glycosylase domain and Domain B). Bottom: Minimum construct that retains catalytic activity, with sequence of the artificial linker replacing IDR1 shown.

FIG. 14 shows the amino acid sequence of A. thaliana DME. The N-terminal fragment described above is highlighted in green and contains Domain A. The C-terminal fragment described above is highlighted in yellow and contains the glycosylase domain and Domain B. The interdomain region that connects the glycosylase domain and Domain B is underlined for reference.

### DETAILED DESCRIPTION

The systems described herein comprise compositions that modulate gene expression, e.g., by modifying DNA.

The systems described herein comprise compositions that modulate chromatin topology or chromatin structure, e.g., by modifying DNA.

In some aspects, the present disclosure provides a system comprising a first composition comprising a first component comprising a first DNA targeting moiety which binds to a first target DNA site, operably linked to a first incomplete effector moiety, and a second composition comprising a second component comprising a second DNA targeting moiety which binds to a second target DNA site adjacent to the first target site, operably linked to a second incomplete effector moiety, wherein each of the first and the second component interacts with one another to provide an effector activity at or near the target site.

In some embodiments, the system modulates transcription of a gene, e.g., activates or represses transcription, e.g., induces epigenetic changes to chromatin.

Technologies of the present disclosure may include compositions, as described herein, which are comprised of at least two separate fragments (i.e. a first fragment (e.g. a targeting moiety) and a second fragment (e.g. an incomplete effector moiety)), wherein co-localization of the two fragments in three dimensional space permits assembly or reconstitution of an active effector moiety. In some embodiments, an effector moiety modulates a particular activity. In some embodiments, co-localization of the first and second fragments achieves assembly or reconstitution of effector moiety comparable to that observed with an intact effector moiety (e.g., separate from any targeting moiety and/or provided as a discrete chemical entity).

### Incomplete Effector Moieties

In some embodiments, a system comprises at least two incomplete effector moieties (i.e., fragments, elements of a complete effector moiety) as described herein.

In some embodiments, the present disclosure provides technologies for delivering (e.g., providing to and/or causing expression in, e.g., in a functional state or form) both (or all) fragments of a particular composition or system to a cell or cell population. In some embodiments, different fragments may be delivered separately; in some embodiments, two or more fragments may be delivered together (e.g., at a particular point in time and/or via a single route or administration). As used herein, the term “deliver” means providing technologies of the present disclosure to a cell or population of cells. In some embodiments, delivery of systems described herein occurs via administration to a patient (wherein a cell or population of cells exists within the patient). In some embodiments, delivery occurs in vitro, ex vitro and/or in vivo. In some embodiments, delivery occurs via contacting a cell or cells with technologies as provided herein.

In some embodiments, the present disclosure provides a system that comprises and/or delivers two or more compositions as described herein. In some embodiments, such a system comprises a plurality of separate compositions (e.g., distinct compositions, which each may, for example, be formulated as one or a plurality of dosage forms, that each comprise and/or deliver a single modulating entity fragment).

Some aspects of the present disclosure provide split-effector systems to modify DNA. When separate from one another, the effector fragments or incomplete effector moieties do not display 100% reference effector activity. For example, in some embodiments, when physically proximate, two or more incomplete effector moieties interact, thereby substantially reconstituting enough of an effector protein from which they were derived such that effector activity is restored (e.g., restored at least 50%, 60%, 70%, 80%, 85%, 90%, 95% or more). In some embodiments, two or more incomplete effector moieties interact to provide effector activity. In some such embodiments, incomplete effector moieties may be the same or different from one another (e.g. two copies of the same molecule, or two distinct molecules (e.g. an N-terminal portion of a particular effector or nucleic acids encoding it and, e.g. a C-terminal portion of a particular effector or nucleic acids encoding it, etc.). Such systems may be generated from DNA or chromatin modifying effectors, and variants thereof, and are useful for modulating gene expression in living cells, tissues or subjects (e.g., mammals, e.g., human or non-human subjects), in cell lysates, and/or in vitro formats.

In some embodiments, effector proteins are split within sequences between domains, such as between structural domains. In a design of a simple, dual-incomplete effector system, an effector protein may be split into at least two incomplete effector moieties at any location or portion in the effector protein that is between contiguous domains, such as structural motifs, in order to generate a first incomplete effector moiety corresponding to a first set of contiguous structural motifs, and a second incomplete effector moiety corresponding to a second set of contiguous structural motifs.

In some embodiments, an effector protein is not bifurcated when split into at least two incomplete effector moieties.

In some embodiments, an effector protein is split such that a first incomplete effector moiety comprises an N-terminal region (or nucleic acids encoding such a region) and a second incomplete effector moiety comprises a C-terminal region (or nucleic acids encoding such a region). In some embodiments, an N- and/or C-terminal region does not or is not comprised of all amino acids (or nucleic acids encoding them) that one of skill in the art would understand to be the complete N- and/or C-terminal region of a particular effector protein.

In some embodiments, an effector protein is a protein (or nucleic acids encoding it) normally found in a particular cell and/or organism. In some embodiments, at least two incomplete effector protein fragments (or nucleic acids encoding them) reconstitute activity similar or substantially similar to the full-length effector protein.

In some embodiments, an effector protein is not or does not comprise an effector protein that is itself lethal to a cell (e.g. diphtheria toxin, ricin, etc.).

In some embodiments, an effector protein is a protein that is endogenous to a cell(s) and/or organism.

In some embodiments, an effector protein is not or does not comprise an exogenous protein (e.g. diphtheria toxin, ricin, etc.).

In some embodiments, a targeted genomic location is or comprises one or more modified nucleic acids (e.g. methylated nucleic acids, etc.).

In some embodiments, an incomplete effector moiety comprises between about 10%-20%, 20%-30%, 30%-40%, 40%-50%, 50%-60%, 60%-70%, 70%-75%, 75%-80%, 80%-85%, 85%-90%, 90%-95%, 95%-99%, or any percentage therebetween of amino acids of a given effector protein. An incomplete effector moiety may comprise a fragment or a variant of a particular effector protein.

Incomplete effector moieties may have a length from about 5 to about 200 amino acids, about 15 to about 150 amino acids, about 20 to about 125 amino acids, about 25 to about 100 amino acids, or any range therebetween.

In some embodiments, an incomplete effector moiety is conditionally inactive. An incomplete effector moiety can have less than 90%, less than 80%, less than 70%, less than 60%, less than 50%, less than 40%, less than 30%, less than 20%, less than 10%, less than 5%, or less than 1% of effector activity of a provided effector protein (e.g., wild-type). An incomplete effector moiety can have no substantial effector activity.

In some embodiments, incomplete effector moieties are derived from an epigenetic modifying agent. Epigenetic modifying agents useful in methods and compositions as provided herein include agents that affect, e.g., DNA methylation, and RNA-associated silencing.

In some embodiments, methods provided herein involve sequence-specific targeting of an epigenetic enzyme (e.g., an enzyme that generates or removes epigenetic marks, e.g., acetylation and/or methylation). Exemplary epigenetic enzymes, that can be targeted to a DNA sequence with a DNA targeting moiety described herein, include DNA methyltransferases (e.g., DNMT3a, DNMT3b, DNMTL, DRM2), DNA demethylation (e.g., the TET family, DME), histone methyltransferase, deaminase, acetyltransferase, histone deacetylase (e.g., HDAC1, HDAC2, HDAC3), sirtuin 1, 2, 3, 4, 5, 6, or 7, lysine-specific histone demethylase 1 (LSD1), histone-lysine-N-methyltransferase (Setdb1), euchromatic histone-lysine N-methyltransferase 2 (G9a), histone-lysine N-methyltransferase (SUV39H1), enhancer of zeste homolog 2 (EZH2), viral lysine methyltransferase (vSET), histone methyltransferase (SET2), protein-lysine N-methyltransferase (SMYD2), ligases, nucleases (e.g., endonucleases, T7, Cpf1, Cas9, zinc finger nuclease), phosphatases (e.g., alkaline phosphatases), recombinases (e.g., Cre), transposases (Tn3, Tn5, Sleeping Beauty), polynucleotide kinases (e.g., T4), enzymes with a role in DNA repair (e.g., RecA, N-glycosylase, AP-lyase), enzymes with a role in DNA demethylation (e.g., the TET family enzymes catalyze oxidation of 5-methylcytosine to 5-hydroxymethylcytosine and higher oxidative derivatives). Examples of such epigenetic modifying agents are described, e.g., in de Groote et al., Nuc. Acids Res. (2012):1-18; in Table 1 of Park et al., Genome Biol., 2016, 17:183; and Table 1 of Morera et al., Clin. Epigenet., 2016, 8:57. Examples of plant proteins involved in methylation and demethylation and epigenetic modification can be found, for example, in Law et al., Nat. Rev. Genet., 2010, 11:204-220; Baumbusch et al., Nucl. Acids Res., 2001, 29:4319-4333; and Du et al., Nat. Rev. Mol. Cell Biol., 2015, 16:519-532. In some embodiments, incomplete effector moieties are derived from e.g., Cbp/p300, SIRT1-6, MLL1, SET, ASH, SUV39H, G9a, HP1, EZH2, LSD1.

In some embodiments, an epigenetic enzyme is not a methyltransferase.

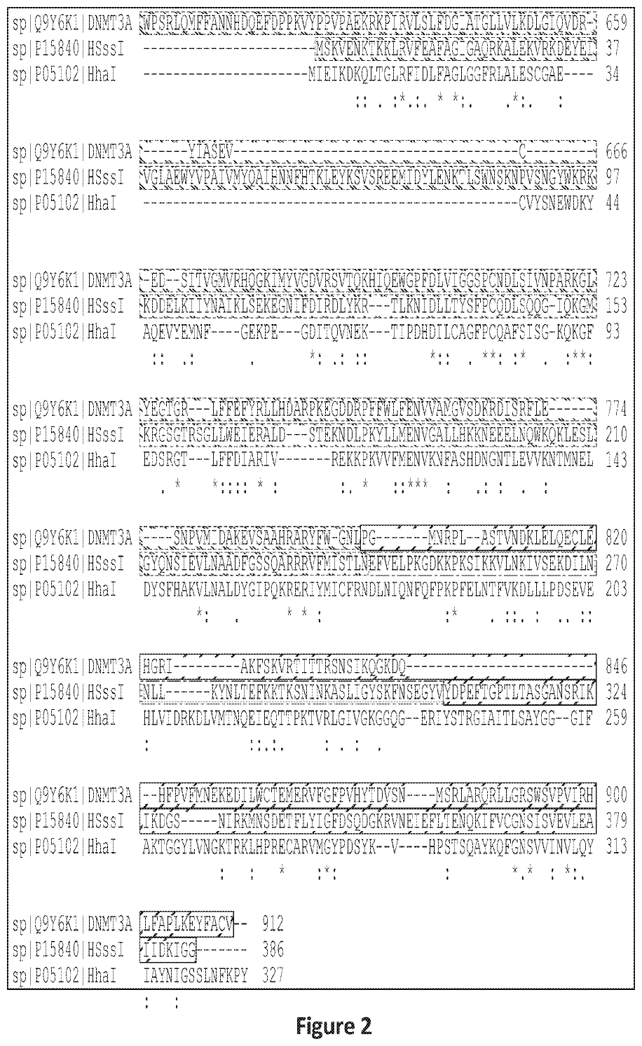

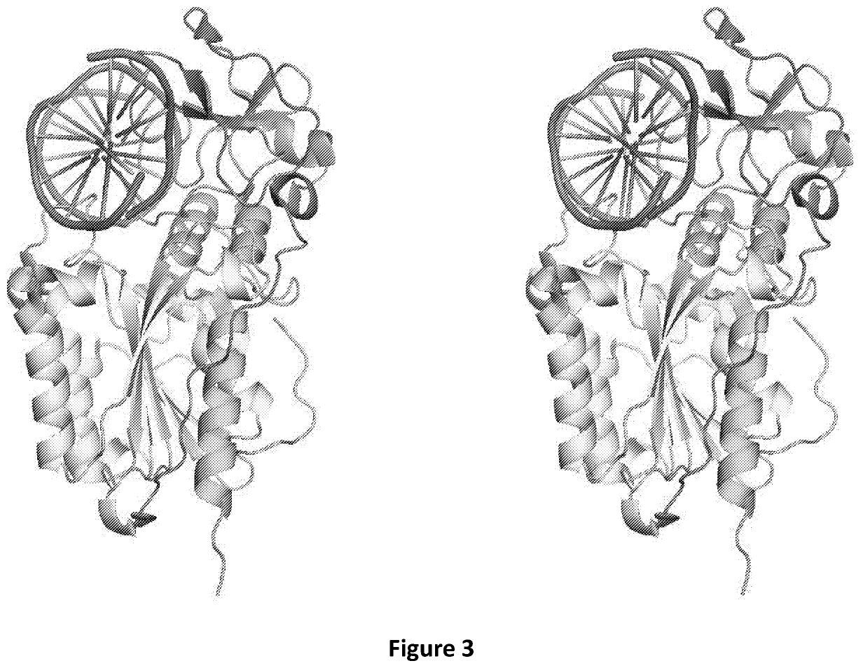

In some embodiments, an incomplete effector moiety is derived from a SET protein or SET domain protein. Some examples of SET domain proteins can be found in Table 1 of Baumbusch, et al., Nucl Ac Res, 2001, 29:4319-4333. Some examples of proteins involved in DNA methylation and demethylation can be found in Table 1 of Law, et al., Nat Rev Genet, 2010, 11:204-220. Protein domain information for select effectors can be found in Table 1 of Law, et al., Nat Rev Genet, 2010, 11:204-220, as well as, FIGS. 2-3 of Nat Rev Mol Cell Biol, 2015, 16:519-532.

In some embodiments, an incomplete effector moiety is derived from a Cas protein. Specific examples of Cas proteins include class II systems including Cas1, Cas2, Cas3, Cas4, Cas5, Cas6, Cas7, Cas8, Cas9, Cas10, Cpf1, C2C1, or C2C3. In some embodiments, the incomplete effector moiety is derived from a Cas protein variant, e.g., Cas9 ribonucleoprotein complexes, see Staahl et al., Nat. Biotech., 2017, doi:10.1038/nbt.3806.

In some embodiments, interaction of two incomplete effector moieties recapitulates an effector activity, e.g., enzymatic activity, regulation of gene expression, regulation of signaling, and/or regulation of cellular or organ function. Effector activities may also include binding regulatory proteins to modulate activity of the regulator, such as transcription or translation. Effector activities also may include activator or inhibitor functions. Effector activities may also include modulating transcript stability/degradation. In some embodiments, interaction of two incomplete effector moieties reconstitutes the effector domain of the full-length effector protein from which they were derived, thereby restoring effector activity. In some embodiments, interaction of two incomplete effector moieties confers the same, substantially the same, or similar function as the full-length effector protein, regardless of whether the complete effector domain is reconstituted.

In some embodiments, complete effector activity induces homologous recombination by generating one or more double-stranded DNA breaks in the target nucleotide sequence, followed by repair of the break(s) using a homologous recombination mechanism (“homology-directed repair”).

In some embodiments, a system comprises a nucleic acid encoding one or more incomplete effector moieties described herein. Accordingly, in some embodiments, a nucleic acid encoding such incomplete effector moiety(ies) is administered to a subject in need thereof and the incomplete effector moiety is expressed from the nucleic acid after administration to the subject. In some embodiments, nucleic acids encoding such incomplete effector moiety(ies) is/are administered to a subject in need thereof. In some embodiments, the incomplete effector moiety is expressed from the nucleic acid before administration to the subject. In some embodiments, the incomplete effector moiety is expressed from the nucleic acid after administration to the subject.

An effector may be a peptidic effector, e.g., a protein such as an enzyme. Alternatively, an effector may be a non-peptidic, e.g., a chemical effector, such as DNA intercalating agents for targeted mutagenesis and deaminating molecules.

### DNA Targeting Moieties

In some embodiments, a system comprises at least two incomplete effector moieties. In some embodiments, such a system further comprises one or more DNA targeting moieties as described herein.

In some embodiments, a DNA targeting moiety targets one or more target DNA sequences, e.g., a target DNA site. In some embodiments, a DNA targeting moiety binds a promoter to alter expression of a gene. In some embodiments, a DNA targeting moiety targets one or more DNA sites adjacent to a target DNA site, e.g., a methylation site in a promoter or a gene regulation sequence.

In some embodiments, a targeting moiety recruits one or more incomplete effector moieties to the target DNA site. In some such embodiments, a targeting moiety interacts with a DNA sequence at or near the target DNA site and with an incomplete effector moiety. In some embodiments, when multiple incomplete effector moieties are recruited to a target DNA site, incomplete effector moieties interact to provide an effector activity at or near the target site.

A DNA targeting moiety may bind a target DNA sequence and recruit one or more incomplete effector moieties to modulate transcription, in a human cell, of a gene adjacent to the target DNA sequence. In some embodiments, a target DNA sequence is adjacent to a gene regulation site, e.g. binding site for an epigenetic modifying enzyme, an alternative splicing site, and a binding site for a non-translated RNA.

In some embodiments, a DNA targeting moiety is a nucleic acid sequence, a protein, protein fusion, or an analog thereof.

Nucleic Acids

In some embodiments, a DNA targeting moiety is a nucleic acid sequence selected from DNA, RNA, or an analog thereof. The DNA targeting moiety can be, but is not limited to, DNA, RNA, and artificial nucleic acids. In some embodiments, a nucleic acid sequence includes, but is not limited to, genomic DNA, cDNA, tRNA, mRNA, rRNA, modified RNA, miRNA, gRNA, and siRNA or other RNAi molecule.

In some embodiments, DNA targeting moieties may comprise a sequence substantially complementary, or fully complementary, to all or some (e.g. a fragment) of a target gene. DNA targeting moieties may complement sequences at boundaries between one or more introns and exons to prevent maturation of newly-generated nuclear RNA transcripts of specific genes into mRNA for transcription. In some embodiments, DNA targeting moieties complementary to specific genes can hybridize with mRNA for a target gene and prevent its translation. In some embodiments, an antisense molecule can be DNA, RNA, or a derivative or hybrid thereof. Examples of such derivative molecules may include, but are not limited to, peptide nucleic acid (PNA) and phosphorothioate-based molecules such as deoxyribonucleic guanidine (DNG) or ribonucleic guanidine (RNG).

In some embodiments, a DNA targeting moiety comprises nucleic acid sequence at least 50%, at least 60%, at least 70%, at least 80%, at least 90%, at least 95%, at least 96%, at least 97%, at least 98%, at least 99% complementary to a sequence adjacent to a target DNA site, e.g., a gene regulation site. In some embodiments, a nucleic acid sequence comprises a sequence at least 50%, at least 60%, at least 70%, at least 80%, at least 90%, at least 95%, at least 96%, at least 97%, at least 98%, at least 99% complementary to a promoter, enhancer, silencer, or repressor of a gene. Degree of complementary or identity to a sequence of a target DNA should be at least 75%, at least 80%, at least 85%, at least 90%, or at least 95% or more.

Length of DNA targeting moieties that hybridize to a target gene may be around 10 nucleotides, between about 15 or 30 nucleotides, or about 15, 16, 17, 18, 19, 20, 21, 22, 23, 24, 25, 26, 27, 28, 29, 30 or more nucleotides. In some embodiments, a DNA targeting moiety has a length from about 2 to about 5000 nts, about 10 to about 100 nts, about 50 to about 150 nts, about 100 to about 200 nts, about 150 to about 250 nts, about 200 to about 300 nts, about 250 to about 350 nts, about 300 to about 500 nts, about 10 to about 1000 nts, about 50 to about 1000 nts, about 100 to about 1000 nts, about 1000 to about 2000 nts, about 2000 to about 3000 nts, about 3000 to about 4000 nts, about 4000 to about 5000 nts, or any range therebetween.

Some examples of nucleic acids include, but are not limited to, a nucleic acid that hybridizes to an endogenous gene (e.g., gRNA as described herein elsewhere), nucleic acid that hybridizes to an exogenous nucleic acid such as a viral DNA or RNA, nucleic acid that hybridizes to an RNA, nucleic acid that interferes with gene transcription, nucleic acid that interferes with RNA translation, nucleic acid that stabilizes RNA or destabilizes RNA such as through targeting for degradation, nucleic acid that interferes with a DNA or RNA binding factor through interference of its expression or its function, nucleic acid that is linked to a intracellular protein and modulates its function, and nucleic acid that is linked to an intracellular protein complex and modulates its function.

In some embodiments, a DNA targeting moiety comprises RNA or RNA-like structures typically containing 5-150 base pairs (such as about 15-50 base pairs) and having a nucleobase sequence identical (complementary) or nearly identical (substantially complementary) to a coding sequence in an expressed target gene within a cell. RNA molecules include, but are not limited to: short interfering RNAs (siRNAs), double-strand RNAs (dsRNA), micro RNAs (miRNAs), short hairpin RNAs (shRNA), meroduplexes, and dicer substrates (U.S. Pat. Nos. 8,084,599 8,349,809 and 8,513,207).

In some embodiments, a DNA targeting moiety comprises a nucleic acid sequence, e.g., a guide RNA (gRNA). In some embodiments, a DNA targeting moiety comprises a guide RNA or nucleic acid encoding the guide RNA. A gRNA short synthetic RNA composed of a “scaffold” sequence necessary for binding to an incomplete effector moiety and a user-defined ˜20 nucleotide targeting sequence for a genomic target. In practice, guide RNA sequences are generally designed to have a length of between 17-24 nucleotides (e.g., 19, 20, or 21 nucleotides) and complementary to a targeted nucleic acid sequence. Custom gRNA generators and algorithms are available commercially for use in designing effective guide RNAs. Gene editing has also been achieved using a chimeric “single guide RNA” (“sgRNA”), an engineered (synthetic) single RNA molecule that mimics a naturally occurring crRNA-tracrRNA complex and contains both a tracrRNA (for binding the nuclease) and at least one crRNA (to guide the nuclease to the sequence targeted for editing). Chemically modified sgRNAs have also been demonstrated to be effective in genome editing; see, for example, Hendel et al. (2015) Nature Biotechnol., 985-991.

In some embodiments, a DNA targeting moiety comprises a gRNA that recognizes specific DNA sequences (e.g., sequences adjacent to or within a promoter, enhancer, silencer, or repressor of a gene). In some such embodiments, the gRNA is combined with one or more peptides, e.g., S-adenosyl methionine (SAM), that acts as a substrate for methyl group transfers.

In some embodiments, a DNA targeting moiety may also comprise nucleotides not directly involved in pairing to the target DNA site and/or the incomplete effector moiety, i.e. typically unpaired, overhanging nucleotides. In some embodiments, a DNA targeting moiety may contain 3′ and/or 5′ overhangs of about 1-5 bases independently on the 5′ or the 3′ end. In one embodiment, both the 3′ and 5′ has an overhang. In some embodiments, the 3′ end of a DNA targeting moiety has an overhang. In some embodiments, the 5′ end of a DNA targeting moiety has an overhang. In some embodiments, one or more nucleotides in an overhang contains a thiophosphate, phosphorothioate, deoxynucleotide inverted (3′ to 3′ linked) nucleotide or is a modified ribonucleotide or deoxynucleotide.

In some embodiments, a DNA targeting moiety may include nucleosides, e.g., purines or pyrimidines, e.g., adenine, cytosine, guanine, thymine and uracil. In some embodiments, a DNA targeting moiety described herein has one or more modified nucleosides or nucleotides. Such modifications are known and are described, e.g., in WO 2012/019168. Additional modifications are described, e.g., in WO2015038892; WO2015038892; WO2015089511; WO2015196130; WO2015196118 and WO2015196128A2.

In some embodiments, a DNA targeting moiety includes one or more nucleoside analogs. In some such embodiments, a nucleoside analog may include, but is not limited to, a nucleoside analog, such as 5-fluorouracil; 5-bromouracil, 5-chlorouracil, 5-iodouracil, hypoxanthine, xanthine, 4-acetylcytosine, 4-methylbenzimidazole, 5-(carboxyhydroxylmethyl) uracil, 5-carboxymethylaminomethyl-2-thiouridine, 5-carboxymethylaminomethyluracil, dihydrouracil, dihydrouridine, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, 2,6-diaminopurine, 3-nitropyrrole, inosine, thiouridine, queuosine, wyosine, diaminopurine, isoguanine, isocytosine, diaminopyrimidine, 2,4-difluorotoluene, isoquinoline, pyrrolo[2,3-β]pyridine, and any others that can base pair with a purine or a pyrimidine side chain.

Chimeric enzymes for synthesizing capped RNA molecules (e.g., modified mRNA) which may include at least one chemical modification are described in WO2014028429.

In some embodiments, a DNA targeting moiety described herein comprising a modified mRNA may have one or more terminal modifications, e.g., a 5′Cap structure and/or a poly-A tail (e.g., of between 100-200 nucleotides in length). A 5′Cap structure may be selected from the group consisting of CapO, Cap1, ARCA, inosine, N1-methyl-guanosine, 2′fluoro-guanosine, 7-deaza-guanosine, 8-oxo-guanosine, 2-amino-guanosine, LNA-guanosine, and 2-azido-guanosine. In some embodiments, modified RNAs may also contain a 5′ UTR comprising at least one Kozak sequence, and a 3′ UTR. Such modifications are known and are described, e.g., in WO2012135805 and WO2013052523. Additional terminal modifications are described, e.g., in WO2014164253 and WO2016011306. WO2012045075 and WO2014093924.

In some embodiments, a DNA targeting moiety as described herein, comprising a modified mRNA, may be cyclized or concatemerized. In some such embodiments, cyclization or concatemerization may generate a translation competent molecule to assist interactions between poly-A binding proteins and 5′-end binding proteins. Mechanism(s) of cyclization or concatemerization may occur through at least 3 different routes: 1) chemical; 2) enzymatic; and/or 3) ribozyme catalyzed. Newly formed 5′-/3′-linkages may be intramolecular or intermolecular. Such modifications are described, e.g., in WO2013151736.

Methods of making and purifying modified RNAs are known and disclosed in the art. For example, modified RNAs are made using only in vitro transcription (IVT) enzymatic synthesis. Methods of making IVT polynucleotides are known in the art and are described in WO2013151666, WO2013151668, WO2013151663, WO2013151669, WO2013151670, WO2013151664, WO2013151665, WO2013151671, WO2013151672, WO2013151667 and WO2013151736.S Methods of purification include purifying an RNA transcript comprising a polyA tail by contacting the sample with a surface linked to a plurality of thymidines or derivatives thereof and/or a plurality of uracils or derivatives thereof (polyT/U) under conditions such that the RNA transcript binds to the surface and eluting the purified RNA transcript from the surface (WO2014152031); using ion (e.g., anion) exchange chromatography that allows for separation of longer RNAs up to 10,000 nucleotides in length via a scalable method (WO2014144767); and subjecting a modified RMNA sample to DNAse treatment (WO2014152030).

DNA Binding Domains

In some embodiments, a DNA targeting moiety comprises a DNA-binding domain. In some embodiments, DNA-binding proteins have distinct structural motifs that play a key role in binding DNA.

In some embodiments, a DNA targeting moiety comprises a helix-turn-helix motif to interact with a target DNA site. In some embodiments, a helix-turn-helix motif is a common DNA recognition motif in repressor proteins. In some such embodiments, a motif comprises two helices, one of which recognizes DNA (aka recognition helix), with side chains providing specificity of binding. In some embodiments, more than one protein may compete to bind to the same DNA sequence or may recognize the same DNA fragment. In some such embodiments, such proteins may differ in their affinities for the same DNA sequence or DNA conformation. In some such embodiments, affinity for a given DNA sequence or confirmation is governed by H-bonds, salt bridges, and/or Van der Waals interactions.

In some embodiments, DNA-binding proteins with an HhH structural motif may be involved in non-sequence-specific DNA binding that occurs via formation of hydrogen bonds between protein backbone nitrogens and DNA phosphate groups.

In some embodiments, a DNA targeting moiety comprises a leucine zipper domain. In some such embodiments, a leucine zipper motif includes two amphipathic helices, one from each subunit, interacting with each other resulting in a left handed coiled-coil super secondary structure. A leucine zipper is an interdigitation of regularly spaced leucine residues in one helix with leucines from an adjacent helix. In some embodiments, helices involved in leucine zippers exhibit a heptad sequence (abcdefg) where residues a and dare hydrophobic and all others residues are hydrophilic. Leucine zipper motifs can mediate either homo- or heterodimer formation.

In some embodiments, a DNA targeting moiety comprises a Zn-finger domain, where a Zn⁺⁺ ion is coordinated by 2 Cys and 2 His residues. Each Zn-finger interacts in a conformationally identical manner with successive triple base pair segments in the major groove of the double helix of the DNA with which it interacts. In some embodiments, protein-DNA interaction is determined by two factors: (i) H-bonding interaction between α-helix and DNA segment, mostly between Arg residues and Guanine bases; and (ii) H-bonding interaction with the DNA phosphate backbone, mostly with Arg and His. In some embodiments, an alternative Zn-finger motif chelates Zn⁺⁺ with 6 Cys.

In some embodiments, a DNA targeting moiety comprises a TATA box binding protein (TBP) domain. Structure of TBP shows two a/P structural domains of 89-90 amino acids. The C-terminal or core region binds with high affinity to a TATA consensus sequence recognizing minor groove determinants and promoting DNA bending. TBP resemble a molecular saddle. The binding side is lined with the central 8 strands of the 10-stranded anti-parallel β-sheet. The upper surface contains four α-helices and binds to various components of the transcription machinery.

In some embodiments, a DNA targeting moiety comprises amino acids with basic residues, such as Lysine, Arginine, Histidine, Asparagine and Glutamine, to interact with adenine of A: T base pairs, and guanine of G: C base pairs. NH2 and X═O groups of base pairs can form hydrogen bonds with amino acid residues of Glutamine, Asparagine, Arginine, and Lysine. DNA provides base specificity in the form of nitrogen bases.

In some embodiments, a DNA targeting moiety may bind a target DNA sequence and recruit one or more incomplete effector moieties to modulate transcription, in a human cell, of a gene adjacent to the target DNA sequence. In some embodiments, a target DNA sequence is adjacent to a gene regulation site, e.g. binding site for an epigenetic modifying enzyme, an alternative splicing site, and a binding site for a non-translated RNA.

In some embodiments, a system comprises two or more DNA targeting moieties (a first DNA targeting moiety and a second DNA targeting moiety) that are not identical. In some embodiments, a first DNA targeting moiety recruits a first incomplete effector moiety to a target DNA site. In some embodiments, a second DNA targeting moiety recruits a second incomplete effector moiety to a site adjacent to the target DNA site. When individual DNA targeting moieties interact with their respective target DNA sites, incomplete effector moieties are brought within close proximity to each other. In some embodiments, two incomplete effector moieties interact to provide an effector activity at or near a target site.

In some embodiments, a DNA targeting moiety targets a DNA sequence adjacent to a target DNA site. In some such embodiments, sequences adjacent to one another may be contiguous or non-contiguous. In some embodiments, sequences adjacent to one another are not contiguous. In some embodiments, sequences adjacent to one another are not non-contiguous.

In some embodiments, a DNA targeting moiety targets a DNA site adjacent to, e.g., within 2-1000 nucleotides, one or more gene regulation sites, e.g., DNA methylation sites. In some embodiments, a target DNA site may be adjacent to a gene regulation site, e.g., about at least 1, at least 5, at least 10, at least 20, at least 30, at least 40, at least 50, at least 100, at least 150, at least 200, at least 300, at least 400, at least 500, at least 600, at least 750, or at least 1000 nucleotides from the gene regulation site. In some embodiments, a target DNA site may be at least 10, at least 20, at least 30, at least 40, at least 50, at least 60, at least 70, at least 80, at least 90, or at least 100 nucleotides from a gene regulation site.

In some embodiments, a DNA targeting moiety targets a DNA sequence adjacent to, e.g., within structural proximity, e.g., two or three-dimensional proximity, to a target DNA site. In some embodiments, a DNA targeting moiety targets a DNA sequence in a chromatin structure, e.g., a helix, nucleosome, fiber, within structural proximity to a target DNA site, e.g., 1, 2, 3, 4, 5, 6, 7, 8, etc. helical turns away. In some embodiments, sequences adjacent to one another may be contiguous or non-contiguous.

In some embodiments, a DNA targeting moiety targets one or more nucleotides, e.g., such as through a DNA binding domain of a zinc finger domain, TALEN, caspase enzyme, recombinase, transposase, etc.

In some embodiments, a DNA targeting moieties recruit one or more incomplete effector moieties to a DNA target site to provide an effector activity that modulates transcription, in a human cell, of a gene. In some embodiments, an effector activity may alter a target site through a substitution, addition, or deletion of one or more nucleotides. In some embodiments, an effector activity may alter at least one of a binding site for a gene regulation protein, e.g. an epigenetic modifying agent, e.g., DNA methyltransferases (e.g., DNMT3a, DNMT3b, DNMTL, DRM2), DNA demethylation (e.g., the TET family, DME), histone methyltransferase, deaminase, acetyltransferase, histone deacetylase, ligases, nucleases (e.g., endonucleases, T7, Cpf1, Cas9, zinc finger nuclease), phosphatases (e.g., alkaline phosphatases), recombinases (e.g., Cre), transposases (Tn3, Tn5, Sleeping Beauty), polynucleotide kinases (e.g., T4), enzymes with a role in DNA repair (e.g., RecA, N-glycosylase, AP-lyase), enzymes with a role in DNA demethylation (e.g., the TET family enzymes catalyze oxidation of 5-methylcytosine to 5-hydroxymethylcytosine and higher oxidative derivatives).

In some embodiments, a DNA targeting moiety is a nucleic acid that encodes a desired DNA targeting moiety and is provided to a cell or subject; examples of suitable nucleic acids include single-stranded DNA, double-stranded DNA, RNA, and analogs thereof.

In some embodiments, a DNA targeting moiety targets one or more nucleotides, e.g., such as through a DNA binding domain. In some embodiments, a DNA targeting moiety is derived from a transcription factor. In some embodiments, a DNA targeting moiety is a fragment or a variant of a transcription factor. In some embodiments, a DNA targeting moiety comprises a DNA binding domain from a transcription factor, and an incomplete effector moiety comprises a fragment or a variant of an effector domain from the same transcription factor comprised by the DNA targeting moiety. In some embodiments, upon interaction of complementary incomplete effector moieties, transcription is activated or repressed.

### Compositions

In some aspects, the present disclosure provides systems and methods of modulating expression of a gene by administering the components described herein. In some aspects, a system is described comprising one or more compositions. Each composition comprises one or more components, wherein each component comprises a DNA targeting moiety as described herein operably linked to an incomplete effector moiety as described herein. Multiple components interact to provide an effector activity at or near the target site.

In some aspects, the present disclosure provides a system comprising a first composition comprising a first component comprising a first DNA targeting moiety capable of binding to a first target DNA site, operably linked to a first incomplete effector moiety, and a second composition comprising a second component comprising a second DNA targeting moiety capable of binding to a second target DNA site adjacent to the first target site, operably linked to a second incomplete effector moiety, wherein the first and second component are capable of interacting to provide an effector activity at or near the target site.

In some embodiments, compositions of the present disclosure are operably linked.

In some embodiments, a composition comprises a component that binds a nucleic acid sequence adjacent to the target site. In some embodiments, a composition comprises a nucleic acid encoding one or more components described herein. Accordingly, in some embodiments, a nucleic acid encoding an incomplete effector moiety and/or a DNA targeting moiety is administered to a subject in need thereof and either one or both of the incomplete effector moiety and the DNA targeting moiety is expressed from the nucleic acid that encodes them.

In some aspects, the present disclosure includes a pharmaceutical composition comprising one or more components described herein. In some embodiments, more than one composition is formulated in a single pharmaceutical composition.

In some aspects, the present disclosure provides a system comprising: a) a first nucleic acid sequence encoding a first incomplete effector moiety; b) a first DNA targeting moiety that interacts with the first incomplete effector moiety and binds to a first target DNA site; c) a second nucleic acid sequence encoding a second incomplete effector moiety; and d) a second DNA targeting moiety that interacts with the second incomplete effector moiety and binds to a second target DNA site adjacent to the first target site, wherein the first and second incomplete effector moieties interact to provide an effector activity at or near the target site.

Membrane Penetrating Moieties

In some embodiments, compositions or components thereof, as described herein, may be linked to one or more membrane penetrating moieties to carry one or more compositions or components thereof into cells or across a membrane, e.g., cell or nuclear membrane. As will be appreciated by one of skill in the art, membrane penetrating moieties that are capable of facilitating transport of substances across a membrane include, but are not limited to, cell-penetrating peptides (CPPs)(see, e.g., U.S. Pat. No. 8,603,966), fusion peptides for plant intracellular delivery (see, e.g., Ng et al., PLoS One, 2016, 11:e0154081), protein transduction domains, Trojan peptides, and membrane translocation signals (MTS) (see, e.g., Tung et al., Advanced Drug Delivery Reviews 55:281-294 (2003)). Some MTS are rich in amino acids with positively charged side chains such as arginine.

In some embodiments, membrane penetrating moieties are able to induce membrane penetration of a component and allow macromolecular translocation within cells of multiple tissues in vivo upon systemic administration. A membrane penetrating moiety may also refer to a peptide which, when brought into contact with a cell under appropriate conditions, passes from the external environment of the cell into the intracellular environment (which includes, e.g. the cytoplasm, organelles such as mitochondria, or cell nucleus), in conditions significantly greater than passive diffusion.

In some embodiments, compositions or their components transported across a membrane may be reversibly or irreversibly linked to a membrane penetrating moiety. Optionally, in some embodiments, a linker can be used to link a component and a membrane penetrating moiety. Any linker described elsewhere herein may be suitable.

Linkers

In some embodiments, an incomplete effector moiety (e.g., a fragment of a DNA methyltransferase, histone methyltransferase, deaminase, acetyltransferase, histone deacetylase, ligase, nuclease, phosphatase, recombinase, transposase, polynucleotide kinase, enzyme with a role in DNA repair, enzyme with a role in DNA demethylation) is linked to a DNA targeting moiety (e.g., a gRNA or DNA binding domain).

In some embodiments, an incomplete effector moiety described herein can be linked to a DNA targeting moiety by employing standard ligation techniques. Such methods include, general native chemical ligation strategies (Siman, P. and Brik, A. Org. Biomol. Chem. 2012, 10:5684-5697; Kent, S. B. H. Chem. Soc. Rev. 2009, 38:338-351; and Hackenberger, C. P. R. and Schwarzer, D. Angew. Chem., Int. Ed. 2008, 47:10030-10074), click modification protocols (Tasdelen, M. A.; Yagci, Y. Angew. Chem., Int. Ed. 2013, 52:5930-5938; Palomo, J. M. Org. Biomol. Chem. 2012, 10:9309-9318; Eldijk, M. B.; van Hest, J. C. M. Angew. Chem., Int. Ed. 2011, 50:8806-8827; and Lallana, E.; Riguera, R.; Fernandez-Megia, E. Angew. Chem., Int. Ed. 2011, 50:8794-8804), and bioorthogonal reactions (King, M.; Wagner, A. Bioconjugate Chem. 2014, 25:825-839; Lang, K.; Chin, J. W. Chem. Rev. 2014, 114:4764-4806; Patterson, D. M.; Nazarova, L. A.; Prescher, J. A. ACS Chem. Biol. 2014, 9:592-605; Lang, K.; Chin, J. W. ACS Chem. Biol. 2014, 9:16-20; Takaoka, Y.; Ojida, A.; Hamachi, I. Angew. Chem., Int. Ed. 2013, 52:4088-4106; Debets, M. F.; van Hest, J. C. M.; Rutjes, F. P. J. T. Org. Biomol. Chem. 2013, 11:6439-6455; and Ramil, C. P.; Lin, Q. Chem. Commun. 2013, 49:11007-11022).

In some embodiments, an incomplete effector moiety is linked to a DNA targeting moiety through a phosphoamide bond between the polypeptide and internucleotide phosphate groups, e.g., a phospho-triester between a hydroxy amino acid residue in the incomplete effector moiety and an internucleotide phosphate.

In some embodiments, components described herein may also include a linker. In some embodiments, an incomplete effector moiety is operably linked to a DNA targeting moiety. In some embodiments, a linker may be a chemical bond, e.g., one or more covalent bonds or non-covalent bonds. In some embodiments, a linker is a peptide linker. In some such embodiments, a linker may be between 2-30 amino acids, or longer. In some embodiments, a linker includes flexible, rigid or cleavable linkers described herein.

As will be appreciated by one of ordinary skill in the art, commonly used flexible linkers have sequences consisting primarily of stretches of Gly and Ser residues (“GS” linker). In some embodiments, flexible linkers may be useful for joining domains that require a certain degree of movement or interaction and may include small, non-polar (e.g. Gly) or polar (e.g. Ser or Thr) amino acids. In some embodiments, incorporation of Ser or Thr can also maintain stability of a particular linker in aqueous solutions by forming hydrogen bonds with the water molecules, and therefore reduce unfavorable interactions between the linker and protein moieties.

As will be understood by one of skill in the art, rigid linkers are useful to keep a fixed distance between domains and to maintain their independent functions. In some embodiments, rigid linkers may also be useful when a spatial separation of the domains is critical to preserve the stability or bioactivity of one or more components in the fusion. In some embodiments, rigid linkers may have an alpha helix-structure or Pro-rich sequence, (XP)ₙ, with X designating any amino acid, preferably Ala, Lys, or Glu.

In some embodiments, cleavable linkers may release free functional domains in vivo. In some embodiments, linkers may be cleaved under specific conditions, such as presence of reducing reagents or proteases. For example, in some embodiments, e.g. in vivo, cleavable linkers may utilize reversibility of a disulfide bond. By way of non-limiting example, in some embodiments, a thrombin-sensitive sequence (e.g., PRS) is located between two Cys residues. In vitro thrombin treatment of CPRSC results in cleavage of this thrombin-sensitive sequence, while the reversible disulfide linkage remains intact. As will be appreciated by one of skill in the art, such linkers are known and described, e.g., in Chen et al. 2013. Fusion Protein Linkers: Property, Design and Functionality. Adv Drug Deliv Rev. 65(10): 1357-1369. In some embodiments, in vivo cleavage of linkers in fusions may also be carried out by proteases that are expressed in vivo under pathological conditions (e.g. cancer or inflammation), in specific cells or tissues, or constrained within certain cellular compartments. Without wishing to be bound by any particular theory, specificity of many proteases may offer slower cleavage of a linker in constrained compartments.

Examples of linking molecules include a hydrophobic linker, such as a negatively charged sulfonate group; lipids, such as a poly (—CH₂—) hydrocarbon chains, such as polyethylene glycol (PEG) group, unsaturated variants thereof, hydroxylated variants thereof, amidated or otherwise N-containing variants thereof, noncarbon linkers; carbohydrate linkers; phosphodiester linkers, or other molecule capable of covalently linking two or more polypeptides. Non-covalent linkers are also included, such as, e.g., hydrophobic lipid globules to which a polypeptide is linked, for example through a hydrophobic region of the polypeptide or a hydrophobic extension of the polypeptide, such as a series of residues rich in leucine, isoleucine, valine, or perhaps also alanine, phenylalanine, or even tyrosine, methionine, glycine or other hydrophobic residue. In some embodiments, a polypeptide may be linked using charge-based chemistry, such that a positively charged moiety of the polypeptide is linked to a negative charge of another polypeptide or nucleic acid.

Preparation of Components

Methods of making certain components as described herein are routine in the art. See, in general, Smales & James (Eds.), Therapeutic Proteins: Methods and Protocols (Methods in Molecular Biology), Humana Press (2005); and Crommelin, Sindelar & Meibohm (Eds.), Pharmaceutical Biotechnology: Fundamentals and Applications, Springer (2013).

Components of compositions provided by the present disclosure may be biochemically synthesized, e.g., by employing standard solid phase techniques. In some embodiments, such methods include exclusive solid phase synthesis, partial solid phase synthesis methods, fragment condensation, and/or classical solution synthesis. In some such embodiments, these methods can be used when a peptide is relatively short (i.e., 10 kDa) and/or when it cannot be produced by recombinant techniques (i.e., not encoded by a nucleic acid sequence) and therefore involves different chemistry.

As will be known to one of skill in the art, solid phase synthesis procedures are well known and further described, e.g., by John Morrow Stewart and Janis Dillaha Young, Solid Phase Peptide Syntheses, 2nd Ed., Pierce Chemical Company, 1984; and Coin, I., et al., Nature Protocols, 2:3247-3256, 2007.

In some embodiments, such as, e.g. those involving longer peptides, recombinant methods may be used. Methods of making a recombinant therapeutic peptides are routine in the art. See, in general, Smales & James (Eds.), Therapeutic Proteins: Methods and Protocols (Methods in Molecular Biology), Humana Press (2005); and Crommelin, Sindelar & Meibohm (Eds.), Pharmaceutical Biotechnology: Fundamentals and Applications, Springer (2013).

By way of non-limiting example, methods for producing a therapeutic pharmaceutical component involve expression in mammalian cells, although recombinant proteins can also be produced using insect cells, yeast, bacteria, or other cells under the control of appropriate promoters. In some embodiments, mammalian expression vectors may comprise non-transcribed elements such as an origin of replication, a suitable promoter and enhancer, and other 5′ or 3′ flanking non-transcribed sequences, and 5′ or 3′ non-translated sequences such as necessary ribosome binding sites, a polyadenylation site, splice donor and acceptor sites, and termination sequences. In some embodiments, DNA sequences derived from the SV40 viral genome, for example, SV40 origin, early promoter, enhancer, splice, and/or polyadenylation sites may be used to provide certain genetic elements required for expression of a heterologous DNA sequence. Appropriate cloning and expression vectors for use with bacterial, fungal, yeast, and mammalian cellular hosts are described, e.g., in Green & Sambrook, Molecular Cloning: A Laboratory Manual (Fourth Edition), Cold Spring Harbor Laboratory Press (2012).

In some embodiments, such as, e.g. cases where large amounts of components of the presently described compositions are desired, techniques such as, e.g. described by Brian Bray, Nature Reviews Drug Discovery, 2:587-593, 2003; and Weissbach & Weissbach, 1988, Methods for Plant Molecular Biology, Academic Press, NY, Section VIII, pp 421-463, may be used.

In some embodiments, various mammalian cell culture systems can be employed to express and manufacture recombinant protein(s). By way of non-limiting example, mammalian expression systems include CHO, COS, HeLA, HEK293, and BHK cell lines. Processes of host cell culture for production of protein therapeutics are described, e.g., in Zhou and Kantardjieff (Eds.), Mammalian Cell Cultures for Biologics Manufacturing (Advances in Biochemical Engineering/Biotechnology), Springer (2014). Compositions described herein may include a vector, such as a viral vector, e.g., a lentiviral vector, encoding a recombinant protein. In some embodiments, a vector, e.g., a viral vector, comprises a nucleic acid molecule encoding a recombinant protein.

Purification of protein therapeutics is described in, e.g., Franks, Protein Biotechnology: Isolation, Characterization, and Stabilization, Humana Press (2013); and in Cutler, Protein Purification Protocols (Methods in Molecular Biology), Humana Press (2010).

Formulation of protein therapeutics is described in, e.g., Meyer (Ed.), Therapeutic Protein Drug Products: Practical Approaches to formulation in the Laboratory, Manufacturing, and the Clinic, Woodhead Publishing Series (2012).

### Modulating Gene Expression

In some embodiments, systems and methods provided herein may reversibly modulate gene expression, e.g., modifying DNA. For example, in some embodiments, transient modulation of gene expression is modulation that is time delimited, e.g., a modulation that persists for no more than about 30 mins to about 7 days, or no more than about 1 hr, 2 hrs, 3 hrs, 4 hrs, 5 hrs, 6 hrs, 7 hrs, 8 hrs, 9 hrs, 10 hrs, 11 hrs, 12 hrs, 13 hrs, 14 hrs, 15 hrs, 16 hrs, 17 hrs, 18 hrs, 19 hrs, 20 hrs, 21 hrs, 22 hrs, 24 hrs, 36 hrs, 48 hrs, 60 hrs, 72 hrs, 4 days, 5 days, 6 days, 7 days, or any time therebetween.

In some embodiments, systems or methods provided herein may irreversibly modulate gene expression, e.g., modifying DNA. For example, in some embodiments, stable modulation of gene expression is modulation that persists for a particular period of time, e.g., a modulation that persists for at least about 1 hr to about 30 days, or at least about 2 hrs, 6 hrs, 12 hrs, 18 hrs, 24 hrs, 2 days, 3, days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, or longer or any time therebetween.

In some aspects, the present disclosure provides a vector comprising a nucleic acid encoding one or more components described herein. In some embodiments, a vector, e.g., a viral vector, comprises one or more nucleic acids described herein.

In some aspects, the present disclosure provides a pharmaceutical composition comprising a cell, e.g., plurality of cells, modified to express systems described herein, e.g., one or more components.

In another aspect, the present disclosure provides a cell or tissue comprising systems provided herein, e.g., a nucleic acid encoding one or more components described herein.

In some embodiments, nucleic acids as described herein or nucleic acids encoding a component as described herein, e.g., incomplete effector moiety and/or DNA targeting moiety, may be incorporated into a vector. In some embodiments, systems provided herein comprise one or more vectors comprising one or more nucleic acid sequences encoding incomplete effector moieties as provided herein and one or more nucleic acid sequences encoding DNA targeting moieties as provided herein. In some embodiments, systems provided herein further comprises one or more vectors comprising one or more nucleic acid sequences encoding the incomplete effector moieties and one or more DNA targeting moieties. In some embodiments, vectors, including those derived from retroviruses such as lentivirus, are suitable tools to achieve long-term gene transfer, including, e.g. because they may allow long-term, stable integration of a transgene and its propagation in daughter cells. By way of non-limiting example, vectors include expression vectors, replication vectors, probe generation vectors, and/or sequencing vectors. In some embodiments, an expression vector may be provided to a cell in the form of a viral vector. As will be appreciated by one of skill in the art, certain viral vector technology is well known and described in a variety of virology and molecular biology manuals. By way of non-limiting example, viruses, which may be useful as vectors include, but are not limited to, retroviruses, adenoviruses, adeno-associated viruses, herpes viruses, and lentiviruses. In some embodiments, a suitable vector contains an origin of replication functional in at least one organism, a promoter sequence, convenient restriction endonuclease sites, and one or more selectable markers.

In some embodiments, expression of natural or synthetic nucleic acids is achieved by operably linking a nucleic acid encoding a gene of interest to a promoter, and incorporating a construct comprising the gene of interest into an expression vector. In some embodiments, vectors can be suitable for replication and integration in eukaryotes. In some such embodiments, typical cloning vectors contain transcription and translation terminators, initiation sequences, and promoters useful for expression of a desired nucleic acid sequence.

In some embodiments, additional promoter elements, e.g., enhancers, regulate the frequency of transcriptional initiation. In some such embodiments, such additional promoter elements may be located in a region 30-110 bp upstream of a known translation start site, although a number of promoters have recently been shown to contain functional elements downstream of a start site as well. In some embodiments, spacing between promoter elements frequently is flexible, e.g., so that promoter function is preserved when elements are inverted or moved relative to one another. For example, in a thymidine kinase (tk) promoter, spacing between promoter elements can be increased to 50 bp apart before promoter activity begins to decline. In some embodiments (including depending on a given promoter), it appears that individual elements can function either cooperatively or independently to activate transcription.

For example, in some embodiments, an exemplary suitable promoter is the immediate early cytomegalovirus (CMV) promoter sequence. In some embodiments, this promoter sequence is a strong constitutive promoter sequence capable of driving high levels of expression of any polynucleotide sequence operatively linked thereto. In some embodiments, an exemplary suitable promoter is Elongation Growth Factor-1α (EF-1α). In some embodiments, any constitutive promoter sequence(s) may also be used, including, but not limited to simian virus 40 (SV40) early promoter, mouse mammary tumor virus (MMTV), human immunodeficiency virus (HIV) long terminal repeat (LTR) promoter, MoMuLV promoter, avian leukemia virus promoter, Epstein-Barr virus immediate early promoter, rous sarcoma virus promoter, as well as human gene promoters such as, but not limited to, actin promoter, myosin promoter, hemoglobin promoter, and/or creatine kinase promoter.

Further, the present disclosure is not limited to use of constitutive promoters. In some embodiments, use of inducible promoters is also contemplated in technologies provided by the present disclosure. In some embodiments, use of an inducible promoter provides a molecular switch capable of turning on expression of a polynucleotide sequence to which it is operatively linked when such expression is desired, or turning off expression when such expression is not desired. For example, inducible promoters may include, but are not limited to metallothionine promoter, glucocorticoid promoter, progesterone promoter, and tetracycline promoter.

In some embodiments, an expression vector to be introduced can also contain either a selectable marker gene or a reporter gene or both to facilitate identification and selection of expressing cells from a population of cells sought to be transfected or infected through viral vectors. In other embodiments, a selectable marker may be carried on a separate piece of DNA and used in a co-transfection procedure. In some embodiments, both selectable markers and reporter genes may be flanked with appropriate regulatory sequences to enable expression in host cells. It is contemplated that, in some embodiments, useful selectable markers include, for example, antibiotic-resistance genes, such as neo and the like.

In some embodiments, reporter genes may be used for identifying potentially transfected cells and for evaluating functionality of regulatory sequences. In some embodiments, a reporter gene is a gene that is not present in or expressed by a recipient source, and that encodes a polypeptide whose expression is manifested by some easily detectable property, e.g., enzymatic activity, visualizable fluorescence, etc. Expression of such a reporter gene may be assayed at a suitable time after DNA has been introduced into recipient cells. In some embodiments, suitable reporter genes may include genes encoding luciferase, beta-galactosidase, chloramphenicol acetyl transferase, secreted alkaline phosphatase, and/or green fluorescent protein (e.g., Ui-Tei et al., 2000 FEBS Letters 479: 79-82). As will be understood by one of skill in the art, suitable expression systems are well known and may be prepared using known techniques or obtained commercially. In some embodiments, a construct with a minimal 5′ flanking region showing highest level of expression of a given reporter gene is identified as the promoter of a given gene. In some such embodiments, promoter regions may be linked to a reporter gene and used to evaluate agents for ability to modulate promoter-driven transcription.

### Methods of Use

The present disclosure provides an insight that current delivery technologies may have inadvertent effects, e.g., genome wide removal of transcription factors from DNA. Thus, it is contemplated that in some embodiments, technologies provided by the present disclosure may modulate transcription of a gene and/or chromatin topology/epigenetic changes to chromatin by delivering systems as provided herein without off-target, e.g., widespread or genome-wide, effects, e.g., removal of transcription factors. In some embodiments, delivering systems as provided herein, at doses sufficient to modulate transcription of a gene, does not significantly alter off-target transcriptional activity, e.g., an alteration of less than 50%, 40%, 20%, 15%, 10%, 5%, 4%, 3%, 2%, 1%, or any percentage therebetween of transcriptional activity of one or more off-targets as compared to activity after delivery of an effector alone.

In some embodiments, methods and systems provided herein to modify a target site may be inducible. In some embodiments, it is contemplated that use of an inducible alteration to a target site provides a molecular switch capable of turning on an alteration, or turning off an alteration when such an alteration is not desired. By way of non-limiting example, in some embodiments, systems used for inducing alterations include, but are not limited to an inducible targeting moiety based on a prokaryotic operon, e.g., lac operon, transposon Tn10, tetracycline operon, and the like, and an inducible targeting moiety based on a eukaryotic signaling pathway, e.g. steroid receptor-based expression systems, e.g. estrogen receptor or progesterone-based expression system, metallothionein-based expression system, ecdysone-based expression system, etc. In some embodiments, methods and systems provided herein include an inducible composition or components thereof comprising a DNA targeting moiety operably linked to an incomplete effector moiety.

In some embodiments, methods and systems provided herein also may modify a target site by preventing, inhibiting, and/or interfering with activity of other effector proteins at a target site. For example, in some embodiments, specific binding to a target site or adjacent to a target site by DNA targeting moieties operably linked to incomplete effector moieties may prevent an epigenetic modifying enzyme, e.g., methyltransferase, from binding to that target site or a region adjacent to that target site.

In some embodiments, methods and compositions provided herein treat disease by stably or transiently modifying a target site to alter gene expression. In some such embodiments, a target site is altered to result in a stable modulation of gene expression, such as a modulation that persists for at least about 1 hr to about 30 days, or at least about 2 hrs, 6 hrs, 12 hrs, 18 hrs, 24 hrs, 2 days, 3, days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, or longer or any time therebetween. In some embodiments, a target site is altered to result in a transient modification of a target site to modulate gene expression, such as a modulation that persists for no more than about 30 mins to about 7 days, or no more than about 1 hr, 2 hrs, 3 hrs, 4 hrs, 5 hrs, 6 hrs, 7 hrs, 8 hrs, 9 hrs, 10 hrs, 11 hrs, 12 hrs, 13 hrs, 14 hrs, 15 hrs, 16 hrs, 17 hrs, 18 hrs, 19 hrs, 20 hrs, 21 hrs, 22 hrs, 24 hrs, 36 hrs, 48 hrs, 60 hrs, 72 hrs, 4 days, 5 days, 6 days, 7 days, or any time therebetween.

In some aspects, the present disclosure provides methods of modifying a target site comprising binding a first component comprising a first incomplete effector moiety with a nucleic acid sequence adjacent to the target site and binding a second component comprising a second incomplete effector moiety with a different nucleic acid sequence that is also adjacent to the target site, wherein effector activity is induced at the target site. It is contemplated that in some such embodiments, binding both components to the nucleic acid sequences adjacent to the target site allows interaction between the first and second components which induces the effector activity at the target site.

In some embodiments, effector activity is selected from the group consisting of DNA methyltransferase, histone methyltransferase, deaminase, acetyltransferase, histone deacetylase, ligase, nuclease, phosphatase, recombinase, transposase, and polynucleotide kinase activity. In some embodiments, effector activity at a target site modulates gene expression. In some embodiments, effector activity at a target site modulates chromatin topology and/or induces epigenetic changes to chromatin. In some embodiments, chromatin and/or epigenetic changes modulate gene expression. In some embodiments, interaction of two incomplete effector moieties is sufficient to provide an effector activity at or near the target site is, e.g., an increase of effector activity at least 10%, 20%, 30%, 40%, 50%, 60%, 70%, 80%, 85%, 90%, 95%, or any percentage therebetween as compared to effector activity of either of the incomplete effector moieties alone.

In some embodiments, it is contemplated that systems and methods provided herein are useful to modify a target site in a plant. In some embodiments, such methods comprise modifying gene expression or chromatin topology in plants and/or crops for altering properties in the plants and/or crops, e.g., increasing drought tolerance, pathogen resistance, herbicide/toxin resistance, metabolic engineering, yield, and/or nutritional value. For a list of example plant genes associated with disease resistance, see, e.g., Hammond-Kosack et al., Ann. Rev. Plant Physiol. Plant Mol. Biol., 1997, 48:575-607; Table 1 from Sekhwal et al., Int. J. Mol. Sci., 2015, 16:19248-19290. See also, Kromdijk et al., Science, 2016, 354:857-861, for improving crop productivity.

### Formulation and Administration

In some embodiments, pharmaceutical compositions provided herein may be formulated for delivery via any route of administration. In some embodiments, modes of administration include injection, infusion, instillation, or ingestion. Injection includes, without limitation, intravenous, intramuscular, intra-arterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and/or infusion. In some embodiments, administration includes aerosol inhalation, e.g., with nebulization. In some embodiments, administration is systemic (e.g., oral, rectal, nasal, sublingual, buccal, or parenteral), enteral (e.g., system-wide effect, but delivered through the gastrointestinal tract), and/or local (e.g., local application on the skin, intravitreal injection). In some embodiments, a composition is administered systemically. In some embodiments, administration is non-parenteral and a provided therapeutic is a parenteral therapeutic.

In some embodiments, the present disclosure provides pharmaceutical compositions described herein comprising a pharmaceutically acceptable excipient. In some embodiments, pharmaceutically acceptable excipients include an excipient that is useful in preparing a pharmaceutical composition that is generally safe, non-toxic, and desirable, such as, e.g., excipients that are acceptable for veterinary use as well as for human pharmaceutical use. For example, in some embodiments, excipients may be solid, liquid, semisolid, or, in the case of an aerosol composition, gaseous.

In some embodiments, pharmaceutical compositions provided herein may also be tableted or prepared in an emulsion or syrup for oral administration. In some embodiments, pharmaceutically acceptable solid or liquid carriers may be added to enhance or stabilize a composition, or to facilitate preparation of a composition. In some embodiments, liquid carriers include syrup, peanut oil, olive oil, glycerin, saline, alcohols and/or water. In some embodiments, solid carriers include starch, lactose, calcium sulfate, dihydrate, terra alba, magnesium stearate or stearic acid, talc, pectin, acacia, agar and/or gelatin. In some embodiments, a carrier may also include a sustained release material such as, e.g., glyceryl monostearate or glyceryl distearate, alone or with a wax.

In some embodiments, pharmaceutical preparations are made following conventional techniques of pharmacy, as will be known to those of skill in the art, such as, e.g. those involving milling, mixing, granulation, and/or compressing, when necessary, for tablet forms; or milling, mixing and/or filling for hard gelatin capsule forms. In some embodiments, when a liquid carrier is used, a preparation will be in the form of a syrup, elixir, emulsion or an aqueous or non-aqueous suspension. In some such embodiments, a liquid formulation may be administered directly per os.

In some embodiments, pharmaceutical compositions according to the present disclosure may be delivered in a therapeutically effective amount. In some embodiments, a precise therapeutically effective amount is that amount of a composition that will yield most effective results in terms of efficacy of treatment in a given subject. In some such embodiments, this amount will vary depending upon a variety of factors, including but not limited to, e.g., characteristics of a provided therapeutic compound (including activity, pharmacokinetics, pharmacodynamics, and bioavailability), physiological condition of a subject (including age, sex, disease type and stage, general physical condition, responsiveness to a given dosage, and type of medication), nature of a given pharmaceutically acceptable carrier or carriers in a provided formulation, and route of administration. One skilled in clinical and pharmacological arts will be able to determine a therapeutically effective amount through routine experimentation, for instance, by monitoring a subject's response to administration of a compound and adjusting dosage accordingly. For additional guidance, see Remington: The Science and Practice of Pharmacy (Gennaro ed. 22.sup.nd edition, Williams & Wilkins PA, USA) (2012).

In some embodiments, pharmaceutical compositions described herein may be formulated for example including a carrier, such as a pharmaceutical carrier and/or a polymeric carrier, e.g., a liposome, and delivered by known methods to a subject in need thereof (e.g., a human or non-human agricultural or domestic animal, e.g., cattle, dog, cat, horse, poultry). In some embodiments, such methods may include, e.g. transfection (e.g., lipid-mediated, cationic polymers, calcium phosphate); electroporation (e.g., nucleofection) and/or viral delivery (e.g., lentivirus, retrovirus, adenovirus, AAV). Certain methods of delivery are also described, e.g., in Gori et al., Delivery and Specificity of CRISPR/Cas9 Genome Editing Technologies for Human Gene Therapy. Human Gene Therapy. July 2015, 26(7): 443-451. doi:10.1089/hum.2015.074; and Zuris et al. Cationic lipid-mediated delivery of proteins enables efficient protein-based genome editing in vitro and in vivo. Nat Biotechnol. 2014 Oct. 30; 33(1):73-80.

In some embodiments, liposomes are spherical vesicle structures composed of a uni- or multilamellar lipid bilayer surrounding internal aqueous compartments and a relatively impermeable outer lipophilic phospholipid bilayer. In some embodiments, liposomes may be anionic, neutral or cationic. In some embodiments, liposomes are biocompatible, nontoxic, can deliver both hydrophilic and lipophilic drug molecules, protect their cargo from degradation by plasma enzymes, and/or transport their load across biological membranes and the blood brain barrier (BBB) (see, e.g., Spuch and Navarro, Journal of Drug Delivery, vol. 2011, Article ID 469679, 12 pages, 2011. doi:10.1155/2011/469679 for review).

In some embodiments, vesicles can be made from several different types of lipids; however, phospholipids are most commonly used to generate liposomes as drug carriers. In some embodiments, vesicles may comprise, without limitation, DOTMA, DOTAP, DOTIM, DDAB, alone or together with cholesterol to yield DOTMA and cholesterol, DOTAP and cholesterol, DOTIM and cholesterol, and DDAB and cholesterol. Methods for preparation of multilamellar vesicle lipids are known in the art (see for example U.S. Pat. No. 6,693,086, the teachings of which relating to multilamellar vesicle lipid preparation are incorporated herein by reference). In some embodiments, although vesicle formation maybe spontaneous when a lipid film is mixed with an aqueous solution, in some embodiments, vesicle formation may also be expedited by applying force by shaking using a homogenizer, sonicator, and/or an extrusion apparatus (see, e.g., Spuch and Navarro, Journal of Drug Delivery, vol. 2011, Article ID 469679, 12 pages, 2011. doi:10.1155/2011/469679 for review). In some embodiments, extruded lipids can be prepared by extruding through filters of decreasing size, as described in Templeton et al., Nature Biotech, 15:647-652, 1997, the teachings of which relating to extruded lipid preparation are incorporated herein by reference.

In some embodiments as described herein, additives may be added to vesicles to modify their structure and/or properties. For example, in some embodiments, either cholesterol or sphingomyelin may be added to a mixture in order to help stabilize structure and to prevent leakage of inner cargo. In some embodiments, vesicles can be prepared from hydrogenated egg phosphatidylcholine or egg phosphatidylcholine, cholesterol, and dicetyl phosphate. (see, e.g., Spuch and Navarro, Journal of Drug Delivery, vol. 2011, Article ID 469679, 12 pages, 2011. doi:10.1155/2011/469679 for review). In some embodiments, vesicles may be surface modified during or after synthesis to include reactive groups complementary to reactive groups on carrier cells. In some such embodiments, reactive groups include without limitation maleimide groups. For example, in some embodiments, vesicles may be synthesized to include maleimide conjugated phospholipids such as, e.g., DSPE-MaL-PEG2000.

In some embodiments, vesicle formulation may be mainly comprised of natural phospholipids and lipids such as 1,2-distearoryl-sn-glycero-3-phosphatidyl choline (DSPC), sphingomyelin, egg phosphatidylcholines and monosialoganglioside. In some embodiments, formulations made up of phospholipids only are less stable in plasma. In some embodiments, however, manipulation of a lipid membrane with cholesterol reduces rapid release of an encapsulated bioactive compound into cellular plasma or 1,2-dioleoyl-sn-glycero-3-phosphoethanolamine (DOPE) increases stability (see, e.g., Spuch and Navarro, Journal of Drug Delivery, vol. 2011, Article ID 469679, 12 pages, 2011. doi:10.1155/2011/469679 for review).

In some embodiments, lipids may be used to form lipid microparticles. For example, in some embodiments, lipids include, but are not limited to, DLin-KC2-DMA4, C12-200 and colipids disteroylphosphatidyl choline, cholesterol, and PEG-DMG may be formulated (see, e.g., Novobrantseva, Molecular Therapy-Nucleic Acids (2012) 1, e4; doi:10.1038/mtna.2011.3) using a spontaneous vesicle formation procedure. In some embodiments, a component molar ratio may be about 50/10/38.5/1.5 (DLin-KC2-DMA or C12-200/disteroylphosphatidyl choline/cholesterol/PEG-DMG). Tekmira has a portfolio of approximately 95 patent families, in the U.S. and abroad, that are directed to various aspects of lipid microparticles and lipid microparticles formulations (see, e.g., U.S. Pat. Nos. 7,982,027; 7,799,565; 8,058,069; 8,283,333; 7,901,708; 7,745,651; 7,803,397; 8,101,741; 8,188,263; 7,915,399; 8,236,943 and 7,838,658 and European Pat. Nos. 1766035; 1519714; 1781593 and 1664316), all of which may be used and/or adapted to the present disclosure.

In some embodiments, at least one composition of systems provided herein further comprises a nanoparticle, liposome, and/or exosome.

In some embodiments, methods and compositions provided herein may comprise a pharmaceutical composition administered by a regimen sufficient to alleviate a symptom of a disease, disorder and/or condition. In some aspects, the present disclosure provides methods of delivering a therapeutic by administering a composition described herein.

In some embodiments, pharmaceutical compositions are also described that include any \compositions as described herein. In some aspects, the present disclosure provides compositions formulated as pharmaceutical compositions. In another aspect, the present disclosure provides a pharmaceutical composition comprising a cell modified to express systems provided herein. In some such embodiments, systems provided herein are effective to provide an effector activity at or near a target site, in at least a human cell.

### Methods of Treatment

Systems and methods provided herein can be used to treat disease in human and non-human animals. In some aspects, the present disclosure provides methods of treating a disease or condition (e.g., sufficient to treat or reduce a symptom by at least 5%, 10%, 15%, 20%, 25%, 30%, 35%, 40%, 45%, 50%, 55%, 60%, 65% 70%, 75%, 80%, 85%, 90%, 95% or greater) comprising administering systems provided herein.

For example, in some embodiments, oncology indications can be targeted by use of technologies of the present disclosure to repress oncogenes and/or activate tumor suppressors. In some embodiments, diseases characterized by nucleotide repeats, e.g., trinucleotide repeats in which silencing of the gene through methylation drives symptoms, can be targeted by use of technologies of the present disclosure to modify gene expression. In some such embodiments, examples of such diseases include: DRPLA (Dentatorubropallidoluysian atrophy), HD (Huntington's disease), SBMA (Spinal and bulbar muscular atrophy), SCA1 (Spinocerebellar ataxia Type 1), SCA2 (Spinocerebellar ataxia Type 2), SCA3 (Spinocerebellar ataxia Type 3 or Machado-Joseph disease), SCA6 (Spinocerebellar ataxia Type 6), SCA7 (Spinocerebellar ataxia Type 7), SCA17 (Spinocerebellar ataxia Type 17), FRAXA (Fragile X syndrome), FXTAS (Fragile X-associated tremor/ataxia syndrome), FRAXE (Fragile XE mental retardation), FRDA (Friedreich's ataxia) FXN or X25, DM (Myotonic dystrophy), SCA8 (Spinocerebellar ataxia Type 8) and SCA12 (Spinocerebellar ataxia Type 12). In addition, diseases characterized by an overexpressed/dominant negative gene, such as an oncogene driven cancer (e.g., MYC addicted cancers, Bcr-Abl), severe congenital neutropenia, and Huntington's chorea, may be targeted by technologies of the present disclosure.

In some embodiments, expression of a gene is modulated, e.g., transcription of a target nucleic acid sequence, as compared with a reference value, e.g., transcription of a target sequence in absence of interaction between incomplete effector moieties (e.g., sufficient to modulate expression by at least 5%, 10%, 15%, 20%, 25%, 30%, 35%, 40%, 45%, 50%, 55%, 60%, 65% 70%, 75%, 80%, 85%, 90%, 95% or greater).

Systems and methods provided herein may be used to treat severe congenital neutropenia (SCN). In some embodiments, expression of the ELANE gene (e.g., sufficient to decrease or inhibit expression by at least 5%, 10%, 15%, 20%, 25%, 30%, 35%, 40%, 45%, 50%, 55%, 60%, 65% 70%, 75%, 80%, 85%, 90%, 95% or greater), which causes the disease, is inhibited. In some embodiments, a system comprising a first nucleic acid sequence encoding a first incomplete effector moiety, a first DNA targeting moiety that interacts with the first incomplete effector moiety and binds to a first target DNA site, a second nucleic acid sequence encoding a second incomplete effector moiety, and a second DNA targeting moiety that interacts with the second incomplete effector moiety and binds to a second target DNA site adjacent to the first target site is administered to target one or more target DNA sites adjacent to the ELANE gene to repress (e.g. by alteration) the ELANE gene. In some embodiments, systems comprising a first composition comprising a first component comprising a first DNA targeting moiety which binds to a first target DNA site, operably linked to a first incomplete effector moiety and second composition comprising a second component comprising a second DNA targeting moiety which binds to a second target DNA site adjacent to the first target site, operably linked to a second incomplete effector moiety is administered. In some embodiments, first and second components may interact to provide an effector activity at or near a target site to target one or more target DNA sites adjacent to the ELANE gene to repress (e.g. by alteration) the ELANE gene.

In some aspects, the present disclosure provides a method of treating SCN with a pharmaceutical composition described herein. In some embodiments, administration of systems provided herein modulates gene expression of one or more genes, such as by inhibiting gene expression of the ELANE gene, to treat SCN (e.g., sufficient to decrease or inhibit expression by at least 5%, 10%, 15%, 20%, 25%, 30%, 35%, 40%, 45%, 50%, 55%, 60%, 65% 70%, 75%, 80%, 85%, 90%, 95% or greater).

In some embodiments, systems and methods provided herein may be used to treat sickle cell anemia and beta thalassemia. In some embodiments, expression of HbF from the HBG genes, shown to restore normal hemoglobin levels, is activated. In some embodiments, a system provided herein is administered to target one or more sequences adjacent in the HBB gene cluster and/or the HBG genes. In some embodiments, the HBB gene cluster is inhibited. In some embodiments, one or more of the HBG genes is activated.

In some aspects, the present disclosure provides a method of treating sickle cell anemia and beta thalassemia with a pharmaceutical composition provided herein. In some embodiments, administration of a system provided herein modulates gene expression of one or more genes, such as modulating gene expression from the HBB gene cluster or the HBG genes, to treat SCN.

In some embodiments, systems and methods provided herein may be used to treat MYC-related tumors. In some embodiments, expression of MYC (which has been shown to cause tumors) is inhibited. In some embodiments, a system provided herein is administered to target one or more sequences in or adjacent to the MYC gene. In some embodiments, the MYC gene is inhibited (e.g., sufficient to decrease or inhibit expression by at least 5%, 10%, 15%, 20%, 25%, 30%, 35%, 40%, 45%, 50%, 55%, 60%, 65% 70%, 75%, 80%, 85%, 90%, 95% or greater).

In some aspects, the present disclosure provides a method of treating MYC-related tumors with a pharmaceutical composition provided herein. In some embodiments, administration of a system provided herein modulates gene expression of one or more genes, such as, e.g. modulating gene expression from the MYC gene, to treat MYC-related tumors.

In some embodiments, compositions and methods described herein may be used to treat myoclonic epilepsy of infancy (SMEI or Dravet's syndrome). In some embodiments, loss-of-function mutations in Na 1.1, also known as the sodium channel, voltage-gated, type I, alpha subunit (SCN1A), from the SCN1A gene, cause severe Dravet's syndrome. In some embodiments, a system provided herein is administered to target one or more sequences in or adjacent to the SCN1A gene. In some embodiments, a system provided herein is administered to target one or more sequences adjacent in the SCN3A gene to increase expression of Naᵥ1.3, also known as the sodium channel, voltage-gated, type III, alpha subunit (SCN3A). In some embodiments, a system provided herein is administered to target one or more sequences in or adjacent to the SCN5A gene, to increase expression of Naᵥ1.5, also known as the sodium channel, voltage-gated, type V, alpha subunit (SCN5A). In some embodiments, a system provided herein is administered to target one or more sequences in or adjacent to the SCN8A gene to increase expression of Naᵥ1.6, also known as the sodium channel, voltage-gated, type VIII, alpha subunit (SCN8A). In some embodiments, any one of SCN1A, SCN3A, SCN5A, and SCN8A genes is activated to increase expression of Naᵥ1.1, Naᵥ1.3, Naᵥ1.5, and Naᵥ1.6, respectively (e.g., sufficient to activate or increase expression by at least 5%, 10%, 15%, 20%, 25%, 30%, 35%, 40%, 45%, 50%, 55%, 60%, 65% 70%, 75%, 80%, 85%, 90%, 95% or greater).

In some aspects, the present disclosure provides a method of treating Dravet's syndrome with a pharmaceutical composition described herein. In one embodiment, administration of a system described herein modulates gene expression of one or more genes, such as modulating gene expression from the SCN1A, SCN3A, SCN5A, and SCN8A genes, to treat Dravet's syndrome.

In some embodiments, compositions and methods described herein may be used to treat familial erythromelalgia. In some embodiments, loss-of-function mutations in Naᵥ1.7, also known as the sodium channel, voltage-gated, type IX, alpha subunit (SCN9A), from the SCN9A gene, cause severe familial erythromelalgia. In some embodiments, a system provided herein is administered to target one or more sequences in or adjacent to the SCN9A gene. In one embodiment, the SCN9A gene is activated to increase expression of Naᵥ1.7.

In some aspects, the present disclosure provides a method of treating familial erythromelalgia with a pharmaceutical composition provided herein. In some embodiments, administration of a system described herein modulates gene expression of one or more genes, such as modulating gene expression from the SCN9A gene, to treat familial erythromelalgia.

Cancer Therapies

In some embodiments, compositions and methods described herein may be used to treat cancer. In some embodiments, cancer or neoplasm includes solid or liquid cancer and includes benign and/or malignant tumors, and/or hyperplasias, including, e.g., gastrointestinal cancer (such as non-metastatic or metastatic colorectal cancer, pancreatic cancer, gastric cancer, esophageal cancer, hepatocellular cancer, cholangiocellular cancer, oral cancer, lip cancer); urogenital cancer (such as hormone sensitive or hormone refractory prostate cancer, renal cell cancer, bladder cancer, penile cancer); gynecological cancer (such as ovarian cancer, cervical cancer, endometrial cancer); lung cancer (such as small-cell lung cancer and non-small-cell lung cancer); head and neck cancer (e.g. head and neck squamous cell cancer); CNS cancer including malignant glioma, astrocytomas, retinoblastomas and brain metastases; malignant mesothelioma; non-metastatic or metastatic breast cancer (e.g. hormone refractory metastatic breast cancer); skin cancer (such as malignant melanoma, basal and squamous cell skin cancers, Merkel Cell Carcinoma, lymphoma of the skin, Kaposi Sarcoma); thyroid cancer; bone and soft tissue sarcoma; and hematologic neoplasias (such as multiple myeloma, acute myelogenous leukemia, chronic myelogenous leukemia, myelodysplastic syndrome, acute lymphoblastic leukemia, Hodgkin's lymphoma).

In some aspects, the present disclosure provides a method of treating a cancer with a pharmaceutical composition provided herein. In some embodiments, administration of a system described herein modulates gene expression of one or more genes, such as inhibiting gene expression of an oncogene, to treat a cancer.

For example, in some embodiments, oncology indications can be targeted by use of the present disclosure to repress oncogenes (e.g., MYC, RAS, HER1, HER2, JUN, FOS, SRC, RAF, etc.) and/or activate tumor suppressors (e.g., P16, P53, P73, PTEN, RB1, BRCA1, BRCA2, etc.) (e.g., sufficient to modulate expression by at least 5%, 10%, 15%, 20%, 25%, 30%, 35%, 40%, 45%, 50%, 55%, 60%, 65% 70%, 75%, 80%, 85%, 90%, 95% or greater).

Neurological Diseases or Disorder

In some embodiments, methods provided herein may also treat a neurological disease. A “neurological disease” or “neurological disorder” as used herein, is a disease or disorder that affects the nervous system of a subject including a disease that affects the brain, spinal cord, or peripheral nerves. A neurological disease or disorder may affect nerve cells (e.g. neurons and precursors thereof) or the supporting cells of the nervous system (e.g. glial cells, e.g. astrocytes, oligodendrocytes, microglia, etc., and precursors thereof). In some embodiments, causes of neurological disease or disorder include infection, inflammation, ischemia, injury, tumor, or inherited illness. In some embodiments, neurological diseases or disorders also include neurodegenerative diseases and myodegenerative diseases. For example, in some embodiments, neurodegenerative diseases include, but are not limited to, amyotrophic lateral sclerosis, Alzheimer's disease, frontotemporal dementia, frontotemporal dementia with TDP-43, frontotemporal dementia linked to chromosome-17, Pick's disease, Parkinson's disease, Huntington's disease, Huntington's chorea, mild cognitive impairment, Lewy Body disease, multiple system atrophy, progressive supranuclear palsy, an α-synucleinopathy, a tauopathy, a pathology associated with intracellular accumulation of TDP-43, and cortico-basal degeneration in a subject. In some embodiments, examples of neurological diseases or disorders include, but are not limited to, tinnitus, epilepsy, depression, stroke, multiple sclerosis, migraines, and anxiety.

In some aspects, the present disclosure provides a method of treating a neurological disease or disorder with a pharmaceutical composition provided herein. In some embodiments, administration of a system described herein modulates activation of a neurotransmitter, neuropeptide, or neuroreceptor (e.g., sufficient to modulate expression by at least 5%, 10%, 15%, 20%, 25%, 30%, 35%, 40%, 45%, 50%, 55%, 60%, 65% 70%, 75%, 80%, 85%, 90%, 95% or greater).

In some embodiments, for example, systems of the present disclosure can be used to modulate neuroreceptor activity (e.g., adrenergic receptor, GABA receptor, acetylcholine receptor, dopamine receptor, serotonin receptor, cannabinoid receptor, cholecystokinin receptor, oxytocin receptor, vasopressin receptor, corticotropin receptor, secretin receptor, somatostatin receptor, etc.) by activating expression of a neurotransmitter, neuropeptide, agonist or antagonist thereof (e.g., acetylcholine, dopamine, norepinephrine, epinephrine, serotonin, melatonin, cirodhamine, oxytocin, vasopressin, cholecystokinin, neurophysins, neuropeptide Y, enkephalin, orexins, somatostatin, etc.).

Treatments for Acute and Chronic Infections

In some embodiments, methods provided herein may also improve existing acute and chronic infection therapeutics to increase bioavailability and reduce toxicokinetics. As used herein, “acute infection” refers to an infection that is characterized by a rapid onset of disease or symptoms. As used herein, by “persistent infection” or “chronic infection” is meant an infection in which the infectious agent (e.g., virus, bacterium, parasite, mycoplasm, or fungus) is not cleared or eliminated from the infected host, even after the induction of an immune response. In some embodiments, persistent infections may be chronic infections, latent infections, or slow infections. In some embodiments, acute infections are relatively brief (lasting a few days to a few weeks) and resolved from a body of an organism by its immune system. In some embodiments, persistent infections may last for months, years, or even a lifetime. In some such embodiments, infections may also recur frequently over a long period of time, involving stages of silent and productive infection without cell killing or even producing excessive damage to host cells. In some embodiments, mammals are diagnosed as having a persistent infection according to any standard method known in the art and described, for example, in U.S. Pat. Nos. 6,368,832, 6,579,854, and 6,808,710.

In some embodiments, infection is caused by one or more pathogens from one of the following major categories:

i) viruses, including members of the Retroviridae family such as the lentiviruses (e.g. Human immunodeficiency virus (HIV) and deltaretroviruses (e.g., human T cell leukemia virus I (HTLV-I), human T cell leukemia virus II (HTLV-II)); Hepadnaviridae family (e.g. hepatitis B virus (HBV)), Flaviviridae family (e.g. hepatitis C virus (HCV)), Adenoviridae family (e.g. Human Adenovirus), Herpesviridae family (e.g. Human cytomegalovirus (HCMV), Epstein-Barr virus, herpes simplex virus 1 (HSV-1), herpes simplex virus 2 (HSV-2), human herpesvirus 6 (HHV-6), varicella-zoster virus), Papillomaviridae family (e.g. Human Papillomavirus (HPV)), Parvoviridae family (e.g. Parvovirus B19), Polyomaviridae family (e.g. JC virus and BK virus), Paramyxoviridae family (e.g. Measles virus), Togaviridae family (e.g. Rubella virus) as well as other viruses such as hepatitis D virus;

ii) bacteria, such as those from the following families: Salmonella (e.g. S. enterica Typhi), Mycobacterium (e.g. M. tuberculosis and M. leprae), Yersinia (Y. pestis), Neisseria (e.g. N. meningitides, N. gonorrhea), Burkholderia (e.g. B. pseudomallei), Brucella, Chlamydia, Helicobacter, Treponema, Borrelia, Rickettsia, and Pseudomonas;

iii) parasites, such as Leishmania, Toxoplasma, Trypanosoma, Plasmodium, Schistosoma, or Encephalitozoon; and

iv) prions, such as prion protein.

In some embodiments, administration of compositions provided herein suppresses transcription or activates transcription of one or more genes to treat an infection such as a viral infection. In some embodiments, for example, a system provided herein may inhibit viral DNA transcription, e.g., targeting a viral gene, to treat a viral infection (e.g., sufficient to decrease inhibit viral DNA transcription by at least 5%, 10%, 15%, 20%, 25%, 30%, 35%, 40%, 45%, 50%, 55%, 60%, 65% 70%, 75%, 80%, 85%, 90%, 95% or greater).

Treatments of Other Diseases Disorders Conditions

In some embodiments, additional diseases that may be treated by compositions provided herein include, but are not limited to, imprinted or hemizygous mono-allelic diseases, bi-allelic diseases, autosomal recessive disorders, autosomal dominant disorders, and diseases characterized by nucleotide repeats, e.g., trinucleotide repeats in which silencing of a gene through methylation drives symptoms, can be targeted by use of technologies of the present disclosure to modulate expression of an affected gene. In some embodiments, for example, such diseases may include: Jacobsen syndrome, cystic fibrosis, sickle cell anemia, and Tay Sachs disease, tuberous sclerosis, Marfan syndrome, neurofibromatosis, retinoblastoma, Waardenburg syndrome, familial hypercholesterolemia, DRPLA (Dentatorubropallidoluysian atrophy), HD (Huntington's disease), Beckwith-Wiedemann syndrome, Silver-Russell syndrome, SBMA (Spinal and bulbar muscular atrophy), SCA1 (Spinocerebellar ataxia Type 1), SCA2 (Spinocerebellar ataxia Type 2), SCA3 (Spinocerebellar ataxia Type 3 or Machado-Joseph disease), SCA6 (Spinocerebellar ataxia Type 6), SCA7 (Spinocerebellar ataxia Type 7), SCA17 (Spinocerebellar ataxia Type 17), FRAXA (Fragile X syndrome), FXTAS (Fragile X-associated tremor/ataxia syndrome), FRAXE (Fragile XE mental retardation), FRDA (Friedreich's ataxia) FXN or X25, DM (Myotonic dystrophy), SCA8 (Spinocerebellar ataxia Type 8), and SCA12 (Spinocerebellar ataxia Type 12).

In some aspects, the present disclosure provides a method of treating a genetic disease/disorder/condition with a pharmaceutical composition provided herein. In some embodiments, administration of systems provided herein modulates gene expression of one or more genes that are indicated in a particular genetic disease/disorder/condition, such as activating, suppressing, or modulating expression of a gene associated with the particular genetic disease/disorder/condition.

In some aspects, the present disclosure provides a method of treating a disease/disorder/condition with a pharmaceutical composition provided herein. In some embodiments, administration of a system provided herein modulates gene expression of one or more genes to treat a particular disease/disorder/condition, such as activating, suppressing, or modulating expression of a gene associated with the particular genetic disease/disorder/condition.

All references and publications cited herein are hereby incorporated by reference.

### EXAMPLES

The following examples are provided to further illustrate some embodiments of the present disclosure, but are not intended to limit the scope of the present disclosure; it will be understood by their exemplary nature that other procedures, methodologies, or techniques known to those skilled in the art may alternatively be used.

### Example #1: Designing Methyltransferase Fragments

In eukaryotes, DNA methylation has been implicated in control of cellular processes, including differentiation, gene regulation, and embryonic development. Methylation of CpG sites in promoter sequences can lead to suppression of gene expression for conditions marked by undesired gene expression or overexpression.

Three-dimensional X-ray structures of two prokaryotic methyltransferases (MTases), M.HhaI, and M.HaeIII, reveal that both fold into two domains: a large domain encompassing most of the conserved motifs (between M.HhaI and M.HaeIII); and a small domain with a variable region and a conserved motif (different from conserved motifs in the large domain). These two domains form a cleft where a DNA substrate fits and most specific DNA-protein interactions occur at a major groove interface with the small domain.

### Construction of M.HSssI Fragments

Using sequence homology to M.HhaI (FIG. 2), fragments of M.HSssI (Uniprot: P15840) are engineered to be catalytically inactive on their own, but capable of generating a catalytically active enzyme upon binding to each other. The following two fragments are used in this example: an N-terminal fragment having residues 1-304, and a C-terminal fragment having residues 241-386. When assembled into a catalytically active enzyme, these N- and C-terminal fragments are modeled in three dimensions as in the representation shown in FIG. 3.

### Construction of DNMT3A Fragments

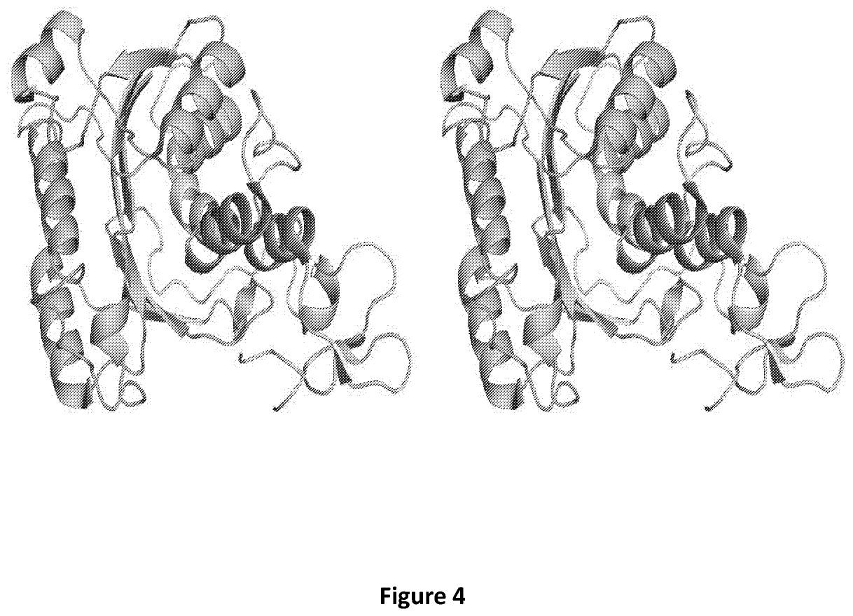

Using sequence homology to M.HhaI and M.HSssI (FIG. 2), the catalytic domain of DNMT3A (residues 634-912, derived from the plasmid pCDNA3-hDNMT3A (Addgene: 35521) or Uniprot: Q9Y6K1) is split into two fragments, designed such that each fragment is catalytically inactive on its own, but upon binding to each other a functional catalytic complex is generated. The following two fragments are engineered: an N-terminal fragment having residues 634-799, and a C-terminal fragment having residues 800-912 (FIG. 4).

### Example #2: Using a Targeted DNA to Methylate CpG Sites within PCSK9 Promoter Region to Silence PCSK9 Gene Expression

This example describes a composition selected to target specific CpG-rich regions in a PCSK9 promoter. The prokaryotic C5-MTase, M.HSssI, and/or the eukaryotic C5-MTase, DNMT3A (described in Example 1), is/are split into two fragments (e.g. an N-terminal fragment and a C-terminal fragment), each fragment of which is, in turn, joined in vitro to a single ssDNA strand that targets a region within the PCSK9 promoter. Each of the enzyme fragments are catalytically inactive (or effectively inactive) on its own, but upon binding to each other a catalytically active enzyme is generated. The ssDNA sequences pair with a promoter region of the PCSK9 gene (e.g. ssDNA sequences provide targeting mechanism), thereby directing the tethered MTase fragments to that particular genomic location (i.e. promoter region of PCSK9). When bound to their respective target regions in the PCSK9 promoter, the guiding ssDNA strands serve as a tether that allows (i) interaction between the two fragments (e.g. two fragments of DNMT3A and/or two fragments of M.HSssI) and (ii) the formation of a catalytically active MTase. The guiding ssDNA strands confer targeting specificity that further restricts the catalytically active MTase to nearby CpG sites.

Designing Guiding ssDNA Strands

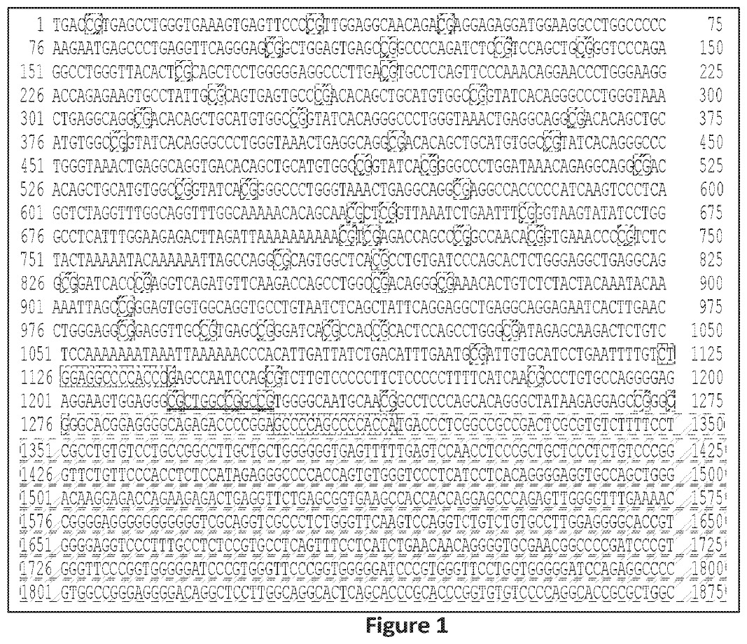

The PCSK9 promoter directly precedes the coding sequence of PCSK9 and is found between the −1 and −2000 nucleotide positions with respect to the starting ATG codon. As shown in FIG. 1 the ˜1 kb upstream region of the 5′ UTR (highlighted in grey);CpG sites are highlighted in green; the target CpG-rich area is underlined; DNA sequences targeted by guiding ssDNA strands are highlighted in teal; and upstream guiding ssDNA strand has the sequence 5′ TAACGTTTATGTTAA 3′, and the downstream guiding ssDNA strand has the sequence 5′ GACCTCACTCCAGAA 3′.

Targets for the guiding ssDNA strands are chosen with the following considerations: (a) there must be at least two targets: (a1) at least one target must be upstream (5′ direction) of the target CpG-rich area; (a2) at least one other target must be downstream (3′ direction) of the target CpG-rich area, (b) an optimal distance between the at least two targets so that, when tethered, the reconstituted catalytically active MTase (i.e. comprising the at least two fragments) is not sterically prohibited from reaching the target CpG-rich area, (c) the target CpG-rich area is localized approximately halfway between the two targets, and (d) the targets are of sufficient length to allow specificity of targeting.

### Construction of Enzyme Fragment-ssDNA Fusions

Conjugation of guiding ssDNA strands to each of the N- and C-terminal fragments of M.HSssI or DNMT3A catalytic domains described in Example 1 is performed using copper catalyzed click chemistry and an Oligo-click Kit (BaseClick). Reactions are performed as described in the manufacturer's instructions/manual, using a vial of catalyst beads, activator solution, the catalytic domain of M.HSssI and/or DNMT3A, and guiding ssDNA(s). Reactions are incubated in a Thermoshaker. Successful conjugation is confirmed by mass spectrometry.

### Cell Culture and Reporter Gene Assays

Human PCSK9 promoter (1801 bp) is amplified from HEK293T genomic DNA and cloned into a pGL3-basic luciferase reporter gene vector (Promega). HEK293T cells are cultured in DMEM (PAA laboratories GmbH), supplemented with 10% fetal calf serum (FCS) (PAA laboratories GmbH). Briefly, the HEK293T cells are seeded in 24-well plates coated with polylysine. Plasmids for co-transfection, which include a GFP reporter gene, a Renilla luciferase reporter gene controlled by cytomegalovirus (CMV) (Promega), and a target firefly luciferase reporter gene, are diluted with serum free DMEM culture medium and mixed with Transfast™ reagent (Promega). Transfection (using Transfast reagent) is performed in accordance with manufacturer's instructions. Efficiency of transfection is monitored by counting number of green fluorescent protein-positive (GFP+) cells under a fluorescence microscope (e.g. determining percentage of total number cells that are GFP+).

N- and C-terminal fragments of M.HSssI or DNMT3A, each conjugated to their respective guiding ssDNA strands, are then delivered to cells. Four days after delivery, the culture medium is removed and cells are lysed by adding lysis buffer from Renilla Luciferase Assay System (Promega, Cat. #E2810) to each well. Samples of crude cell lysate are transferred to different wells of a non-transparent micro well plate (Packard) for firefly and Renilla luciferase activity assay, respectively. Luciferase activity (luminescence signal) is determined by Topcount®NXT™ Microplate Scintillation & Luminescence Counter (Packard). In all transfection experiments, transfection yield and cell number are normalized by co-transfection with a construct expressing Renilla luciferase under the control of a CMV promoter. Luciferase activity, normalized by activity of the Renilla luciferase (not under control of PCSK9 promoter), is used as a read-out of promoter silencing.

### Bisulfite Analysis of PCKS9 Promoter Methylation

To analyze targeted DNA methylation, transfected HEK293T cells are harvested and washed with PBS. Episomal DNA is isolated using a Qiagen miniprep kit, following manufacturer protocol. Cells are harvested and total cellular DNA is isolated using DNeasy Tissue Kit (Qiagen). Purified DNA is digested by SalI, then purified by Qiagen PCR purification Kit. Bisulfite conversion is carried out in accordance with standard procedures (Millar, Douglas S., et al. “Methylation sequencing from limiting DNA: embryonic, fixed, and microdissected cells.” Methods 27.2 (2002): 108-113). The converted DNA is amplified by PCR with primers specific for the bisulfite converted template. The amplified fragments are cloned into TOPO-TA vectors (Invitrogen Life Technology Inc.) and individual clones are used for sequencing.

### Example #3: Using a Targeted DNA to Methylate CpG Sites within the ELANE Promoter Region to Silence ELANE Gene Expression

Some individuals with severe congenital neutropenia (SCN) have autosomal dominant mutations in the ELANE gene, which causes the disease. This example describes a composition selected to target specific CpG-rich regions in an ELANE promoter. The prokaryotic C5-MTase M.HSssI and/or the eukaryotic Ct-MTase DNMT3A (described in Example 1) is/are split into two fragments (e.g. an N-terminal fragment and a C-terminal fragment), each fragment of which is, in turn, joined in vitro to a single ssDNA strand that targets a region within the ELANE promoter. The tethered MTase fragments join to form a catalytically active MTase that methylates CpG sites in the ELANE promoter to inhibit the ELANE gene.

Designing Guiding ssDNA Strands

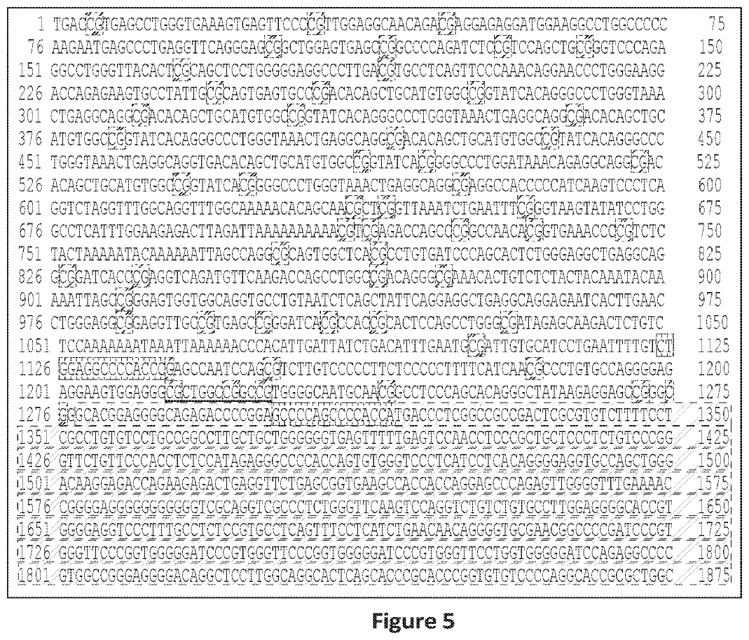

The ELANE promoter directly precedes the ELANE coding sequence and is found within the −1 and −1000 nucleotide positions with respect to the starting ATG codon. The ˜1 kb upstream region of the 5′ UTR is shown in FIG. 5 (highlighted in grey). CpG sites are highlighted in green and the target CpG-rich area is underlined. Using the same criteria as described in Example 1, targets for guiding ssDNA strands are chosen. DNA sequences targeted by the guiding ssDNA strands are highlighted in teal. The upstream guiding ssDNA strand has the sequence 5′ GACCTCCGGGGTGGG 3′, and the downstream guiding ssDNA strand has the sequence 5′ CGGGGTCGGGGTGGT 3′.

### Construction of Enzyme Fragment-ssDNA Fusions

Conjugation of the guiding ELANE ssDNA strands to the N- and C-terminal fragments of M.HSssI or DNMT3A catalytic domains described in Example 1 are performed using copper catalyzed click chemistry and an Oligo-click Kit (BaseClick). Reactions are performed in accordance with manufacturer's instructions, with a vial of catalyst beads, activator solution, the catalytic domain, and guiding ssDNA. Reactions are incubated in a Thermoshaker. Successful conjugation is determined by mass spectrometry.

### Cell Culture and Reporter Gene Assays

Human ELANE promoter (−1 to −1000 bp upstream of the start codon) is amplified from HEK293T genomic DNA and cloned into a pGL3-basic luciferase reporter gene vector (Promega). HEK293T cells are cultured in DMEM (PAA laboratories GmbH), supplemented with 10% fetal calf serum (FCS) (PAA laboratories GmbH). Briefly, the HEK293T cells are seeded in 24-well plates coated with polylysine. Plasmids for co-transfection, which include a GFP reporter gene, Renilla luciferase reporter gene controlled by cytomegalovirus (CMV) (Promega) and target firefly luciferase reporter gene, are diluted with serum free DMEM culture medium and mixed with Transfast™ reagent (Promega). Transfection is performed in accordance with manufacturer's instructions. Efficiency of transfection is monitored counting number of green fluorescent protein-positive (GFP+) cells under a fluorescence microscope (e.g. determining percentage of total number cells that are GFP+). e.

N- and C-terminal fragments of M.HSssI or DNMT3A, each conjugated to their respective guiding ssDNA strands, are then delivered to cells. Four days after delivery, the culture medium is removed and cells are lysed by adding lysis buffer from Renilla Luciferase Assay System (Promega, Cat. #E2810) to each well. Samples of crude cell lysate are transferred to different wells of a non-transparent micro well plate (Packard) for firefly and Renilla luciferase activity assay, respectively. Luciferase activity (luminescence signal) is determined by Topcount®NXT™ Microplate Scintillation & Luminescence Counter (Packard). In all transfection experiments, transfection yield and cell number are normalized by co-transfection with a construct expressing Renilla luciferase under the control of a CMV promoter. Luciferase activity, normalized by activity of the Renilla luciferase (not under control of PCSK9 promoter), is used as a read-out of promoter silencing.

### Bisulfite Analysis of ELANE Promoter Methylation

To analyze targeted DNA methylation, transfected HEK293T cells are harvested and washed with PBS. Episomal DNA is isolated using a Qiagen miniprep kit, following manufacturer protocol. Cells are harvested and total cellular DNA is isolated using DNeasy Tissue Kit (Qiagen). Purified DNA is digested by SalI, then purified by Qiagen PCR purification Kit. Bisulfite conversion is carried out as the standard procedure (Millar, Douglas S., et al. “Methylation sequencing from limiting DNA: embryonic, fixed, and microdissected cells.” Methods 27.2 (2002): 108-113). The converted DNA is amplified by PCR with primers specific for the bisulfite converted template. The amplified fragments are cloned into TOPO-TA vectors (Invitrogen Life Technology Inc.) and individual clones are used for sequencing.

### Example #4: Designing Tet1 Fragments

Using sequence homology to Tet2, the catalytic domain of Tet1 (residues 1418-2136, derived from the plasmid pJFA344C7 (Addgene plasmid: 49236) or Uniprot: Q8NFU7) is split into two fragments, designed such that each fragment is catalytically inactive on its own, but upon binding to one another a functional catalytic complex is formed. The following two fragments are designed: an N-terminal fragment having residues 1418-1845, and a C-terminal fragment having residues 1846-2136.

### Example #5: Using Tet1 to Demethylate CpG Sites within the FMR1 Promoter Region to Restore FMRP Expression

Hypermethylation of CpG sites (CpG islands) in the promoter sequence of many genes leads to aberrant suppression of gene expression. One such example is the existence of CpG islands in the gene FMR1, leading to suppression of expression of FMRP and Fragile X Syndrome.

This example describes a composition designed to target specific CpG-rich regions or CpG islands in the FMR1 promoter region. The eukaryotic protein Tet methylcytosine dioxygenase 1 (Tet1) is responsible for catalyzing the initial step of cytosine demethylation. Tet1 is split into two fragments (e.g. an N-terminal fragment and a C-terminal fragment), each of which is, in turn, joined in vitro to a single ssDNA strand that targets a region within an FMR1 promoter. Each of the fragments is catalytically inactive (or effectively inactive) on its own, but upon binding to one another a catalytically active enzyme is formed. The ssDNA sequences serve as a targeting mechanism that pairs with a promoter region of the FMR1 gene, thereby directing the tethered fragments to that particular genomic location. When bound to their respective target regions in the FMR1 promoter, the guiding ssDNA strands serve as tethers that allow interaction between the two Tet1 fragments and formation of a catalytically active enzyme. Applicant proposes that the targeting mechanism of the guiding ssDNA strands further restricts the catalytically active enzyme to demethylate nearby CpG sites.

Designing Guiding ssDNA Strands

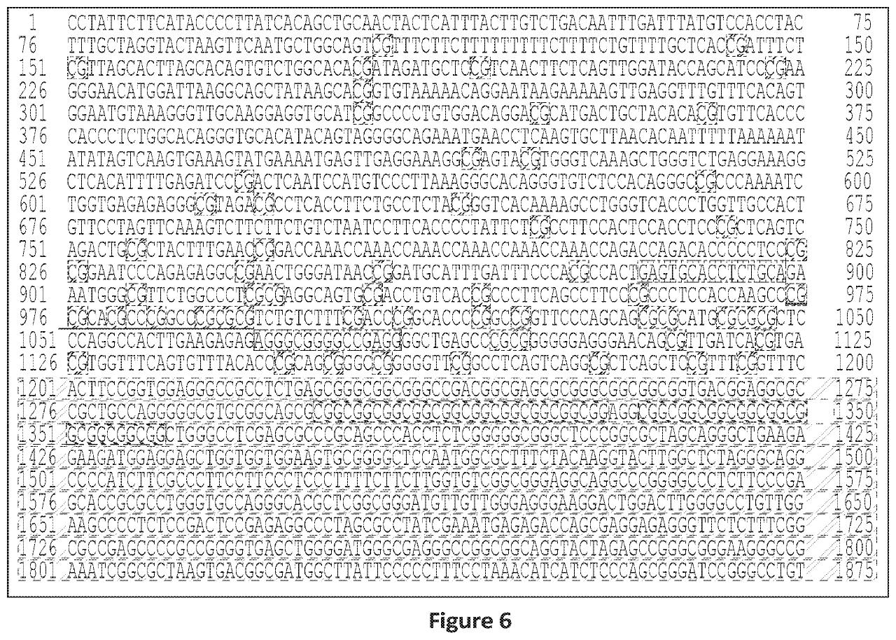

The FMR1 promoter directly precedes FMR1's coding sequence and is found within the −1 and −1000 nucleotide positions with respect to the starting ATG codon. The ˜1 kb upstream region of the 5′ UTR is shown in FIG. 6 (highlighted in grey). CpG sites are highlighted in green and the target CpG-rich area is underlined. Using the same criteria as described in Example 1, targets for the guiding ssDNA strands are chosen. The DNA sequences targeted by the guiding ssDNA strands are highlighted in teal. The upstream guiding ssDNA strand has the sequence 5′ CTCACGTGGAGACGT 3′, and the downstream guiding ssDNA strand has the sequence 5′ TCCCGCCCCGGCTCC 3′.

### Construction of Enzyme Fragment-ssDNA Fusions

Conjugation of the guiding ssDNA strands to the N- and C-terminal fragments of Tet1 catalytic domains described in Example 4 are performed using copper catalyzed click chemistry and an Oligo-click Kit (BaseClick). Reactions are performed in accordance with the manufacturer's instructions, with a vial of catalyst beads, activator solution, the catalytic domain, and guiding ssDNA. Reactions are incubated in a Thermoshaker. Successful conjugation is determined by mass spectrometry.

### Cell Culture and Reporter Gene Assays

Human FMR1 promoter (˜1000 bp) is amplified from HEK293T genomic DNA and cloned into a pGL3-basic luciferase reporter gene vector (Promega). HEK293T cells are cultured in DMEM (PAA laboratories GmbH), supplemented with 10% fetal calf serum (FCS) (PAA laboratories GmbH). Briefly, the HEK293T cells are seeded in 24-well plates coated with polylysine. Plasmids for co-transfection, which include a GFP reporter gene, Renilla luciferase reporter gene controlled by cytomegalovirus (CMV) (Promega), and target firefly luciferase reporter gene, are diluted with serum free DMEM culture medium and mixed with Transfast™ reagent (Promega). Transfection is performed in accordance with manufacturer's instructions. Efficiency of transfection is monitored by green fluorescent protein (GFP)+ cells under a fluorescence microscope.

N- and C-terminal fragments of Tet1, each conjugated to its respective guiding ssDNA strands, are then delivered to cells. Four days after delivery, the culture medium is removed and cells are lysed by adding lysis buffer from Renilla Luciferase Assay System (Promega, Cat. #E2810) to each well. Samples of crude cell lysate are transferred to different wells of a non-transparent micro well plate (Packard) for firefly and Renilla luciferase activity assay, respectively. Luciferase activity (luminescence signal) is determined by Topcount®NXT™ Microplate Scintillation & Luminescence Counter (Packard). In all transfection experiments, transfection yield and cell number are normalized by co-transfection with a construct expressing Renilla luciferase under the control of a CMV promoter. Luciferase activity, normalized by activity of the Renilla luciferase (not under control of PCSK9 promoter), is used as a read-out of promoter silencing.

### Bisulfite Analysis of FMR1 Promoter Methylation

To analyze targeted DNA methylation, transfected HEK293T cells are harvested and washed with PBS. Episomal DNA is isolated using a Qiagen miniprep kit, following manufacturer protocol. Cells are harvested and total cellular DNA is isolated using DNeasy Tissue Kit (Qiagen). Purified DNA is digested by SalI, then purified by Qiagen PCR purification Kit. Bisulfite conversion is carried out as the standard procedure (Millar, Douglas S., et al. “Methylation sequencing from limiting DNA: embryonic, fixed, and microdissected cells.” Methods 27.2 (2002): 108-113). The converted DNA is amplified by PCR with primers specific for the bisulfite converted template. The amplified fragments are cloned into TOPO-TA vectors (Invitrogen Life Technology Inc.) and individual clones are used for sequencing.

### Example #6: Designing Cpf1 Fragments

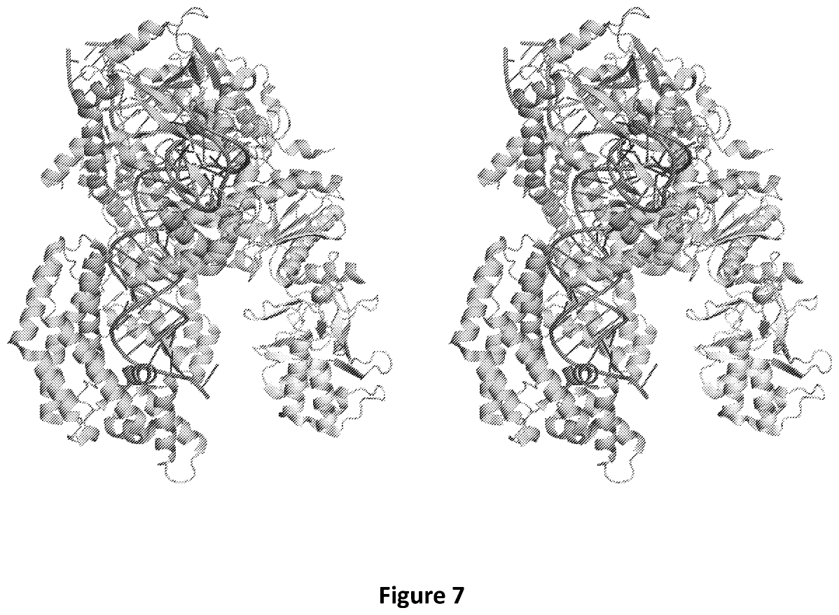

Cpf1 endonucleases recognize T-rich PAM sites, e. g., 5′-TTN, as well as the 5′-CTA PAM motif. Cpf1 cleaves target DNA by introducing an offset or staggered double-strand break. Cpf1 consists of two lobes (full length AsCpf1 (residues 1-1307) is derived from pCAG-GFP (addgene: 78743) or Uniprot: U2UMQ6), the REC lobe (resides 24-525) and the NUC lobe (residues 1-23 and 526-1307). Endonuclease activity is contained within the NUC lobe, with the RuvC domain (residues 864-1066 and 1262-1307) responsible for cleaving the non-target strand and the Nuc domain (residues 1066-1262) responsible for cleaving the target-strand. Cpf1 is split into two fragments, designed such that each fragment is catalytically inactive on its own, but upon binding to one another a functional catalytic complex is formed. The following two fragments are designed: an N-terminal fragment having residues 1-1066, and a C-terminal fragment having residues 1067-1307 (FIG. 7).

### Example #7: Using Cpf1 to Delete a MYC Binding Site within the BCR Promoter to Silence BCR-ABL Expression

BCR-ABL is a gene formed as a result of reciprocal translocation of pieces of chromosomes 9 and 22. The ABL gene from chromosome 9 joins to the BCR gene on chromosome 22, to form the BCR-ABL fusion gene. The BCR-ABL fusion gene is found in most patients with chronic myelogenous leukemia (CML), and in some patients with acute lymphoblastic leukemia (ALL) or acute myelogenous leukemia (AML). The BCR-ABL fusion gene is controlled by a BCR promoter. MYC is a transcription factor that upregulates expression of the BCR-ABL fusion. Deleting MYC binding sites within the BCR promoter region has been shown to silence BCR-ABL gene expression.

This example describes a composition selected to delete a specific MYC binding site within the BCR promoter region. In certain Examples as described herein, Cpf1 is responsible for excision of a target site. Cpf1, as described in Example 6, is split into two fragments (e.g. an N-terminal fragment and a C-terminal fragment), each of which is, in turn, joined in vitro to a single ssDNA strand that targets a region within the BCR promoter. The tethered Cpf1 fragments join to form a catalytically active nuclease that cleaves BCR promoter to inhibit BCR-ABL gene expression.

Designing Guiding ssDNA Strands

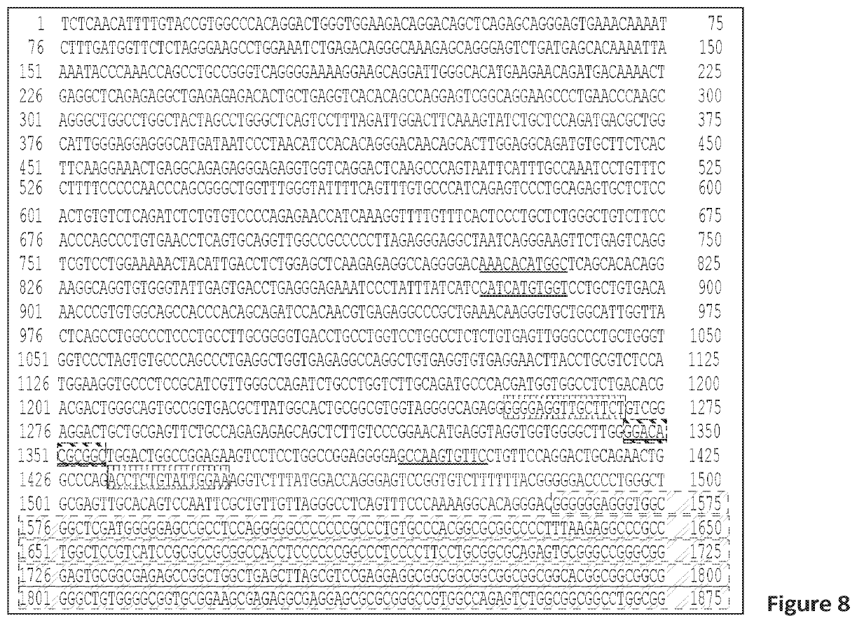

The ˜1 kb upstream region of the BCR 5′ UTR is shown in FIG. 8 (highlighted in grey), with MYC binding sites within the BCR promoter underlined. The third MYC binding site (highlighted in purple) is selected for deletion. Using the same criteria as described in Example 1, targets for the guiding ssDNA strands are chosen. The DNA sequences targeted by the guiding ssDNA strands are highlighted in teal. The upstream guiding ssDNA strand has the sequence 5′ CCCCTCCAACGAAGA 3′, and the downstream guiding ssDNA strand has the sequence 5′ TGGAGACATAACCTT 3′.

### Construction of Enzyme Fragment-ssDNA Fusions

Conjugation of the guiding ssDNA strands to the N- and C-terminal fragments of Cpf1 catalytic domains described in Example 6 is performed using copper catalyzed click chemistry and an Oligo-click Kit (BaseClick). Reactions are performed in accordance with the manufacturer's instructions, with a vial of catalyst beads, activator solution, the catalytic domain, and guiding ssDNA. Reactions are incubated in a Thermoshaker. Successful conjugation is determined by mass spectrometry.

### Cell Culture and Reporter Gene Assays

Human BCR promoter (−1500 to −1 relative to start codon) is amplified from HEK293T genomic DNA and cloned into a pGL3-basic luciferase reporter gene vector (Promega). HEK293T cells are cultured in DMEM (PAA laboratories GmbH), supplemented with 10% fetal calf serum (FCS) (PAA laboratories GmbH). Briefly, the HEK293T cells are seeded in 24-well plates coated with polylysine. Plasmids for co-transfection, which include a GFP reporter gene, Renilla luciferase reporter gene controlled by cytomegalovirus (CMV) (Promega), and target firefly luciferase reporter gene, are diluted with serum free DMEM culture medium and mixed with Transfast™ reagent (Promega). Transfection is in accordance with manufacturer's instructions. Efficiency of transfection is monitored by counting number of green fluorescent protein-positive (GFP+) cells under a fluorescence microscope (e.g. determining percentage of total number cells that are GFP+).

N- and C-terminal fragments of Cpf1, each conjugated to its respective guiding ssDNA strands, are then delivered to cells. Four days after delivery, the culture medium is removed and cells are lysed by adding lysis buffer from Renilla Luciferase Assay System (Promega, Cat. #E2810) to each well. Samples of crude cell lysate are transferred to different wells of a non-transparent micro well plate (Packard) for firefly and Renilla luciferase activity assay, respectively. The luciferase activity (luminescence signal) is determined by Topcount®NXT™ Microplate Scintillation & Luminescence Counter (Packard). In all transfection experiments, transfection yield and cell number are normalized by co-transfection with a construct expressing Renilla luciferase under the control of a CMV promoter. Luciferase activity, normalized by activity of the Renilla luciferase (not under control of PCSK9 promoter), is used as a read-out of promoter silencing.

### Example #8: Designing DRM2 Fragments

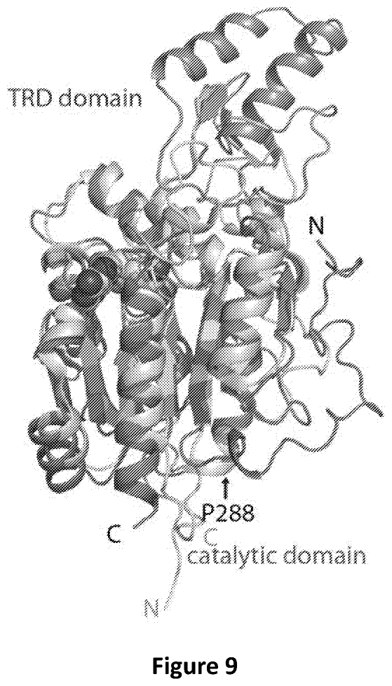

Unlike in mammals where DNA methylation predominantly occurs in CG context, plant DNA is frequently methylated in three different sequence contexts: CG, CHG and CHH (H=A, T, or C). In Arabidopsis thaliana, the maintenance of CG methylation is primarily controlled by MET1 (an ortholog of mammalian DNMT1), while DRM2 (an ortholog of mammalian DNMT3) is responsible for both de novo methylation as well as maintenance of CHH. The structure of the A. thaliana DRM2 is informed by the structure of the related DRM1 from Nicotiana tabacum. The catalytic domain of DRM2 shows sequence and structural similarity to those of the DNMT3 methyltransferases (see FIG. 9).

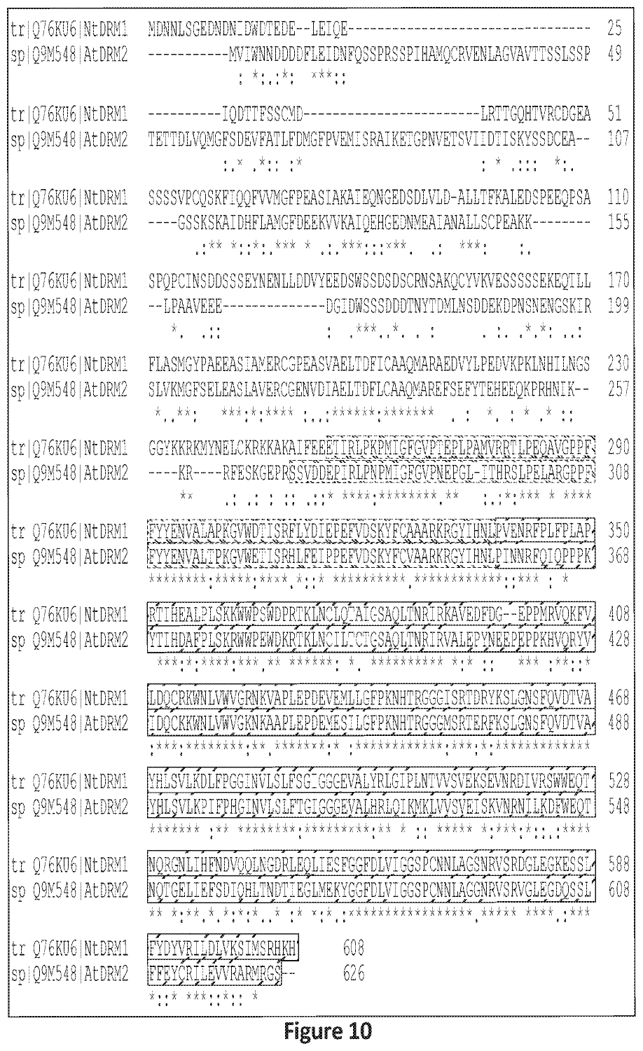

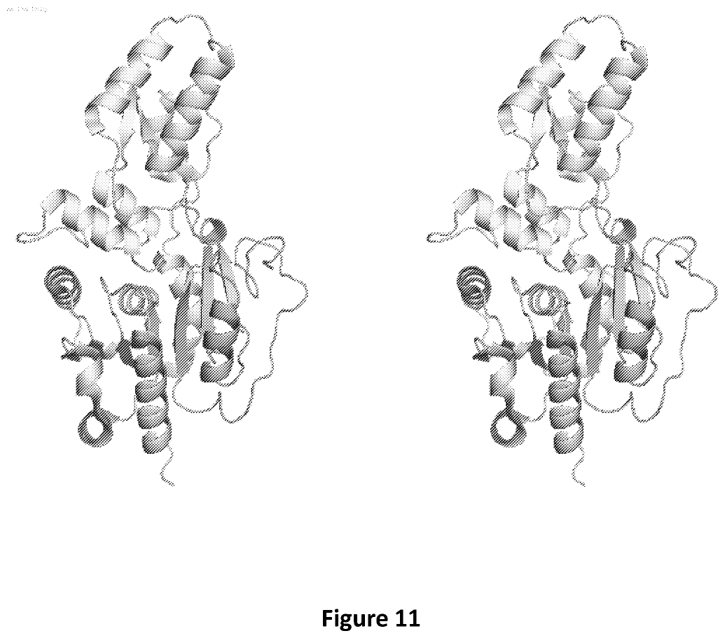

Given the sequence and structural similarities to the N. tabacum DRM1 and eukaryotic DNMT3A, the catalytic domain of A. thaliana DRM2 (residues 269-621) is split into two fragments, designed such that each fragment is catalytically inactive on its own, but upon binding to one another a functional catalytic complex is formed. The following two fragments are designed: an N-terminal fragment having residues 269-355, and a C-terminal fragment having residues 356-626 (see FIGS. 10 and 11).

### Example #9: Using DRM2 to Methylate CpG Sites within the FWA Promoter, Thereby Silencing FWA Expression and Preventing a Late Flowering Phenotype

Induction of flowering at an appropriate moment is essential for many plant species to reproduce successfully. Fine-tuning a transition from vegetative to reproductive phase is under the control of multiple factors, e.g., by regulating gene expression affecting flowering transition through DNA methylation.

Plants treated with a DNA demethylating agent, 5-azacytidine, are hypomethylated and tend toward late flowering when compared to untreated plants. The late flowering trait maps to the chromosomal region containing FWA that encodes a homeodomain-containing transcription factor that controls flowering. FWA is presumed to affect flowering through the speculated photoperiod promotion pathway in a current model for control of flowering initiation. FWA is normally silenced in wild-type plants, with reversal leading to plants with a late flowering phenotype. The FWA gene contains two tandem repeats around the transcription start site that are necessary and sufficient for silencing via DNA methylation.

This example describes a composition selected to methylate a specific pair of CpG sites within a tandem repeat found in the FWA promoter region. The protein DRM2 is responsible for methylation of the target CpG sites, and here is split into two fragments (e.g. an N-terminal fragment and a C-terminal fragment), each of which is, in turn, joined in vitro to a single ssDNA strand that targets a tandem repeat region within the FWA promoter. Each of the fragments is on its own catalytically inactive (or effectively inactive), but upon binding to one another a catalytically active enzyme is formed. The ssDNA sequences serve as a targeting mechanism that pairs with a promoter region of the FWA gene, thereby directing the tethered fragments to that particular genomic location. When bound to their respective target regions in the FWA promoter, the guiding ssDNA strands serve as tethers that allow interaction between the two DRM2 fragments and formation of a catalytically active enzyme. Applicant proposes that the targeting mechanism of the guiding ssDNA strands further restricts the catalytically active enzyme to methylate nearby CpG sites.

Designing Guiding ssDNA Strands

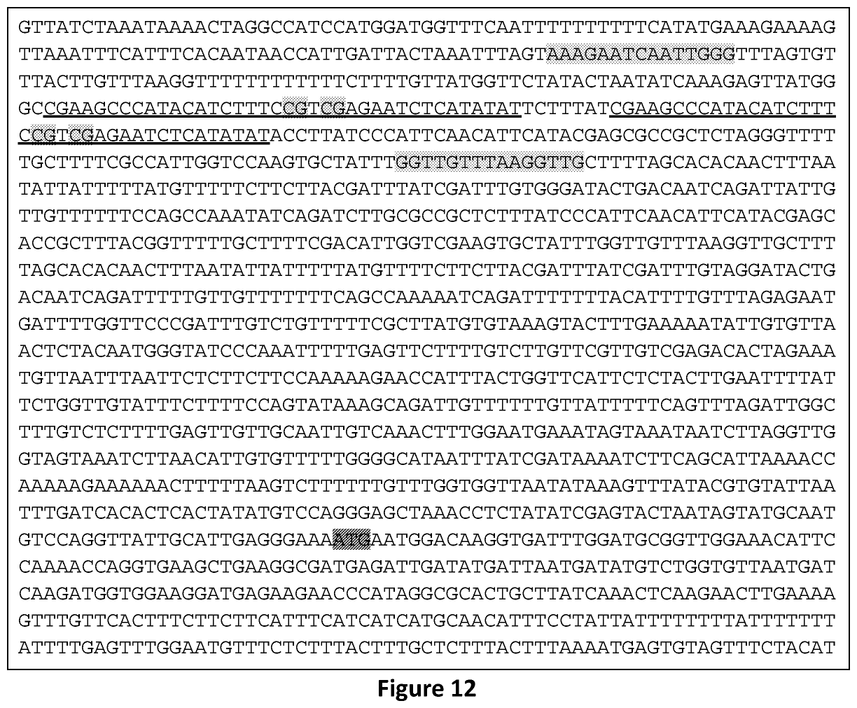

The 2.4 kb upstream region of the FWA start codon is shown in FIG. 12, with one of the tandem repeat pairs underlined. The CpG sites within this tandem repeat whose methylation leads to FWA silencing are highlighted in green. Using the same criteria as described in Example 1, targets for the guiding ssDNA strands are chosen. The upstream guiding ssDNA strand has the sequence 5′ TTTCTTAGTTAACCC 3′, and the downstream guiding ssDNA strand has the sequence 5′ CCAACAAATTCCAAC 3′.

### Plant Materials, Growth Conditions, and Measurement of Flowering Time

Isolation of ddm1 mutants from Arabidopsis is as reported by Vongs et al. (1993). The ddm1-1 allele in the Columbia (Col) background is used throughout. The ddm1-1 mutants and wild-type genotypes are distinguished by examining PCR products with primer pairs 5′-ATTTGCTGATGACCAGGTCCT-3′ and 5′-CATAAACCAATCTCATGAGGC-3′, and restriction digestion by NsiI.

Plants are grown either in a greenhouse with LD light regime (at least 14 hr day length) or in a climate chamber with SD light conditions (8 hr of light per day) as described in Koornneef et al., Physiologia Plantarum 95.2 (1995): 260-266. Flowering time is measured by counting the total number of leaves, excluding the cotyledons, since there is a close correlation between leaf number and flowering time (Koornneef et al., Molecular and General Genetics MGG 229.1 (1991): 57-66).

### Analysis of RNA and Genomic DNA

For FWA expression analysis, RNA is prepared using the RNeasy Plant Mini Kit (Qiagen, Hilden, Germany). RT-PCR is performed using the RETROscript kit (Ambion, St. Austin, Tex., USA) or One Step RNA kit (Takara, Ohtsu, Japan). In short, after reverse transcription, cDNA from input RNA is amplified in 25 PCR cycles and detected by electrophoresis. To detect FWA and control GAPC transcript, primer pairs 5′-GCTCACTCCAACAGATTCAAGCAG-3′ and 5′-GTTGGTAGATGAAAGGGTCGAGAG-3′; and 5′-CACTTGAAGGGTGGTGCCAAG-3′ and 5′-CCTGTTGTCGCCAACGAAGTC-3′, respectively, are used. Products from genomic DNA and mRNA can be distinguished by size as the intron is included within the amplified region. Southern analysis of genomic DNA is performed as described previously (Miura et al., Molecular Genetics and Genomics 270.6 (2004): 524-532).

### Detection of DNA Methylation by the Bisulfite Method

Bisulfite sequencing is performed as described by Paulin et al., Nucleic acids research 26.21 (1998): 5009-5010. After the chemical bisulfite reaction, PCR fragments overlapped to regions A-C are amplified with the following primers. For the A region, 5′-AGGTTYTYATYATATAYYGAAAGAATGGGA-3′ and 5′-TTRAAACCATCCATRRATRRCCTARTT-3′; B region, 5′-AAAGAGTTATGGGYYGAAG-3′ and 5′-CRRRAACCAAAATCATTCTCTAAACA-3′; C region, 5′-TGTTTAGAGAATGATTTTGGTTYYYG-3′ and 5′-CTACCAACCTAARATTATTTACTATTTCATTCCAA-3′. The amplified PCR fragments are gel-purified and cloned into pT7Blue plasmid (Novagen, San Diego, Calif., USA), and then 10-12 independent clones are sequenced. The ASA1 gene (Jeddeloh et al., Genes & Development 12.11 (1998): 1714-1725) is used as a positive control for the bisulfite chemical reaction.

### Example #10: Designing DME Fragments

In plants, the DEMETER (DME) family of DNA glycosylases functions to remove 5mC, which is then replaced by unmethylated cytosine, resulting in transcriptional activation of target genes. DME (Uniprot: Q8LK56) family DNA glycosylases have both common and unique structural and functional features compared to typical DNA glycosylases. The glycosylase domain of DME contains a helix-hairpin-helix (HhH) motif and a glycine/proline-rich loop with a conserved aspartic acid (GPD), also found in human 8-oxoguanine DNA glycosylase (hOGG1), Escherichia coli adenine DNA glycosylase (MutY), and endonuclease III (Endo III). In contrast to most other members of the HhH glycosylase superfamily, DME family members contain two additional conserved domains that flank the central glycosylase domain: domain A (residues 690-797) and domain B (1448-1720). The interdomain regions are poorly conserved and have been shown to be dispensable for catalytic activity, as have the N-terminal 677 residues (residues 1-677 of the N-terminal region).

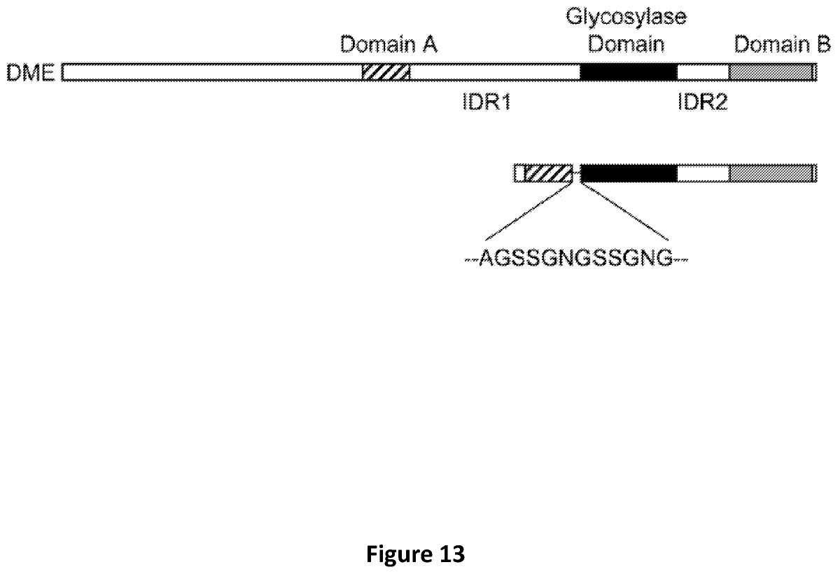

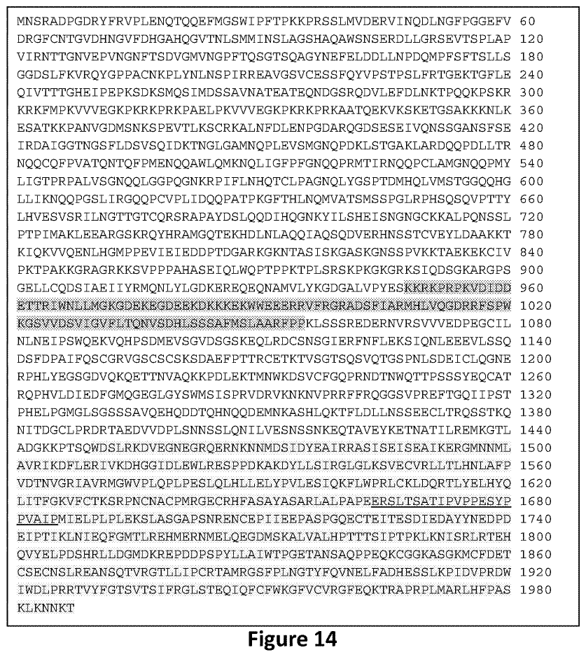

Biochemical experiments have identified three domains within the Arabidopsis thaliana DME protein that are sufficient and necessary for catalytic activity (see FIGS. 13 and 14). A minimum construct consisting of the following five elements has been shown to retain catalytic activity: domain A (residues 948-1055), the artificial linker sequence AGSSGNGSSGNG, the glycosylase domain (residues 1450-1663), the interdomain region 2 (residues 1664-1705), and finally domain B (residues 1706-1978)).

This minimum catalytic domain of A. thaliana DME is split into two fragments, designed such that each fragment is catalytically inactive on its own, but upon binding to one another a functional catalytic complex is formed. The following two fragments are designed: an N-terminal fragment having residues 948-1055, and a C-terminal fragment having residues 1450-1978 (see FIG. 14).

### Example #11: Using DME to Demethylate CpG Sites within the FWA Promoter, Thereby Enhancing FWA Expression and Inducing a Late Flowering Phenotype

This example describes a composition selected to demethylate a specific pair of CpG sites within a tandem repeat found in the FWA promoter region. The protein DME is responsible for demethylation of the target CpG sites, and here is split into two fragments (e.g. an N-terminal fragment and a C-terminal fragment), each of which is, in turn, joined in vitro to a single ssDNA strand that targets a tandem repeat region within the FWA promoter. Each of the fragments is catalytically inactive (or effectively inactive) on its own, but upon binding to one another, a catalytically active enzyme is formed. The ssDNA sequences serve as a targeting mechanism that pairs with a promoter region of the FWA gene, thereby directing the tethered fragments to that particular genomic location. When bound to their respective target regions in the FWA promoter, the guiding ssDNA strands serve as a tether that allows interaction between the two DME fragments and the formation of a catalytically active enzyme. Applicant proposes that the targeting mechanism of the guiding ssDNA strands further restricts the catalytically active enzyme to demethylate nearby CpG sites.

Designing Guiding ssDNA Strands

The 2.4 kb upstream region of the FWA start codon is shown in FIG. 12, with one of the tandem repeat pairs underlined. The CpG sites within this tandem repeat whose methylation leads to FWA silencing are highlighted in green. Using the same criteria as described in Example 1, targets for the guiding ssDNA strands are chosen. The upstream guiding ssDNA strand has the sequence 5′ TTTCTTAGTTAACCC 3′, and the downstream guiding ssDNA strand has the sequence 5′ CCAACAAATTCCAAC 3′.

### Plant Materials, Growth Conditions, and Measurement of Flowering Time

Isolation of ddm1 mutants from Arabidopsis is performed as previously reported (Vongs et al., Science 260.5116 (1993): 1926-1929). The ddm1-1 allele in the Columbia (Col) background is used throughout. The ddm1-1 mutants and wild-type genotypes are distinguished by examining PCR products with primer pairs 5′-ATTTGCTGATGACCAGGTCCT-3′ and 5′-CATAAACCAATCTCATGAGGC-3′, and restriction digestion by NsiI.

Plants are grown either in a greenhouse with LD light regime (at least 14 hr day length) or in a climate chamber with SD light conditions (8 hr of light per day) as described in Koornneef et al., Physiologia Plantarum 95.2 (1995): 260-266. Flowering time is measured by counting total number of leaves, excluding cotyledons, since there is a close correlation between leaf number and flowering time Koornneef et al., Molecular and General Genetics MGG 229.1 (1991): 57-66.

### Analysis of RNA and Genomic DNA

For FWA expression analysis, RNA is prepared using the RNeasy Plant Mini Kit (Qiagen, Hilden, Germany). RT-PCR is performed using the RETROscript kit (Ambion, St. Austin, Tex., USA) or One Step RNA kit (Takara, Ohtsu, Japan). In short, after reverse transcription, cDNA from input RNA is amplified in 25 PCR cycles and detected by electrophoresis. To detect FWA and control GAPC transcript, primer pairs 5′-GCTCACTCCAACAGATTCAAGCAG-3′ and 5′-GTTGGTAGATGAAAGGGTCGAGAG-3′ and 5′-CACTTGAAGGGTGGTGCCAAG-3′ and 5′-CCTGTTGTCGCCAACGAAGTC-3′, respectively, are used. Products from genomic DNA and mRNA can be distinguished by size as the intron is included within the amplified region. Southern analysis of genomic DNA is performed as described previously (Miura, A., et al., Molecular Genetics and Genomics 270.6 (2004): 524-532).

### Detection of DNA Methylation by the Bisulfite Method

Bisulfite sequencing is performed as described by Paulin et al., Nucleic acids research 26.21 (1998): 5009-5010. After the chemical bisulfite reaction, PCR fragments overlapped to regions A-C are amplified with the following primers. For the A region, 5′-AGGTTYTYATYATATAYYGAAAGAATGGGA-3′ and 5′-TTRAAACCATCCATRRATRRCCTARTT-3′; B region, 5′-AAAGAGTTATGGGYYGAAG-3′ and 5′-CRRRAACCAAAATCATTCTCTAAACA-3′; C region, 5′-TGTTTAGAGAATGATTTTGGTTYYYG-3′ and 5′-CTACCAACCTAARATTATTTACTATTTCATTCCAA-3′. The amplified PCR fragments are gel-purified and cloned into pT7Blue plasmid (Novagen, San Diego, Calif., USA), and then 10-12 independent clones are sequenced. The ASA1 gene (Jeddeloh et al., Genes & Development 12.11 (1998): 1714-1725) is used as a positive control for the bisulfite chemical reaction.

### Example #12: Engineered Split Effector Moieties

In this Example, certain engineered split effector moieties are provided. Specifically, fragments from a protein (e.g. effector) entity, which may, for example, be a naturally-occurring protein that, in nature, is encoded as a single polypeptide chain and possessing a specific biochemical activity (e.g. interaction with specific proteins, catalysis of chemical molecule conversions, catalysis of post-translational modifications, transport of molecules across membranes) are designed as at least two separate fragments (i.e. a first fragment and a second fragment, e.g. a full-length protein entity is “split” into fragments). Each engineered fragment alone has minimal specific biochemical activity as compared to the corresponding full-length protein (e.g. effector) entity, encoded as a single polypeptide chain; in some cases, a specific biochemical activity comparable (e.g., equivalent) to the full length protein (e.g. effector) entity is achieved (e.g. by forming appropriate molecular interactions) upon the at least two fragments, when co-localized (e.g., by delivery to a target genomic location.

As described in this particular Example, targeting of the protein (e.g. effector) entity fragments to a specific genomic location is accomplished by associating (e.g., covalently linking) each effector entity fragment with a separate targeting moiety, which separate targeting moieties each localize its fragment to the same chromosomal location, thereby permitting association of the split effector moiety fragments and formation (e.g., reconstitution) of an active effector moiety.

In a particular split effector moiety system as described in this Example, a first fragment includes a targeting moiety that binds to a specific genomic site, and the second fragment includes a targeting moiety that binds to endogenous DNA or histones, which are or can be co-localized in three-dimensional space (and, optionally, linearly along a particular chromosome) with the specific genomic site. In some embodiments of this particular example, the targeting moiety is a guide RNA (gRNA) complexed with either Cas9 or a mutated form of Cas9. Targeting at the intended genomic location is considered to be likely to result in modulating of one or more particular targeted genomic sites. Without wishing to be bound by any particular theory, Applicants propose that modulation of a targeted genomic location may occur by, e.g. facilitating interaction of the two fragments at the targeted genomic location and/or resulting in reconstituted specific biochemical activity equivalence to that of the full length effector entity at or in proximity to the targeted genomic location.

### Example #12.1: Split Effector Moieties for Epigenetic Modifications (TUSC5)

This Example describes two engineered fragments of human DNMT3L protein. A first fragment is engineered such that it is capable of binding chromatin with unmethylated histone H3 lysine 4 (H3K4me0) and a second fragment is engineered by fusion to a targeting moiety via covalently tethering (e.g., fused) to a mutated Cas9 protein (Cas9 protein with D10A and H840A mutations; “dCas9”); these entities are referred to DNMT3L_fragment1 and DNMT3L_fragment2::dCas9.

As will be appreciated by one of skill in the art, human DNMT3L protein is an essential regulator of human DNMT3A protein, a DNA methyltransferase. DNMT3L can directly bind to chromatin with unmethylated histone H3 lysine 4 (H3K4me0) and can induce de novo DNA methylation by recruitment and activation of DNMT3A.

This Example demonstrates disruption of TUSC5 gene-associated genomic location by epigenetic modification. TUSC5 is located with a particular genomic location (“TUSC5 target genomic location”). In HEK293T cells, TUSC5 is not expressed, and there are multiple active enhancers outside this target genomic location, both upstream and downstream. Disruption of CTCF binding sites at either end of the TUSC5 target genomic location is considered to be likely to cause the enhancers outside the target genomic location to activate expression of TUSC5.

Targeting of DNMT3L_fragment2::dCas9 to TUSC5 gene-associated genomic location is considered to be likely to result in methylation of cytosine bases at or in proximity the TUSC5 gene associated genomic location, reduced CTCF occupancy at the targeted genomic location, and/or increased expression of TUSC5. In particular and without wishing to be bound by any particular theory, Applicant proposes that targeting of DNMT3L_fragment2::dCas9 to TUSC5 gene-associated genomic location is considered to be likely to reconstitute biochemical activity (e.g. methylation of cytosine bases in genomic DNA) at the targeted location by binding to DNMT3L_fragment1; the reconstituted biochemical activity is comparable to that of full-length DMNT3L::dCas9 protein when targeted to the same location (e.g. appropriate gRNAs).

Production of Split Effector Moieties and Associated Components

All plasmids and guide RNAs (gRNA) are chemically synthesized from commercially available vendors. All agents are reconstituted in sterile water. Three plasmids (“Plasmid 1”; “Plasmid 2”; and “Plasmid 3”) are synthesized and each contains a dCas9 expression cassette, where dCas9 expression is driven by CMV enhancer and chicken beta-actin promoter with an SV40 nuclear localization sequence (NLS) on the N-terminus and a C-terminal linker

(cctgcttctggcggaacttcatctgatggtggcacgtcagacgga
gggtcaagcaacacaggcggtagctctgacggagggagctcagaag
gcgaacctgcgcatgca).

Plasmid 1

In plasmid 1, the sequence of full length human DNMT3L (UniProtKB—Q9UJW3) with C-terminal SV40NLS follows the 3′ end of the C-terminal linker.

Plasmid 2

In plasmid 2, the sequence of human DNMT3L_fragment1 for split construct 1, as listed in Table 1, with C-terminal SV40NLS follows the 3′ end of the C-terminal linker. In addition, in plasmid 2, the sequence of human DNMT3L_fragment2 for split construct 1, as listed in Table 1, is driven by an IRES promoter and has both N and C-terminal SV40 NLSes.

Plasmid 3

In plasmid 3, the sequence of human DNMT3L_fragment1 for split construct 2, as listed in Table 1, with C-terminal SV40NLS follows the 3′ end of the C-terminal linker. In addition, in plasmid 3, the sequence of human DNMT3L_fragment2 for split construct 2, as listed in Table 1, is driven by an IRES promoter and has both N and C-terminal SV40 NLSes.

TABLE 1
Design of DNMT3L split constructs.

| | DNMT3L Fragment 1 | DNMT3L Fragment 2 |
|---|---|---|
| Split construct 1 | DNMT3L amino acids | DNMT3L amino acids |
| | 179-354 | 354-358 |
| Split construct 2 | DNTM3L amino acids | DNMT3L amino acids |
| | 179-330 | 331-378 |

TABLE 2
Sequences of gRNAs targeting
putative CTCF sites of TUSC5-associated
genomic locations.

| ID | Guide RNA Sequence (5′-3′) |
|---|---|
| SACR-00214 | CAGCGGATTTGGGCTCCCGG |
| SACR-00216 | CCTCATCACTACCTGCCACG |
| SACR-00217 | CATCACTACCTGCCACGAGG |
| SACR-00218 | TGAGACTCCAGCATCCCACA |
| SACR-00219 | CCAGAGTAGTCCCTGGCACG |

Exemplary plasmids are listed in Table 1 and described herein. HTEK293T cells are serially transfected (either with a first plasmid, then a second plasmid, or with a second plasmid and then a first plasmid) with a first plasmid encoding DNMT3L_fragment1 and a second plasmid encoding DNMT3L_fragment2::dCas9 or, alternatively and/or additionally with a plasmid encoding DNMT3L(full length)::dCas9 and either a non-targeting gRNA (“Non-targeting,” where the guide RNA sequence has no homology to the human genome) or a gRNA, as listed in Table 2, targeted at or near the putative CTCF binding sequence of the targeted TUSC5-associated genomic location and/or full length DNMT3L and at least one fragment, each tagged with different epitopes to facilitate distinguishing occupancy during, e.g. a competitive binding experiment. HEK293T cells are transfected with a plasmid encoding the DNMT3L_fragment2::dCas9, and then transfected, 8 hours later, with either a chemically synthesized gRNA targeting the target genomic location, or a non-targeting gRNA.

At 72 hours post-transfection, cells are harvested for RNA extraction and cDNA synthesis using commercially available reagents and protocols (Qiagen; Thermo Fisher Scientific) and genomic DNA is extracted (Qiagen). The resulting cDNA is used for quantitative real-time PCR (Thermo Fisher Scientific). TUSC5-specific quantitative PCR probes/primers (Thermo Fisher Scientific) are multiplexed with internal control quantitative PCR probes/primers for PPIB (Assay ID Hs00168719_m1, Thermo Fisher Scientific) using FAM-MGB and VIC-MGB dyes, respectively, and gene expression is subsequently analyzed by a real time PCR kit (Applied Biosystems, Thermo Fisher Scientific).

Cells transfected with split effector entities, DNMT3L_fragment1 and DNMT3L_fragment2::dCas9 (with DNMT3L_fragment2::dCas9 targeted to a TUSC5 gene-associated genomic location as described herein) is considered to be likely to show increases in TUSC5 expression as compared to non-targeting controls.

To analyze DNA methylation, extracted genomic DNA is bisulfite converted and purified using commercially available reagents and protocols (Qiagen). Bisulfite-converted genomic DNA is used as template to amplify the CTCF-binding DNA region (primers are designed to amplify chr17:1179320-1179846 of human hg19 genome) and multiple non-targeting DNA regions by a PCR kit (New England Biolabs). CpG methylation is determined by sequencing the resultant PCR products. By aligning sequences of the resultant PCR products to the unconverted reference DNA sequence, unmethylated CpGs are identified by thymidine (“T”) base calls where “T” is sequenced in place of cytosine (“C”). Thus, CpG methylation is represented by any number of non-zero “C” base calls followed by guanosine (“G”).

The degree of split effector entity mediated CpG methylation (e.g. by interactions of, DNMT3L_fragment1 and DNMT3L_fragment2::dCas9) is subsequently ascertained by comparing number and position of “C” base calls in the TUSC5-targeted samples as compared to the non-targeting control and/or as compared to cells transfected with DNMT3L (full length)::dCas9 and targeting gRNAs, where an integer increase in “C” base calls indicates split effector entity targeted CpG methylation.

Cells transfected with split effector entities, DNMT3L_fragment1 and (with targeted to a TUSC5 gene associated genomic location as describe herein) will show increases in CpG methylation at or in proximity to the targeted genomic region as compared to non-targeting controls. Additionally, cells transfected with split effector entities, DNMT3L_fragment1 and DNMT3L_fragment2::dCas9, (with DNMT3L_fragment2::dCas9 targeted to a TUSC5 gene associated genomic location as described herein) will show reduction in off-target CpG methylation compared to cells transfected with DNMT3L(full length)::dCas9 and targeting gRNAs.

To determine differential CTCF binding within genomic locations targeted by gRNAs versus off-target binding by non-targeting (e.g. control) gRNAs, a CTCF chromatin immunoprecipitation-quantitative PCR assay (ChIP-qPCR) is performed. At 72 hours post-transfection, HEK293T cells are trypsinized and fixed with 1% formaldehyde in 10% fetal bovine serum and 90% phosphate buffered saline (PBS). Following glycine quenching of fixation, cells are pelleted by centrifugation, washed and then sonicated using a E220 evolution instrument (Covaris) to shear chromatin. Following another centrifugation step, the sheared chromatin supernatant is collected and added to pre-cleared magnetic beads (Thermo Fisher Scientific) complexed with a CTCF-specific antibody (Abcam). Following overnight incubation at 4° C., the CTCF-chromatin complexes bound to the beads are washed and resuspended in elution buffer. Subsequently, CTCF-chromatin complexes are eluted from the beads at 65° C. for 15 minutes. Crosslinks (from fixation) are then reversed, overnight at 65° C., and DNA is then purified by phenol:chloroform extraction. The resulting DNA serves as a template for SYBR Green (Thermo Scientific) qPCR using sequence-specific primers (IDT) flanking the CTCF-binding region. The primer sequences used for the amplification reaction are as follows: 5′-GCTGGAAACCTTGCACCTC-3′ and 5′-CGTTCAGGTTTGCGAAAGTA-3′.

Diminished input-normalized amplification, by 5% to 100%, indicates reduced CTCF binding due to targeted genetic modifications. Cells transfected with split effector entities, DNMT3L_fragment1 and DNMT3L_fragment2::dCas9 (with DNMT3L_fragment2::dCas9 targeted to the TUSC5 gene-associated genomic location) is considered to be likely to result in decrease in CTCF occupancy at or in proximity to the targeted genomic region(s) (i.e. CTCF anchor sites to which gRNAs are targeted) as compared to the non-targeting control(s).

To determine extent to which split effector entities (DNMT3L_fragment1::dCas9 and DNMT3L_fragment2::dCas9, targeted to the TUSC5-gene associated genomic location described herein), confer changes to proximity of CTCF-binding site upstream of TUSC5 to other CTCF binding sites, a 4C-seq assay is performed. At 72 hours post-transfection, 10⁶ cells are resuspended in 10% FBS/1×PBS. Formaldehyde is added to a concentration of 2% (wt/vol), and cells are incubated 10 minutes at 25° C. to crosslink. Formaldehyde is quenched by addition of glycine to a final concentration of 0.125 M. Cells are pelleted by centrifugation for 5 minutes at 500×g. Supernatants are discarded, and cell pellets are washed twice with 1×PBS followed by centrifugation for 5 minutes at 500×g. Cell pellets are resuspended in ice cold ice cold Hi-C lysis buffer (10 mM Tris-HCl pH 8, 10 mM NaCl, 0.2% IGEPAL CA-630, 1 Roche protease inhibitor tablet per 10 mL of buffer) and incubated for 30 minutes on ice. Nuclei are pelleted by centrifugation at 2500×g at 4° C. for 5 min. Pelleted nuclei are resuspended in 0.5% SDS and incubated for 7 minutes at 62° C. to disrupt nuclear membranes.

To quench SDS, Triton X100 is added to a final concentration of 0.1%, and mixtures are incubated at 37° C. for 15 minutes. Nuclei with semi-damaged nuclear membranes are then incubated with 200 U of NlaIII (NEB) for 4 h at 37° C. and then incubated with 200 U of NlaIII (NEB) for 15 hours at 37° C. Mixtures are incubated at 65° C. 20 minutes to heat inactivate the NlaIII. Nuclei are then pelleted by centrifugation at 2500×g for 5 minutes at 4° C.

Nuclei are incubated with 2000 U T4 DNA Ligase (NEB) for 6 hours at 25° C. while rotating to ligate DNA fragments that are in close proximity. Proteins are digested by incubating with Proteinase K (Promega) (at a final concentration of 20 mg/ml) at 55° C. for 30 minutes. Mixtures are incubated at 65° C. for 15 hours to reverse formaldehyde-dependent crosslinks. Mixtures are then treated with RNaseA (Sigma) followed by treatment with proteinase K (Life Technologies) according to manufacturer's recommendations.

DNA fragments are then purified by phenol-chloroform extraction (vol/vol) (Sigma) and precipitated in 0.3 M NaOAC pH 5.5 and ethanol (vol/vol) overnight at −20° C. DNA fragments are pelleted by centrifugation at 18000×g for 30 minutes at 4° C. Pellets are washed twice with 80% ethanol followed by centrifugation at 18000×g for 15 minutes at 4° C. Resulting pellets are resuspended in 10 mM Tris-HCl pH 7.5 and incubated with 50 U of BfaI (NEB) for 15 hours at 25° C. DNA fragments are then purified by phenol-chloroform extraction (vol/vol) (Sigma) and precipitated in 0.3 M NaOAC pH 5.5 and ethanol (vol/vol) overnight at −20° C. DNA fragments are pelleted by centrifugation at 18000×g for 30 minutes at 4° C. The pellets are washed twice with 80% ethanol followed by centrifugation at 18000×g for 15 minutes at 4° C. Resulting pellets are resuspended in 10 mM Tris-HCl pH 7.5.

Nuclei are incubated with 10,0000 U T4 DNA Ligase (NEB) for 15 hours at 16° C. to ligate intramolecular DNA fragments. DNA fragments are pelleted by centrifugation at 18000×g for 30 minutes at 4° C. Pellets are washed twice with 80% ethanol followed by centrifugation at 18000×g for 15 minutes 4° C. Resulting pellets are resuspended in 10 mM Tris-HCl pH 7.5. Primer sequences used for amplification reactions with a long template PCR reaction (Roche) are as follows: NB108898309_1f 5′-CCTAATTCAGGAGTGACATG-3′ and NB108898309_2r 5′-AGGGGAACTGTGAGGGAG-3′.

A diminished number of sequencing reads (e.g. between by about 5% to about 100%) indicates that CTCF binding site of interest is less frequently in proximity to (e.g. in a 4C-seq assay, proximity refers to two genomic loci that are located near one another based on protein interactions, and the relevant protein/DNA is/are crosslinked by formaldehyde) other CTCF binding sites. Cells transfected with the split effector entities, DNMT3L_fragment1 and DNMT3L_fragment2::dCas9 (with DNMT3L_fragment2::dCas9 targeted to a TUSC5 gene associated genomic location) are considered to be likely to show decreases in CTCF-mediated interactions between TUSC5 gene associated genomic location(s) as compared to the non-targeting control.

Among other things, the present disclosure, including as exemplified in present Example, provides systems that demonstrate direct methylation at targeted genomic CpGs via split effector entities fused to targeting moieties whose fragments reconstitute at the targeted location reasonably equivalent to that of the naturally occurring full length non-split protein, to the targeted genomic location. The present disclosure teaches that provided technologies, by assembling or reconstituting effector activity only when effector moiety fragments are co-localized (e.g., at the genomic location), may restrict specific biological activity to the vicinity of the genomic site. This strategy is considered to be likely to reduce non-specific methylation at non-targeted genomic CpG sites, below levels observed for cells transfected with DNMT3L (full length)::dCas9 and targeting gRNAs.

### Example #12.2 Split Effector Moieties for Epigenetic Modifications (MYC)

This Example describes two fragments of rat APOBEC protein. A first fragment is able to bind single-stranded DNA and a second fragment is fused to a targeting moiety via covalently tethering (e.g., fused) to a mutated Cas9 protein (Cas9 protein with D10A mutations) that is also covalently tethered to uracil glycosylase inhibitor protein (UGI). These entities are referred to as APOBEC_fragment1 and APOBEC_fragment2::Cas9_D10A::UGI.

Rat APOBEC protein is a cytidine deaminase that converts cytosine (“C”) to the RNA base, uracil (“U”). Targeting of a protein fusion of rat APOBEC covalently linked to Cas9 D10A linked to uracil glycosylase inhibitor protein (UGI), APOBEC1-Cas9_D19A-UGI, to a specific genomic location has been shown to result in conversion of genomic cytosine (“C”) bases to thymidine (“T”).

The present Example demonstrates disruption of a MYC gene-associated genomic location by epigenetic modification. A CTCF binding site is located upstream of the MYC gene, allowing enhancers within this particular genomic location to influence the MYC promoter. Disruption of a CTCF binding sequence (at either end) of this MYC-gene associated genomic location considered to be likely to reduce interaction of enhancers (within the genomic location) with MYC promoter and/or reduce expression of MYC.

Targeting of APOBEC_fragment2::Cas9_D10A::UGI to aMYC gene-associated genomic location is expected to reconstitute biochemical activity (conversion of genomic cytosine (“C”) to the RNA base uracil (“U”)) at the targeted location by binding to APOBEC_fragment1; the reconstituted biochemical activity is comparable to the APOBEC::Cas9_D10A::UGI protein targeted by gRNAs. This targeting will result in methylation of conversion of genomic cytosine to the RNA base uracil at or in proximity the MYC gene associated genomic location and/or reduced CTCF occupancy at the targeted genomic region, and/or decreased expression of MYC.

Production of Split Effector Moieties and Associated Components

All plasmids and guide RNAs (gRNA) are chemically synthesized from commercially available vendors. All agents are reconstituted in sterile water. Three plasmids (“Plasmid 1”; “Plasmid 2”; and “Plasmid 3”) are synthesized and each contains a dCas9 expression cassette, where Cas9_D10A expression driven by CMV enhancer and chicken beta-actin promoter with SV40 nuclear localization sequence (NLS) on N-terminus and a C-terminal linker (cctgcttctggcggaacttcatctgatggtggcacgtcagacggagggtcaagcaacacaggcggtagctctgacggaggga gctcagaaggcgaacctgcgcatgca).

Plasmid 1

In plasmid 1, the sequence of full length rat APOBEC (UniProtKB-P38483) with C-terminal SV40NLS follows the 3′ end of the C-terminal linker.

Plasmid 2

In plasmid 2, the sequence of rat APOBEC_fragment1 for split construct 1, as listed in Table 3, with C-terminal SV40 NLS follows the 3′ end of the C-terminal linker. In addition, in plasmid 2, the sequence of rat APOBEC_fragment2 for split construct 1, as listed in Table 1, is driven by an IRES promoter and has both N and C-terminal SV40 NLSes.

Plasmid 3

In plasmid 3, the sequence of rat APOBEC_fragment1 for split construct 2, as listed in Table 3, with C-terminal SV40 NLS follows the 3′ end of the C-terminal linker. In addition, in plasmid 3, the sequence of rat APOBEC_fragment2 for split construct 2, as listed in Table 3, is driven by an IRES promoter and has both N and C-terminal SV40 NLSes.

TABLE 3
Design of APOBEC split constructs.

| | APOBEC Fragment 1 | APOBEC Fragment 2 |
|---|---|---|
| Split construct #1 | APOBEC amino acids | APOBEC amino acids |
| | 2-168 | 169-229 |
| Split construct #2 | APOBEC amino acids | APOBEC amino acids |
| | 2-142 | 143-229 |

TABLE 4
Sequences of gRNAs targeting
putative CTCF sites associated with a MYC gene
associated genomic location

| ID | Guide RNA Sequence (5′-3′) |
|---|---|
| SACR-00002 | CTATTCAACCGCATAAGAGA |
| SACR-00011 | CGCTGAGCTGCAAACTCAAC |
| SACR-00015 | GCCTGGATGTCAACGAGGGC |
| SACR-00016 | GCGGGTGCTGCCCAGAGAGG |
| SACR-00017 | GCAAAATCCAGCATAGCGAT |

Exemplary plasmids are listed in Table 3 and described herein. HEK293T cells are serially transfected (either with a first plasmid, then a second plasmid, or with a second plasmid and then a first plasmid) with a first plasmid encoding APOBEC_fragment1 and a second plasmid encoding APOBEC_fragment2::Cas9_D10A::UGI, alternatively and/or additionally a plasmid encoding APOBEC(full length)::Cas9_D10A::UGI and either a non-targeting gRNA (“Non-targeting,” where the guide RNA sequence has no homology to the human genome) or a gRNA, as listed in Table 4, targeted at or near the putative CTCF binding sites of the MYC-associated genomic location encompassing the MYC gene, and/or full length APOBEC and at least one fragment, each tagged with different epitopes to facilitate distinguishing occupancy during, e.g. a competitive binding experiment. HEK293T cells are transfected first with plasmid encoding Cas9_D10A fusions, and then transfected 8 hours later with either a chemically synthesized gRNA targeting the CTCF binding site or a non-targeting (e.g. control) gRNA.

At 72 hours post-transfection, cells are harvested for RNA extraction and cDNA synthesis using commercially available reagents and protocols (Qiagen; Thermo Fisher Scientific) and genomic DNA is extracted (Qiagen). The resulting cDNA is used for quantitative real-time PCR (Thermo Fisher Scientific). MYC-specific quantitative PCR probes/primers (Thermo Fisher Scientific) are multiplexed with internal control quantitative PCR probes/primers for PPIB (Assay ID Hs00168719_m1, Thermo Fisher Scientific) using FAM-MGB and VIC-MGB dyes, respectively, and gene expression is subsequently analyzed by a real time PCR kit (Applied Biosystems, Thermo Fisher Scientific).

Cells transfected with split effector entities, APOBEC_fragment1 and APOBEC_fragment2::Cas9_D10A::UGI, (with APOBEC_fragment2::Cas9_D10A::UGI) targeted to the MYC gene associated genomic location are considered to be likely to show decrease in MYC expression as compared to non-targeting control.

To analyze conversion of cytosine (“C”) to uracil (“U”), gDNA extracted at 72 hours post-transfection (Qiagen) is used as template to amplify the CTCF-binding DNA region and multiple non-targeting DNA regions by a PCR kit (Promega). Base editing “C-to-U” is determined by sequencing of resultant PCR products. By aligning sequences of resultant PCR products to the original reference sequence of the amplified DNA region, “C-to-U” base editing is identified where thymidine (“T”) is sequenced in place of cytosine (“C”). Any number of non-zero “C-to-T” sequencing calls on a chromatogram indicate genetic modification by at least one of the split effector entities (e.g. APOBEC_fragment1::Cas9_D10A::UGI and APOBEC_fragment2::Cas9_D10A::UGI), or by the full length effector entity, APOBEC(full length)::Cas9_D10A::UGI.

Degree of reconstituted split effector entity, APOBEC_fragment1 and APOBEC_fragment2::Cas9_D10A::UGI, directing “C-to-U” base editing is subsequently ascertained by comparing number and position of “C-to-T” sequencing base calls in the MYC-targeted samples to those of the non-targeting control, and to those in cells transfected with APOBEC(full length)::Cas9_D10A::UGI and targeting gRNAs, where an integer increase in “C-to-T” base calls indicates increase in split effector entity targeted “C-to-U” base editing.

Cells transfected with the split effector entities, APOBEC_fragment1 and APOBEC_fragment2::Cas9_D10A::UGI (with APOBEC_fragment2::Cas9_D10A::UGI targeted to a MYC gene associated genomic location) are considered to be likely to show increase in “C-to-U” base editing at or in proximity to the targeted genomic region compared to non-targeting controls. Additionally, cells transfected with the split effector entities, APOBEC_fragment1 and APOBEC_fragment2::Cas9_D10A::UGI (with APOBEC_fragment2::Cas9_D10A::UGI targeted to a MYC gene associated genomic location) are considered to be likely to show reduction in off-target “C-to-U” base editing compared to cells transfected with APOBEC(full length)::Cas9_D10A::UGI and targeting gRNAs.

To determine differential CTCF binding at binding sites targeted by gRNAs versus non-targeting (e.g. control) gRNAs, a CTCF chromatin immunoprecipitation-quantitative PCR assay (ChIP-qPCR) is performed as described in Example 12.1. The resulting DNA serves as a template for SYBR Green (Thermo Scientific) qPCR using sequence-specific primers (IDT) flanking target CTCF-binding region(s). The primer sequences used for the amplification reaction(s) are as follows: 5′-GCTGGAAACCTTGCACCTC-3′ and 5′-CGTTCAGGTTTGCGAAAGTA-3′. Diminished input-normalized amplification (e.g. by 5% to about 100%), indicates reduced CTCF binding. It is considered to be likely that such reduced CTCF binding is due to targeted genetic modifications.

Cells Transfected with the Split Effector Entities

APOBEC_fragment1::Cas9_D10A::UGI and APOBEC_fragment2::Cas9_D10A::UGI, targeted to the MYC gene associated genomic location are considered to be likely to show decrease in CTCF occupancy at or in proximity to the targeted genomic region compared to non-targeting control.

To determine the extent to which split effector entities confer changes to proximity of a CTCF binding site upstream of MYC to other CTCF binding sites, a 4C-seq assay is performed as described in a previous Example, except that in the present Example, CviQI is utilized as second restriction enzyme instead of BfaI. Primer sequences used for amplification reactions with a long template PCR reaction (Roche) are as follows: NC74178114_if 5′-AGAGAGGCAGTCTGGTCATG-3′ and NC74178114_2r 5′-CCAGTGTCTTGCTTTCAAAT-3′. PCR products are multiplexed and sequenced with a 100-bp single-end Illumina Hi-Seq flow cell. Number of sequencing reads correlates with frequency of CTCF binding site(s) upstream of the MYC gene, localized in proximity to other CTCF binding sites.

A diminished number of sequencing reads (e.g. by about 5% to about 100%), indicates that a CTCF binding site of interest is less frequently in proximity to other CTCF binding sites as compared to, e.g. CTCF occupancy the relevant corresponding binding site in, e.g. a wild type cell line and/or, e.g. cells transfected with non-targeting gRNAs.

Cells Transfected with the Split Effector Entities

APOBEC_fragment1::Cas9_D10A::UGI and APOBEC_fragment2::Cas9_D10A::UGI, targeted to a MYC gene-associated CTCF anchor sequence-mediated conjunction are considered to be likely to show decrease in interaction frequency of a particular genomic location for the genomic region used as bait in the 4C assay as described in the present Example as compared to non-targeting control.

To the present inventors' knowledge, the present Example provides the first demonstration of directing base editing (C to U) at targeted genomic CpGs via split effector entities fused to targeting moieties and whose fragments reconstitute at the targeted location, thus restricting specific biochemical activity, equivalent to that of full length non-split protein, to the targeted genomic location. This strategy is considered to be likely to reduce non-specific base editing (C to U) at non-targeted genomic sites, below a level observed for cells transfected with APOBEC(full length)::Cas9_D10A::UGI and targeting gRNAs.

What is claimed is:

1. A system comprising:
a first composition comprising:
a first component comprising a first DNA targeting moiety capable of binding to a first target DNA site, operably linked to a first incomplete effector moiety; and
a second composition comprising:
a second component comprising a second DNA targeting moiety capable of binding to a second target DNA site adjacent to the first target site, operably linked to a second incomplete effector moiety,
wherein the first and second component are capable of interacting to provide an effector activity at or near the target site.

2. The system of the previous claim, wherein the effector activity modulates the DNA at or near the target site.

3. The system of any one of the previous claims, wherein the effector activity is selected from the group consisting of DNA methyltransferase, histone methyltransferase, deaminase, acetyltransferase, histone deacetylase, ligase, nuclease, phosphatase, recombinase, transposase, and polynucleotide kinase activity.

4. The system of any one of the previous claims, wherein the first and second components bind a DNA sequence comprising the first and second target sites.

5. The system of the previous claim, wherein the DNA sequence comprises a transcriptional control sequence.

6. The system of one of the previous claims, wherein the DNA sequence is genomic DNA.

7. The system of any one of the previous claims, wherein the first and second component prevents, inhibits, and/or interferes with an activity of an endogenous effector protein at the target site.

8. The system of any one of the previous claims, wherein the incomplete effector moieties are derived from at least one effector selected from the group consisting DNA methyltransferases (e.g., DNMT3a, DNMT3b, DNMTL, DRM2), DNA demethylation (e.g., the TET family, DME), histone methyltransferase, deaminase, acetyltransferase, histone deacetylase (e.g., HDAC1, HDAC2, HDAC3), sirtuin 1, 2, 3, 4, 5, 6, or 7, lysine-specific histone demethylase 1 (LSD1), histone-lysine-N-methyltransferase (Setdb1), euchromatic histone-lysine N-methyltransferase 2 (G9a), histone-lysine N-methyltransferase (SUV39H1), enhancer of zeste homolog 2 (EZH2), viral lysine methyltransferase (vSET), histone methyltransferase (SET2), protein-lysine N-methyltransferase (SMYD2), ligases, nucleases (e.g., endonucleases, T7, Cpf1, Cas9, zinc finger nuclease), phosphatases (e.g., alkaline phosphatases), recombinases (e.g., Cre), transposases (Tn3, Tn5, Sleeping Beauty), polynucleotide kinases (e.g., T4), enzymes with a role in DNA repair (e.g., RecA, N-glycosylase, AP-lyase), enzymes with a role in DNA demethylation (e.g., the TET family enzymes catalyze oxidation of 5-methylcytosine to 5-hydroxymethylcytosine and higher oxidative derivatives), and fragments or variants thereof.

9. The system of any one of the previous claims, wherein the incomplete effector moieties are derived from at least one effector selected from the group consisting of Table 1 from Park et al, Genome Biology, 2016, 17:183.

10. The system of any one of the previous claims, wherein the incomplete effector moieties are described in Example 1, Example 4, Example 6, Example 8, or Example 10.

11. The system of any one of the previous claims, wherein the DNA targeting moieties are described in Example 2, Example 3, Example 5, Example 7, or Example 9.

12. The system of any one of the previous claims, wherein at least one of the DNA targeting moieties are RNA.

13. The system of any one of the previous claims, wherein at least one composition of the system further comprises a nanoparticle, liposome, or exosome.

14. The system of any one of the previous claims, wherein at least one composition of the system further comprises a membrane penetrating polypeptide.

15. The system of any one of the previous claims, wherein the first and second composition are operably linked.

16. The system of any one of the previous claims, wherein the first and second compositions are each formulated as a separate pharmaceutical composition.

17. The system of any one of the previous claims, wherein the first and second compositions are formulated in a single pharmaceutical composition.

18. A system comprising:
a) a first nucleic acid sequence encoding a first incomplete effector moiety;
b) a first DNA targeting moiety that interacts with the first incomplete effector moiety and binds to a first target DNA site;
c) a second nucleic acid sequence encoding a second incomplete effector moiety; and
d) a second DNA targeting moiety that interacts with the second incomplete effector moiety and binds to a second target DNA site adjacent to the first target site, wherein the first and second incomplete effector moieties interact to provide an effector activity at or near the target site.

19. The system of the previous claim further comprising one or more vectors comprising one or more of a) through d).

20. The system of the previous claim, wherein the vector is an expression vector.

21. The system of any one of the previous claims, wherein a) and b) are operably linked and c) and d) are operably linked.

22. The system of any one of the previous claims, wherein a) comprises a first functional group and the first incomplete effector moiety comprises a first complementary functional group; and b) comprises a second functional group and the second incomplete effector moiety comprises a second complementary functional group, wherein the first functional group interacts with the first complementary functional group and the second functional group interacts with the second complementary functional group.

23. The system of any one of the previous claims, wherein the system is formulated as a pharmaceutical composition.

24. A pharmaceutical composition comprising a cell modified to express the system of any one of the previous claims.

25. A method of modifying a target site, the method comprising:
binding a first component comprising a first incomplete effector moiety with a nucleic acid sequence adjacent to the target site; and
binding a second component comprising a second incomplete effector moiety with a different nucleic acid sequence also adjacent to the target site,
wherein binding both components to the nucleic acid sequences allows interaction between the first and second components to induce effector activity at the target site.

26. The method of the previous claim, wherein the effector activity is selected from the group consisting of DNA methyltransferase, histone methyltransferase, deaminase, acetyltransferase, histone deacetylase, ligase, nuclease, phosphatase, recombinase, transposase, and polynucleotide kinase activity.

27. The method of the previous claim, wherein the effector activity at the target site modulates gene expression.

28. The method of the previous claim, wherein binding both components modulates chromatin topology and/or chromatin structure.

29. The method of the previous claim, wherein binding both components prevents, inhibits, and/or interferes with an activity of an endogenous effector protein at the target site.

30. A method of treating a disease or condition comprising administering the system of any one of the previous claims to a subject in need thereof.

31. The method of any one of the previous claims, wherein the system comprises a methyltransferase to treat a disease characterized by an overexpressed/dominant negative gene such as: an oncogene driven cancer (e.g MYC addicted cancers, Bcr-Abl), severe congenital neutropenia, and Huntington's chorea.

32. The method of any one of the previous claims, wherein the system comprises a demethylase to treat a disease characterized by under-expression of a gene: an imprinted disease (e.g. Prader Willi, Angelman Syndrome), a haploinsufficient disease (e.g Dravet's syndrome, Familial hypertriglyceridemia), Fragile X, Rett Syndrome, and a tumor suppressor that is underactive (e.g., retinoblastoma).


SEQUENCE LISTING (52 sequences)

SEQ ID NO: 1 - DNA, 200 bp, Artificial Sequence
  Description of Artificial Sequence Synthetic polynucleotide
  misc_feature (1)..(200): This sequence may encompass 100-200 nucleotides
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 2 - protein, 5 aa, Artificial Sequence
  Description of Artificial Sequence Synthetic peptide
CPRSC

SEQ ID NO: 3 - DNA, 15 bp, Artificial Sequence
  Description of Artificial Sequence Synthetic oligonucleotide
taacgtttatgttaa

SEQ ID NO: 4 - DNA, 15 bp, Artificial Sequence
  Description of Artificial Sequence Synthetic oligonucleotide
gacctcactccagaa

SEQ ID NO: 5 - DNA, 15 bp, Artificial Sequence
  Description of Artificial Sequence Synthetic oligonucleotide
gacctccggggtggg

SEQ ID NO: 6 - DNA, 15 bp, Artificial Sequence
  Description of Artificial Sequence Synthetic oligonucleotide
cggggtcggggtggt

SEQ ID NO: 7 - DNA, 15 bp, Artificial Sequence
  Description of Artificial Sequence Synthetic oligonucleotide
ctcacgtggagacgt

SEQ ID NO: 8 - DNA, 15 bp, Artificial Sequence
  Description of Artificial Sequence Synthetic oligonucleotide
tcccgccccggctcc

SEQ ID NO: 9 - DNA, 15 bp, Artificial Sequence
  Description of Artificial Sequence Synthetic oligonucleotide
cccctccaacgaaga

SEQ ID NO: 10 - DNA, 15 bp, Artificial Sequence
  Description of Artificial Sequence Synthetic oligonucleotide
tggagacataacctt

SEQ ID NO: 11 - DNA, 15 bp, Artificial Sequence
  Description of Artificial Sequence Synthetic oligonucleotide
tttcttagttaaccc

SEQ ID NO: 12 - DNA, 15 bp, Artificial Sequence
  Description of Artificial Sequence Synthetic oligonucleotide
ccaacaaattccaac

SEQ ID NO: 13 - DNA, 21 bp, Artificial Sequence
  Description of Artificial Sequence Synthetic primer
atttgctgatgaccaggtcct

SEQ ID NO: 14 - DNA, 21 bp, Artificial Sequence
  Description of Artificial Sequence Synthetic primer
cataaaccaatctcatgaggc

SEQ ID NO: 15 - DNA, 24 bp, Artificial Sequence
  Description of Artificial Sequence Synthetic primer
gctcactccaacagattcaagcag

SEQ ID NO: 16 - DNA, 24 bp, Artificial Sequence
  Description of Artificial Sequence Synthetic primer
gttggtagatgaaagggtcgagag

SEQ ID NO: 17 - DNA, 21 bp, Artificial Sequence
  Description of Artificial Sequence Synthetic primer
cacttgaagggtggtgccaag

SEQ ID NO: 18 - DNA, 21 bp, Artificial Sequence
  Description of Artificial Sequence Synthetic primer
cctgttgtcgccaacgaagtc

SEQ ID NO: 19 - DNA, 30 bp, Artificial Sequence
  Description of Artificial Sequence Synthetic primer
aggttytyatyatatayygaaagaatggga

SEQ ID NO: 20 - DNA, 27 bp, Artificial Sequence
  Description of Artificial Sequence Synthetic primer
ttraaaccatccatrratrrcctartt

SEQ ID NO: 21 - DNA, 19 bp, Artificial Sequence
  Description of Artificial Sequence Synthetic primer
aaagagttatgggyygaag

SEQ ID NO: 22 - DNA, 26 bp, Artificial Sequence
  Description of Artificial Sequence Synthetic primer
crrraaccaaaatcattctctaaaca

SEQ ID NO: 23 - DNA, 26 bp, Artificial Sequence
  Description of Artificial Sequence Synthetic primer
tgtttagagaatgattttggttyyyg

SEQ ID NO: 24 - DNA, 35 bp, Artificial Sequence
  Description of Artificial Sequence Synthetic primer
ctaccaacctaarattatttactatttcattccaa

SEQ ID NO: 25 - protein, 12 aa, Artificial Sequence
  Description of Artificial Sequence Synthetic peptide
AGSSGNGSSGNG

SEQ ID NO: 26 - DNA, 108 bp, Artificial Sequence
  Description of Artificial Sequence Synthetic polynucleotide
cctgcttctggcggaacttcatctgatggtggcacgtcagacggagggtcaagcaacacaggcggtagctctgacggagggagctcagaaggcgaacctgcgcatgca

SEQ ID NO: 27 - DNA, 20 bp, Artificial Sequence
  Description of Artificial Sequence Synthetic oligonucleotide
cagcggatttgggctcccgg

SEQ ID NO: 28 - DNA, 20 bp, Artificial Sequence
  Description of Artificial Sequence Synthetic oligonucleotide
cctcatcactacctgccacg

SEQ ID NO: 29 - DNA, 20 bp, Artificial Sequence
  Description of Artificial Sequence Synthetic oligonucleotide
catcactacctgccacgagg

SEQ ID NO: 30 - DNA, 20 bp, Artificial Sequence
  Description of Artificial Sequence Synthetic oligonucleotide
tgagactccagcatcccaca

SEQ ID NO: 31 - DNA, 20 bp, Artificial Sequence
  Description of Artificial Sequence Synthetic oligonucleotide
ccagagtagtccctggcacg

SEQ ID NO: 32 - DNA, 19 bp, Artificial Sequence
  Description of Artificial Sequence Synthetic primer
gctggaaaccttgcacctc

SEQ ID NO: 33 - DNA, 20 bp, Artificial Sequence
  Description of Artificial Sequence Synthetic primer
cgttcaggtttgcgaaagta

SEQ ID NO: 34 - DNA, 20 bp, Artificial Sequence
  Description of Artificial Sequence Synthetic primer
cctaattcaggagtgacatg

SEQ ID NO: 35 - DNA, 18 bp, Artificial Sequence
  Description of Artificial Sequence Synthetic primer
aggggaactgtgagggag

SEQ ID NO: 36 - DNA, 20 bp, Artificial Sequence
  Description of Artificial Sequence Synthetic oligonucleotide
ctattcaaccgcataagaga

SEQ ID NO: 37 - DNA, 20 bp, Artificial Sequence
  Description of Artificial Sequence Synthetic oligonucleotide
cgctgagctgcaaactcaac

SEQ ID NO: 38 - DNA, 20 bp, Artificial Sequence
  Description of Artificial Sequence Synthetic oligonucleotide
gcctggatgtcaacgagggc

SEQ ID NO: 39 - DNA, 20 bp, Artificial Sequence
  Description of Artificial Sequence Synthetic oligonucleotide
gcgggtgctgcccagagagg

SEQ ID NO: 40 - DNA, 20 bp, Artificial Sequence
  Description of Artificial Sequence Synthetic oligonucleotide
gcaaaatccagcatagcgat

SEQ ID NO: 41 - DNA, 20 bp, Artificial Sequence
  Description of Artificial Sequence Synthetic primer
agagaggcagtctggtcatg

SEQ ID NO: 42 - DNA, 20 bp, Artificial Sequence
  Description of Artificial Sequence Synthetic primer
ccagtgtcttgctttcaaat

SEQ ID NO: 43 - DNA, 1875 bp, Homo sapiens
tgaccgtgagcctgggtgaaagtgagttccccgttggaggcaacagacgaggagaggatggaaggcctggcccccaagaatgagccctgaggttcagggagcggctggagtgagccggccccagatctccgtccagctgcgggtcccagaggcctgggttacactcgcagctcctgggggaggcccttgacgtgcctcagttcccaaacaggaaccctgggaaggaccagagaagtgcctattgcgcagtgagtgcccgacacagctgcatgtggccggtatcacagggccctgggtaaactgaggcaggcgacacagctgcatgtggccggtatcacagggccctgggtaaactgaggcaggcgacacagctgcatgtggccggtatcacagggccctgggtaaactgaggcaggcgacacagctgcatgtggccgtatcacagggccctgggtaaactgaggcaggtgacacagctgcatgtggccggtatcacggggccctggataaacagaggcaggcgacacagctgcatgtggccggtatcacggggccctgggtaaactgaggcaggcgaggccacccccatcaagtccctcaggtctaggtttggcaggtttggcaaaaacacagcaacgctcggttaaatctgaatttcgggtaagtatatcctgggcctcatttggaagagacttagattaaaaaaaaaacgtcgagaccagcccggccaacacggtgaaaccccgtctctactaaaaatacaaaaaattagccaggcgcagtggctcacgcctgtgatcccagcactctgggaggctgaggcaggcggatcacccgaggtcagatgttcaagaccagcctggccgacagggcgaaacactgtctctactacaaatacaaaaattagccgggagtggtggcaggtgcctgtaatctcagctattcaggaggctgaggcaggagaatcacttgaacctgggaggcggaggttgccgtgagccgggatcacgccaccgcactccagcctgggcgatagagcaagactctgtctccaaaaaaataaattaaaaaacccacattgattatctgacatttgaatgcgattgtgcatcctgaattttgtctggaggccccacccgagccaatccagcgtcttgtcccccttctcccccttttcatcaacgccctgtgccaggggagaggaagtggagggcgctggccggccgtggggcaatgcaacggcctcccagcacagggctataagaggagccgggcgggcacggaggggcagagaccccggagccccagccccaccatgaccctcggccgccgactcgcgtgtcttttcctcgcctgtgtcctgccggccttgctgctggggggtgagtttttgagtccaacctcccgctgctccctctgtcccgggttctgttcccacctctccatagagggccccaccagtgtgggtccctcatcctcacaggggaggtgccagctgggacaaggagaccagaagagactgaggttctgagcggtgaagccaccaccaggagcccagagttggggtttgaaaaccggggagggggggggggtcgcaggtcgccctctgggttcaagtccaggtctgtctgtgccttggaggggcaccgtggggaggtccctttgcctctccgtgcctcagtttcctcatctgaacaacaggggtgcgaacggccccgatcccgtgggttcccggtgggggatcccgtgggttcccggtgggggatcccgtgggttcctggtgggggatccagaggccccgtggccgggaggggacaggctccttggcaggcactcagcacccgcacccggtgtgtccccaggcaccgcgctggc

SEQ ID NO: 44 - protein, 312 aa, Homo sapiens
WPSRLQMFFANNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACV

SEQ ID NO: 45 - protein, 386 aa, Spiroplasma monobiae
MSKVENKTKKLRVFEAFAGIGAQRKALEKVRKDEYEIVGLAEWYVPAIVMYQAIHNNFHTKLEYKSVSREEMIDYLENKTLSWNSKNPVSNGYWKRKKDDELKIIYNAIKLSEKEGNIFDIRDLYKRTLKNIDLLTYSFPCQDLSQQGIQKGMKRGSGTRSGLLWEIERALDSTEKNDLPKYLLMENVGALLHKKNEEELNQWKQKLESLGYQNSIEVLNAADFGSSQARRRVFMISTLNEFVELPKGDKKPKSIKKVLNKIVSEKDILNNLLKYNLTEFKKTKSNINKASLIGYSKFNSEGYVYDPEFTGPTLTASGANSRIKIKDGSNIRKMNSDETFLYIGFDSQDGKRVNEIEFLTENQKIFVCGNSISVEVLEAIIDKIGG

SEQ ID NO: 46 - protein, 327 aa, Haemophilus parahaemolyticus
MIEIKDKQLTGLRFIDLFAGLGGFRLALESCGAECVYSNEWDKYAQEVYEMNFGEKPEGDITQVNEKTIPDHDILCAGFPCQAFSISGKQKGFEDSRGTLFFDIARIVREKKPKVVFMENVKNFASHDNGNTLEVVKNTMNELDYSFHAKVLNALDYGIPQKRERIYMICFRNDLNIQNFQFPKPFELNTFVKDLLLPDSEVEHLVIDRKDLVMTNQEIEQTTPKTVRLGIVGKGGQGERIYSTRGIAITLSAYGGGIFAKTGGYLVNGKTRKLHPRECARVMGYPDSYKVHPSTSQAYKQFGNSVVINVLQYIAYNIGSSLNFKPY

SEQ ID NO: 47 - DNA, 1875 bp, Homo sapiens
cctattcttcataccccttatcacagctgcaactactcatttacttgtctgacaatttgatttatgtccacctactttgctaggtactaagttcaatgctggcagtcgtttcttctttttttttcttttctgttttgctcaccgatttctcgttagcacttagcacagtgtctggcacacgatagatgctccgtcaacttctcagttggataccagcatcccgaagggaacatggattaaggcagctataagcacggtgtaaaaacaggaataagaaaaagttgaggtttgtttcacagtggaatgtaaagggttgcaaggaggtgcatcggcccctgtggacaggacgcatgactgctacacacgtgttcaccccaccctctggcacagggtgcacatacagtaggggcagaaatgaacctcaagtgcttaacacaatttttaaaaaatatatagtcaagtgaaagtatgaaaatgagttgaggaaaggcgagtacgtgggtcaaagctgggtctgaggaaaggctcacattttgagatcccgactcaatccatgtcccttaaagggcacagggtgtctccacagggccgcccaaaatctggtgagagagggcgtagacgcctcaccttctgcctctacgggtcacaaaagcctgggtcaccctggttgccactgttcctagttcaaagtcttcttctgtctaatccttcacccctattctcgccttccactccacctcccgctcagtcagactgcgctactttgaaccggaccaaaccaaaccaaaccaaaccaaaccaaaccagaccagacaccccctcccgcggaatcccagagaggccgaactgggataaccggatgcatttgatttcccacgccactgagtgcacctctgcagaaatgggcgttctggccctcgcgaggcagtgcgacctgtcaccgcccttcagccttcccgccctccaccaagcccgcgcacgcccggcccgcgcgtctgtctttcgacccggcaccccggccggttcccagcagcgcgcatgcgcgcgctcccaggccacttgaagagagagggcggggccgaggggctgagcccgcggggggagggaacagcgttgatcacgtgacgtggtttcagtgtttacacccgcagcgggccgggggttcggcctcagtcaggcgctcagctccgtttcggtttcacttccggtggagggccgcctctgagcgggcggcgggccgacggcgagcgcgggcggcggcggtgacggaggcgccgctgccagggggcgtgcggcagcgcggcggcggcggcggcggcggcggcggcggaggcggcggcggcggcggcggcggcggcggctgggcctcgagcgcccgcagcccacctctcgggggcgggctcccggcgctagcagggctgaagagaagatggaggagctggtggtggaagtgcggggctccaatggcgctttctacaaggtacttggctctagggcaggccccatcttcgcccttccttccctcccttttcttcttggtgtcggcgggaggcaggcccggggccctcttcccgagcaccgcgcctgggtgccagggcacgctcggcgggatgttgttgggagggaaggactggacttggggcctgttggaagcccctctccgactccgagaggccctagcgcctatcgaaatgagagaccagcgaggagagggttctctttcggcgccgagccccgccggggtgagctggggatgggcgagggccggcggcaggtactagagccgggcgggaagggccgaaatcggcgctaagtgacggcgatggcttattccccctttcctaaacatcatctcccagcgggatccgggcctgt

SEQ ID NO: 48 - DNA, 1875 bp, Homo sapiens
tctcaacattttgtaccgtggcccacaggactgggtggaagacaggacagctcagagcagggagtgaaacaaaatctttgatggttctctagggaagcctggaaatctgagacagggcaaagagcagggagtctgatgagcacaaaattaaaatacccaaaccagcctgccgggtcaggggaaaaggaagcaggattgggcacatgaagaacagatgacaaaactgaggctcagagaggctgagagagacactgctgaggtcacacagccaggagtcggcaggaagccctgaacccaagcagggctggcctggctactagcctgggctcagtcctttagattggacttcaaagtatctgctccagatgacgctggcattgggaggagggcatgataatccctaacatccacacagggacaacagcacttggaggcagatgtgcttctcacttcaaggaaactgaggcagagagggagaggtggtcaggactcaagcccagtaattcatttgccaaatcctgtttccttttcccccaacccagcgggctggtttgggtattttcagtttgtgcccatcagagtccctgcagagtgctctccactgtgtctcagatctctgtgtccccagagaaccatcaaaggttttgtttcactccctgctctgggctgtcttccacccagccctgtgaacctcagtgcaggttggccgcccccttagagggaggctaatcagggaagttctgagtcaggtcgtcctggaaaaactacattgacctctggagctcaagagaggccaggggacaaacacatggctcagcacacaggaaggcaggtgtgggtattgagtgacctgagggagaaatccctatttatcatccatcatgtggtcctgctgtgacaaacccgtgtggcagccacccacagcagatccacaacgtgagaggcccgctgaaacaagggtgctggcattggttactcagcctggccctccctgccttgcggggtgacctgcctggtcctggcctctctgtgagttgggccctgctgggtggtccctagtgtgcccagccctgaggctggtgagaggccaggctgtgaggtgtgaggaacttacctgcgtctccatggaaggtgccctccgcatcgttgggccagatctgcctggtcttgcagatgcccacgatggtggcctctgacacgacgactgggcagtgccggtgacgcttatggcactgcggcgtggtaggggcagaggggggaggttgcttctgtcggaggactgctgcgagttctgccagagagagcagctcttgtcccggaacatgaggtaggtggtggggcttggggacacgcggctggactggccggagaagtcctcctggccggaggggagccaagtgttcctgttccaggactgcagaactggcccagacctctgtattggaaaggtctttatggaccagggagtccggtgtcttttttacgggggacccctgggctgcgagttgcacagtccaattcgctgttgttagggcctcagtttcccaaaaggcacagggacggggggagggtggcggctcgatgggggagccgcctccagggggcccccccgccctgtgcccacggcgcggcccctttaagaggcccgcctggctccgtcatccgcgccgcggccacctccccccggccctccccttcctgcggcgcagagtgcgggccgggcgggagtgcggcgagagccggctggctgagcttagcgtccgaggaggcggcggcggcggcggcggcacggcggcggcggggctgtggggcggtgcggaagcgagaggcgaggagcgcgcgggccgtggccagagtctggcggcggcctggcgg

SEQ ID NO: 49 - protein, 608 aa, Nicotiana tabacum
MDNNLSGEDNDNIDWDTEDELEIQEIQDTTFSSCMDLRTTGQHTVRCDGEASSSSVPCQSKFIQQFVVMGFPEASIAKAIEQNGEDSDLVLDALLTFKALEDSPEEQPSASPQPCINSDDSSSEYNENLLDDVYEEDSWSSDSDSCRNSAKQCYVKVESSSSSEKEQTLLFLASMGYPAEEASIAMERCGPEASVAELTDFICAAQMARAEDVYLPEDVKPKLNHILNGSGGYKKRKMYNELCKRKKAKAIFEEETIRLPKPMIGFGVPTEPLPAMVRRTLPEQAVGPPFFYYENVALAPKGVWDTISRFLYDIEPEFVDSKYFCAAARKRGYIHNLPVENRFPLFPLAPRTIHEALPLSKKWWPSWDPRTKLNCLQTAIGSAQLTNRIRKAVEDFDGEPPMRVQKFVLDQCRKWNLVWVGRNKVAPLEPDEVEMLLGFPKNHTRGGGISRTDRYKSLGNSFQVDTVAYHLSVLKDLFPGGINVLSLFSGIGGGEVALYRLGIPLNTVVSVEKSEVNRDIVRSWWEQTNQRGNLIHFNDVQQLNGDRLEQLIESFGGFDLVIGGSPCNNLAGSNRVSRDGLEGKESSLFYDYVRILDLVKSIMSRHKH

SEQ ID NO: 50 - protein, 626 aa, Arabidopsis thaliana
MVIWNNDDDDFLEIDNFQSSPRSSPIHAMQCRVENLAGVAVTTSSLSSPTETTDLVQMGFSDEVFATLFDMGFPVEMISRAIKETGPNVETSVIIDTISKYSSDCEAGSSKSKAIDHFLAMGFDEEKVVKAIQEHGEDNMEAIANALLSCPEAKKLPAAVEEEDGIDWSSSDDDTNYTDMLNSDDEKDPNSNENGSKIRSLVKMGFSELEASLAVERCGENVDIAELTDFLCAAQMAREFSEFYTEHEEQKPRHNIKKRRFESKGEPRSSVDDEPIRLPNPMIGFGVPNEPGLITHRSLPELARGPPFFYYENVALTPKGVWETISRHLFEIPPEFVDSKYFCVAARKRGYIHNLPINNRFQIQPPPKYTIHDAFPLSKRWWPEWDKRTKLNCILTCTGSAQLTNRIRVALEPYNEEPEPPKHVQRYVIDQCKKWNLVWVGKNKAAPLEPDEMESILGFPKNHTRGGGMSRTERFKSLGNSFQVDTVAYHLSVLKPIFPHGINVLSLFTGIGGGEVALHRLQIKMKLVVSVEISKVNRNILKDFWEQTNQTGELIEFSDIQHLTNDTIEGLMEKYGGFDLVIGGSPCNNLAGGNRVSRVGLEGDQSSLFFEYCRILEVVRARMRGS

SEQ ID NO: 51 - DNA, 1560 bp, Arabidopsis thaliana
gttatctaaataaaactaggccatccatggatggtttcaattttttttttcatatgaaagaaaagttaaatttcatttcacaataaccattgattactaaatttagtaaagaatcaattgggtttagtgtttacttgtttaaggttttttttttttcttttgttatggttctatactaatatcaaagagttatgggccgaagcccatacatctttccgtcgagaatctcatatattctttatcgaagcccatacatctttccgtcgagaatctcatatataccttatcccattcaacattcatacgagcgccgctctagggtttttgcttttcgccattggtccaagtgctatttggttgtttaaggttgcttttagcacacaactttaatattatttttatgtttttcttcttacgatttatcgatttgtgggatactgacaatcagattattgttgttttttccagccaaatatcagatcttgcgccgctctttatcccattcaacattcatacgagcaccgctttacggtttttgcttttcgacattggtcgaagtgctatttggttgtttaaggttgcttttagcacacaactttaatattatttttatgttttcttcttacgatttatcgatttgtaggatactgacaatcagatttttgttgtttttttcagccaaaaatcagatttttttacattttgtttagagaatgattttggttcccgatttgtctgtttttcgcttatgtgtaaagtactttgaaaaatattgtgttaactctacaatgggtatcccaaatttttgagttcttttgtcttgttcgttgtcgagacactagaaatgttaatttaattctcttcttccaaaaagaaccatttactggttcattctctacttgaattttattctggttgtatttcttttccagtataaagcagattgttttttgttatttttcagtttagattggctttgtctcttttgagttgttgcaattgtcaaactttggaatgaaatagtaaataatcttaggttggtagtaaatcttaacattgtgtttttggggcataatttatcgataaaatcttcagcattaaaaccaaaaagaaaaaactttttaagtcttttttgtttggtggttaatataaagtttatacgtgtattaatttgatcacactcactatatgtccagggagctaaacctctatatcgagtactaatagtatgcaatgtccaggttattgcattgagggaaaatgaatggacaaggtgatttggatgcggttggaaacattccaaaaccaggtgaagctgaaggcgatgagattgatatgattaatgatatgtctggtgttaatgatcaagatggtggaaggatgagaagaacccataggcgcactgcttatcaaactcaagaacttgaaaagtttgttcactttcttcttcatttcatcatcatgcaacatttcctattattttttttatttttttattttgagtttggaatgtttctctttactttgctctttactttaaaatgagtgtagtttctacat

SEQ ID NO: 52 - protein, 1987 aa, Arabidopsis thaliana
MNSRADPGDRYFRVPLENQTQQEFMGSWIPFTPKKPRSSLMVDERVINQDLNGFPGGEFVDRGFCNTGVDHNGVFDHGAHQGVTNLSMMINSLAGSHAQAWSNSERDLLGRSEVTSPLAPVIRNTTGNVEPVNGNFTSDVGMVNGPFTQSGTSQAGYNEFELDDLLNPDQMPFSFTSLLSGGDSLFKVRQYGPPACNKPLYNLNSPIRREAVGSVCESSFQYVPSTPSLFRTGEKTGFLEQIVTTTGHEIPEPKSDKSMQSIMDSSAVNATEATEQNDGSRQDVLEFDLNKTPQQKPSKRKRKFMPKVVVEGKPKRKPRKPAELPKVVVEGKPKRKPRKAATQEKVKSKETGSAKKKNLKESATKKPANVGDMSNKSPEVTLKSCRKALNFDLENPGDARQGDSESEIVQNSSGANSFSEIRDAIGGTNGSFLDSVSQIDKTNGLGAMNQPLEVSMGNQPDKLSTGAKLARDQQPDLLTRNQQCQFPVATQNTQFPMENQQAWLQMKNQLIGFPFGNQQPRMTIRNQQPCLAMGNQQPMYLIGTPRPALVSGNQQLGGPQGNKRPIFLNHQTCLPAGNQLYGSPTDMHQLVMSTGGQQHGLLIKNQQPGSLIRGQQPCVPLIDQQPATPKGFTHLNQMVATSMSSPGLRPHSQSQVPTTYLHVESVSRILNGTTGTCQRSRAPAYDSLQQDIHQGNKYILSHEISNGNGCKKALPQNSSLPTPIMAKLEEARGSKRQYHRAMGQTEKHDLNLAQQIAQSQDVERHNSSTCVEYLDAAKKTKIQKVVQENLHGMPPEVIEIEDDPTDGARKGKNTASISKGASKGNSSPVKKTAEKEKCIVPKTPAKKGRAGRKKSVPPPAHASEIQLWQPTPPKTPLSRSKPKGKGRKSIQDSGKARGPSGELLCQDSIAEIIYRMQNLYLGDKEREQEQNAMVLYKGDGALVPYESKKRKPRPKVDIDDETTRIWNLLMGKGDEKEGDEEKDKKKEKWWEEERRVFRGRADSFIARMHLVQGDRRFSPWKGSVVDSVIGVFLTQNVSDHLSSSAFMSLAARFPPKLSSSREDERNVRSVVVEDPEGCILNLNEIPSWQEKVQHPSDMEVSGVDSGSKEQLRDCSNSGIERFNFLEKSIQNLEEEVLSSQDSFDPAIFQSCGRVGSCSCSKSDAEFPTTRCETKTVSGTSQSVQTGSPNLSDEICLQGNERPHLYEGSGDVQKQETTNVAQKKPDLEKTMNWKDSVCFGQPRNDTNWQTTPSSSYEQCATRQPHVLDIEDFGMQGEGLGYSWMSISPRVDRVKNKNVPRRFFRQGGSVPREFTGQIIPSTPHELPGMGLSGSSSAVQEHQDDTQHNQQDEMNKASHLQKTFLDLLNSSEECLTRQSSTKQNITDGCLPRDRTAEDVVDPLSNNSSLQNILVESNSSNKEQTAVEYKETNATILREMKGTLADGKKPTSQWDSLRKDVEGNEGRQERNKNNMDSIDYEAIRRASISEISEAIKERGMNNMLAVRIKDFLERIVKDHGGIDLEWLRESPPDKAKDYLLSIRGLGLKSVECVRLLTLHNLAFPVDTNVGRIAVRMGWVPLQPLPESLQLHLLELYPVLESIQKFLWPRLCKLDQRTLYELHYQLITFGKVFCTKSRPNCNACPMRGECRHFASAYASARLALPAPEERSLTSATIPVPPESYPPVAIPMIELPLPLEKSLASGAPSNRENCEPIIEEPASPGQECTEITESDIEDAYYNEDPDEIPTIKLNIEQFGMTLREHMERNMELQEGDMSKALVALHPTTTSIPTPKLKNISRLRTEHQVYELPDSHRLLDGMDKREPDDPSPYLLAIWTPGETANSAQPPEQKCGGKASGKMCFDETCSECNSLREANSQTVRGTLLIPCRTAMRGSFPLNGTYFQVNELFADHESSLKPIDVPRDWIWDLPRRTVYFGTSVTSIFRGLSTEQIQFCFWKGFVCVRGFEQKTRAPRPLMARLHFPASKLKNNKT